(19) United States
(12) Patent Application Publication
(10) Pub. No.: US 2026/0193710 A1
(43) Pub. Date: Jul. 9, 2026

(54) METHODS AND MODELS FOR POST-OPERATIVE RECURRENCE IN CROHN'S DISEASE

(71) Applicant: CEDARS-SINAI MEDICAL CENTER, Los Angeles, CA (US)

(72) Inventors: Phillip GU, Los Angeles, CA (US); Dermot P. MCGOVERN, Los Angeles, CA (US); Shishir DUBE, Los Angeles, CA (US)

(21) Appl. No.: 19/132,747

(22) PCT Filed: Nov. 28, 2023
(86) PCT No.: PCT/US2023/081388

§ 371 Date: May 23, 2025

Related U.S. Application Data
(60) Provisional application No. 63/385,380, filed on Nov. 29, 2022.
(60) Provisional application No. 63/500,494, filed on May 5, 2023.
(60) Provisional application No. 63/510,591, filed on Jun. 27, 2023.

(51) Int. Cl. C12Q 1/6883; G01N 33/68; G16B 20/20; G16H 50/20

(52) U.S. Cl. CPC C12Q 1/6883; G01N 33/6893; G16B 20/20; G16H 50/20; C12Q 2600/118; C12Q 2600/156; G01N 2800/065; G01N 2800/52

(57) ABSTRACT

Provided herein are methods, systems, compositions and kits for modeling post-operative recurrence (POR) or postoperative prophylaxis persistence (POPP) in Crohn's disease patients. The present disclosure provides clinical, serologic, and genetic factors predictive of POR in patients with Crohn's disease. Also provided are clinical, serologic, and genetic factors predictive of POPP in Crohn's disease patients. The clinical, serologic, and genetic factors of the present disclosure are useful for selecting for prognosing, diagnosing, treatment, treating, monitoring a treatment, or optimizing a treatment for a Crohn's disease patient.

### CROSS-REFERENCE TO RELATED APPLICATIONS

This application claims benefit of U.S. Provisional Patent Application No. 63/385,380, filed on Nov. 29, 2022, U.S. Provisional Patent Application No. 63/500,494, filed on May 5, 2023, and U.S. Provisional Patent Application No. 63/510,591, filed Jun. 27, 2023, each of which is incorporated herein by reference in its entirety.

### STATEMENT AS TO FEDERALLY SPONSORED RESEARCH

This invention was made with government support under Grant No. U01DK062413, and P01DK046763, awarded by National Institutes of Health. The government has certain rights in the invention.

### SUMMARY

In certain aspects, disclosed herein is a method of treating Crohn's disease (CD) in a subject, the method comprising: administering to the subject a therapeutically effective amount of a therapeutic agent for CD, wherein one or more post-operative prophylaxis persistence (“POPP”) markers were detected in the subject. In some embodiments, the method comprises determining the therapeutic agent to have prophylactic persistence in treating the CD in the subject based, at least in part, on the one or more POPP markers detected in the subject.

In certain aspects, disclosed herein is a method of determining prophylactic persistence of therapeutic agent in treating Crohn's disease (CD) in a subject that has undergone operation, the method comprising: detecting one or more post-operative prophylaxis persistence (“POPP”) markers in the subject; and determining a likelihood that the therapeutic agent exhibits the prophylactic persistence in treating the CD in the subject based, at least in part, on the one or more POPP markers detected in the subject, wherein the likelihood is compared with a control subject with the CD and that has received a surgical resection to treat the CD. In some embodiments, the method comprises detecting the one or more POPP markers from a sample obtained from the subject. In some embodiments, the sample comprises blood, serum, plasma, sweat, hair, tears, urine, saliva, stool, or combination thereof. In some embodiments, the one or more POPP markers comprises one or more POPP clinical factors, one or more POPP serological factors, one or more POPP genetic factors, or any combination thereof. In some embodiments, the one or more POPP clinical factors comprises an active smoker status, disease location, a failure of one or more therapeutic agents to treat the CD, resection length of operation, or lymph node granuloma, or any combination thereof. In some embodiments, the method comprises obtaining the one or more POPP clinical factors from the subject directly or reviewing medical records of the subject to obtain the one or more POPP clinical factors. In some embodiments, the disease location is an ileocolonic region of an intestine of the subject. In some embodiments, the likelihood of the POPP of the therapeutic agent is high based, at least in part, on the one or more POPP clinical factors detected in the subject comprising the disease location in the ileocolonic region of the intestine of the subject. In some embodiments, the likelihood of the POPP of the therapeutic agent is high based, at least in part, on the one or more POPP clinical factors detected in the subject comprising the active smoker status. In some embodiments, an active smoker status is defined by smoking at least one cigarette per week. In some embodiments, the therapeutic agent has a low likelihood of the POPP based, at least in part, on the one or more POPP clinical factors detected in the subject comprising the lymph node granuloma. In some embodiments, the failure of the one or more therapeutic agents to treat the CD is a recurrence or flare up of the CD following successful remission of the CD following administration of the one or more therapeutic agents to the subject. In some embodiments, the one or more therapeutic agents comprises a steroid, an immunomodulator, 5-aminosalicylate, or combinations thereof. In some embodiments, the one or more therapeutic agents comprises one or more biologic therapeutic agents. In some embodiments, the one or more biologic therapeutic agents comprises two or more biologic therapeutic agents. In some embodiments, the one or more biologic therapeutic agents comprises three or more biologic therapeutic agents. In some embodiments, the one or more POPP serological factors comprises ANCA seronegativity, ASCA IgA seronegativity, or ASCA IgG seronegativity, or any combination thereof. In some embodiments, the likelihood of the POPP of the therapeutic agent is high based, at least in part, on the one or more POPP serological factors detected in the subject comprising the ASCA seropositivity. In some embodiments, the likelihood of the POPP of the therapeutic agent is high based, at least in part, on the one or more POPP serological factors detected in the subject comprising the ANCA seronegativity. In some embodiments, the likelihood of the POPP of the therapeutic agent is high based, at least in part, on the one or more POPP serological factors detected in the subject comprising the ASCA seropositivity and the ANCA seronegativity. In some embodiments, the one or more POPP serological factors is detected in the subject by a method comprising introducing a sample obtained from the subject to a detection agent configured to bind ANCA, ASCA IgA, ASCA IgG under conditions sufficient to detect the ANCA, ASCA IgA, ASCA IgG, or any combinations thereof. In some embodiments, the method comprises detecting the one or more POPP serological factors comprising performing an enzyme-linked immunosorbent assay (ELISA). In some embodiments, the measuring the seropositivity or the seronegativity comprises calculating a percentage of a reference value from pooled sera obtained from a patient population diagnosed with CD. In some embodiments, the one or more POPP genetic factors comprises an absence of absence of an adenosine (“A”) at a polymorphism position comprising rs6811007, or a proxy polymorphism position in linkage disequilibrium therewith as determined with an r² of at least 0.85, or a combination thereof. In some embodiments, the one or more POPP genetic factors is detected in the subject by a method comprising detecting a nucleic acid sequence. In some embodiments, the detecting the nucleic acid sequence comprises performing quantitative polymerase chain reaction (qPCR), nucleic acid sequencing, or hybridization to immobilized nucleic acid sequences on a nucleic acid array. In some embodiments, the one or more POPP histological factors comprises operative visceral adipose tissue (VAT) quality, wherein the operative VAT is defined by a reduced radiodensity. In some embodiments, the reduced radiodensity comprises a VAT radiodensity of less than or equal to about 0.631 Hounsfield unit (HU). In some embodiments, the reduced radiodensity comprises a ratio of VAT to SAT radiodensity of up to 5.559. In some embodiments, the reduced radiodensity comprises a VAT radiodensity of about 0.43 to about 0.7 HU. In some embodiments, the reduced radiodensity comprises a ratio of VAT to SAT radiodensity of ranging from 1.18-6. In some embodiments, the likelihood of the POPP of the therapeutic agent is high based, at least in part, on the one or more POPP histological factors detected in the subject comprising the operative VAT quality. A method of treating Crohn's disease (CD) in a subject, the method comprising: administering to the subject a therapeutically effective amount of a therapeutic agent for treatment of the CD, wherein one or more post-operative recurrence (“POR”) markers were detected in the subject. In some embodiments, the method comprises determining the subject to have a likelihood of the POR based, at least in part, on the one or more POR markers detected in the subject relative to a control subject having the CD that has received a resection to treat the CD. In some embodiments, the one or more POPP genetic factors comprises a single nucleotide polymorphism at a polymorphism position comprising rs6811007 or a proxy polymorphism position in linkage disequilibrium therewith as determined with an r² of at least 0.85, or a combination thereof. In some embodiments, the one or more POPP genetic factors comprises a single nucleotide polymorphism at a polymorphism position comprising rs1533236, or a proxy polymorphism position in linkage disequilibrium therewith as determined with an r² of at least 0.85, or a combination thereof. In some embodiments, the one or more POPP genetic factors comprises a single nucleotide polymorphism at a polymorphism position comprising rs6811007 or a proxy polymorphism position in linkage disequilibrium therewith as determined with an r² of at least 0.85, or a combination thereof; a single nucleotide polymorphism at a polymorphism position comprising rs1533236, or a proxy polymorphism position in linkage disequilibrium therewith as determined with an r² of at least 0.85, or a combination thereof; or both. In some embodiments, the polymorphism position comprising rs1533236 comprises an absence of an adenosine (“A”).

In certain aspects, disclosed herein is method of determining the prognosis of post-operative recurrence (“POR”) of Crohn's disease (CD) in a subject, the method comprising: detecting one or more POR markers in the subject; and determining the subject to have a likelihood of POR based, at least in part on the one or more POR markers detected in the subject, relative to a control subject having the CD that has received a resection to treat the CD. In some embodiments, the method comprises detecting the one or more POR markers from a sample obtained from the subject. In some embodiments, the sample comprises blood, serum, plasma, sweat, hair, tears, urine, saliva, stool, or any combination thereof. In some embodiments, the one or more POR markers comprises one or more POR clinical factors, one or more POR serological factors, one or more POR genetic factors, or one or more POR histological factors, or any combination thereof. In some embodiments, the one or more POR clinical factors comprises active smoking, disease location, a failure of a therapeutic agent for treatment of the CD, resection length of operation, or lymph node granuloma, or any combination thereof. In some embodiments, the disease location is an ileocolonic region of an intestine of the subject. In some embodiments, the method comprises identifying the likelihood of the subject of the POR is high relative to the control subject, wherein the one or more POR clinical factors detected in the subject comprises the disease location, wherein the disease location is an ileocolonic region of an intestine of the subject. In some embodiments, the method comprises identifying the likelihood of the subject of the POR is low relative to the control subject, wherein the one or more POR clinical factors detected in the subject comprises disease location in the ileal region. In some embodiments, the likelihood is high relative to the control subject based, at least in part, on the one or more POR clinical factors detected in the subject, wherein the one or more POR clinical factors comprises an active smoker. In some embodiments, an active smoker status is defined by smoking at least one cigarette per week. In some embodiments, the method comprises identifying the likelihood of the subject as of the POR is high relative to the control subject, wherein the one or more POR clinical factors detected in the subject comprises the lymph node granuloma. In some embodiments, the failure of the one or more therapeutic agents to treat the CD is a recurrence or flare up of the CD following successful remission of the CD following administration of the one or more therapeutic agents to the subject. In some embodiments, the one or more therapeutic agents comprises a steroid, an immunomodulator, 5-aminosalicylate, or combinations thereof. In some embodiments, the one or more therapeutic agents comprises one or more biologic therapeutic agents. In some embodiments, the one or more biologic therapeutic agents comprises two or more biologic therapeutic agents. In some embodiments, the one or more biologic therapeutic agents comprises three or more biologic therapeutic agents. In some embodiments, the method comprises identifying the likelihood of the subject of the POR is high relative to the control subject, wherein the one or more POR clinical factors detected in the subject comprises a failure of the one or more therapeutic agents to treat the CD, wherein the one or more therapeutic agents comprises one or more biologic therapeutic agents to treat the CD. In some embodiments, the one or more POR serological factors is detected in the subject by a method comprising introducing a sample obtained from the subject to a detection agent configured to bind ANCA, ASCA IgA, ASCA IgG under conditions sufficient to detect the ANCA, ASCA IgA, ASCA IgG, or any combinations thereof. In some embodiments, the method comprises identifying the subject as having a high likelihood of the POR wherein if the one or more POR clinical factors detected in the subject comprises ANCA seropositivity, ASCA IgA seropositivity, or ASCA IgG seropositivity, or any combination thereof. In some embodiments, the method comprises identifying the subject as having a high likelihood of the POR wherein if the one or more POR clinical factors detected in the subject comprises ASCA seropositivity. In some embodiments, the method comprises identifying the subject as having a high likelihood of the POR wherein if the one or more POR clinical factors detected in the subject comprises ANCA seronegativity. In some embodiments, the method comprises identifying the subject as having a high likelihood of the POR wherein if the one or more POR clinical factors detected in the subject comprises ASCA seropositivity and ANCA seronegativity. In some embodiments, the one or more POR genetic factors comprises an absence of presence of an adenosine (“A”) at a polymorphism position comprising rs6811007, or a proxy polymorphism position in linkage disequilibrium therewith as determined with an r² of at least 0.85, or a combination thereof. In some embodiments, the one or more POR genetic factors is detected in the subject by a method comprising detecting a nucleic acid sequence. In some embodiments, the detecting the nucleic acid sequence comprises quantitative polymerase chain reaction (qPCR), nucleic acid sequencing, or hybridization to a nucleic acid array. In some embodiments, the one or more POR histological factors comprises operative visceral adipose tissue (VAT) quality, wherein the operative VAT is defined by a reduced radiodensity. In some embodiments, the increased radiodensity comprises a VAT radiodensity of at least 0.631 HU. In some embodiments, the increased radiodensity comprises a VAT radiodensity of ranging from 0.631-0.926 HU. In some embodiments, the increased radiodensity comprises a VAT:SAT radiodensity of at least 5.559. In some embodiments, the increased radiodensity comprises a VAT:SAT radiodensity of ranging from 5.56-26. In some embodiments, the one or more POR histological factors comprises operative visceral adipose tissue (VAT) quality. In some embodiments, the likelihood of the POR is high relative to the control subject based, at least in part, on the one or more POR histological factors detected in the subject, wherein the one or more POR histological factors comprises the operative VAT quality, wherein operative VAT quality is defined by an increased radiodensity. In some embodiments, the therapeutic agent comprises an inhibitor or tumor necrosis factor alpha (TNFα), an inhibitor of Interleukin-23 (IL23), an inhibitor of integrin, an inhibitor of S1PR (Sphingosine-1-phosphate receptor), an inhibitor of TNF superfamily member 15 (TL1A), or an inhibitor of Janus kinase 2 (JAK), or any combination thereof. In some embodiments, the inhibitor of tumor necrosis factor alpha (TNFα) comprises adalimumab, infliximab, golimumab, certolizumab, or etanercept, or any combination thereof. In some embodiments, the inhibitor of IL23 comprises ustekinumab, guselkumab, risankizumab, brazikumab, mirikizumab, tildrakizumab, or briakinumab, or any combination thereof. In some embodiments, the inhibitor of integrin comprises etrolizumab, vedolizumab, natalizumab, or ontamalimab, or any combination. In some embodiments, the inhibitor of S1PR comprises fingolimod, siponimod, etrasimod, ozanimod, ponesimod, amiselimod, ceralifimod, or mocravimod, or any combination thereof. In some embodiments, the inhibitor of TLIA comprises tulisokibart, RVT-3101/PF-06480605, or duvakitug, or any combination thereof. In some embodiments, the inhibitor of JAK comprises upadacitinib, filgotinib, tofacitinib, abrocitinib, baricitinib, or any combination thereof. In some embodiments, the method comprises administering to the subject another therapeutic agent comprising an antagonist of interleukin 21 (IL-21). In some embodiments, the antagonist of IL-21 comprises an antibody, or an antigen-binding fragment thereof. In some embodiments, the one or more POR genetic factors comprises a single nucleotide polymorphism at a polymorphism position comprising rs6811007 or a proxy polymorphism position in linkage disequilibrium therewith as determined with an r² of at least 0.85, or a combination thereof. In some embodiments, the one or more POR genetic factors comprises a single nucleotide polymorphism at a polymorphism position comprising rs1533236, or a proxy polymorphism position in linkage disequilibrium therewith as determined with an r² of at least 0.85, or a combination thereof. In some embodiments, the one or more POR genetic factors comprises a single nucleotide polymorphism at a polymorphism position comprising rs6811007 or a proxy polymorphism position in linkage disequilibrium therewith as determined with an r² of at least 0.85, or a combination thereof; a single nucleotide polymorphism at a polymorphism position comprising rs1533236, or a proxy polymorphism position in linkage disequilibrium therewith as determined with an r² of at least 0.85, or a combination thereof, or both. In some embodiments, the polymorphism position comprising rs1533236 comprises an absence of an adenosine (“A”).

In certain aspects, disclosed herein is a method of enriching a target nucleic acid in a sample, the method comprising: providing a plurality of target nucleic acid sequences obtained from a biological sample from a subject with Crohn's disease (CD); bringing a fluid reaction formulation comprising a plurality of synthetic oligonucleotide molecules in contact with the plurality of target nucleic acid sequences, wherein each synthetic oligonucleotide molecule of the plurality of synthetic oligonucleotide molecules is complementary to at least one target nucleic acid sequence of the plurality of target nucleic acid sequences; hybridizing the plurality of synthetic oligonucleotide molecules to the plurality of synthetic oligonucleotide molecules; amplifying the plurality of target nucleic acid sequences hybridized to the plurality of synthetic oligonucleotide molecules in (c), thereby enriching the plurality of target nucleic acid sequences in the fluid reaction formulation; and detecting the plurality of target nucleic acid sequences that were enriched in (d), wherein the detecting in (e) of the plurality of target nucleic acid sequences is indicative of a high likelihood of post-operative prophylaxis persistence (“POPP”), as compared with a control subject._In some embodiments, the method comprises detecting an absence of a polymorphism comprising an adenosine (“A”) at rs6811007, or a proxy polymorphism position in linkage disequilibrium therewith as determined with an r² of at least 0.85, or a combination thereof, in the plurality of target nucleic acid sequences.

In certain aspects, disclosed herein is a method of enriching a target nucleic acid in a sample, the method comprising: providing a plurality of target nucleic acid sequences obtained from a biological sample from a subject with Crohn's disease (CD); bringing a fluid reaction formulation comprising a plurality of synthetic oligonucleotide molecules in contact with the plurality of target nucleic acid sequences, wherein each synthetic oligonucleotide molecule of the plurality of synthetic oligonucleotide molecules is complementary to at least one target nucleic acid sequence of the plurality of target nucleic acid sequences; hybridizing the plurality of synthetic oligonucleotide molecules to the plurality of synthetic oligonucleotide molecules; amplifying the plurality of target nucleic acid sequences hybridized to the plurality of synthetic oligonucleotide molecules in (c), thereby enriching the plurality of target nucleic acid sequences in the fluid reaction formulation; and detecting the plurality of target nucleic acid sequences that were enriched in (d), wherein the detecting in (e) of the plurality of target nucleic acid sequences is indicative of a high likelihood of post-operative recurrence (“POR”) as compared with a control subject. In some embodiments, the method comprises detecting a presence of a polymorphism comprising an adenosine (“A”) at rs6811007, or a proxy polymorphism position in linkage disequilibrium therewith as determined with an r² of at least 0.85, or a combination thereof, in the plurality of target nucleic acid sequences. In some embodiments, the control subject has CD and has undergone a surgical resection to treat the CD. In some embodiments, the plurality of synthetic oligonucleotide molecules comprises a detectable label. In some embodiments, the detectable label comprises a fluorescent label, a radioactive label, an enzyme, a chemiluminescent tag, or a colorimetric tag. In some embodiments, detecting the plurality of target nucleic acid sequences comprises performing quantitative polymerase chain reaction (qPCR). In some embodiments, the method comprises measuring one or more serological markers in a second sample obtained from the subject. In some embodiments, the measuring the one or more serological markers comprises introducing a detection agent configured to bind ANCA, ASCA IgA, ASCA IgG to the second sample under conditions sufficient to detect the ANCA, ASCA IgA, ASCA IgG, or combinations thereof, in the second sample. In some embodiments, the measuring the one or more serological markers in the second sample comprises performing enzyme-linked immunosorbent assay (ELISA). In some embodiments, the method comprises analyzing a third sample obtained from the subject for operative visceral adipose tissue (VAT) quality, wherein the operative VAT is defined by a reduced radiodensity. In some embodiments, the increased radiodensity comprises a VAT radiodensity of at least 0.631 HU. In some embodiments, the increased radiodensity comprises a VAT radiodensity of ranging from 0.631-0.926 HU. In some embodiments, the increased radiodensity comprises a VAT:SAT radiodensity of at least 5.559. In some embodiments, the increased radiodensity comprises a VAT:SAT radiodensity of ranging from 5.56-26.

In certain disclosed herein is a computing platform for predicting a post-operative prophylaxis persistence (“POPP”) in a subject, the platform comprising: a genotyping device for determining genotype data of a subject with Crohn's disease (CD), wherein the subject is receiving a treatment for the CD comprising a therapeutic agent; and one or more computer processors operably coupled to the genotyping device, wherein the one or more computer processors are individually or collectively configured to perform operations comprising: receiving the genotype data from the genotyping device; and identifying, in the genotype data, a genotype that is predictive of the POPP. In some embodiments, the method comprises: determining a positive result based on the genotype identified in (ii), wherein the positive result is indicative that the treatment of the CD exhibits a high likelihood to induce POPP in the subject, relative to a control sample obtained from a control subject with CD that has undergone a surgical recission to treat the CD. In some embodiments, the method comprises detecting an absence of a polymorphism comprising an adenosine (“A”) at rs6811007, or a proxy polymorphism in linkage disequilibrium therewith as determined with an r² of at least 0.85, or a combination thereof. In some embodiments, a high likelihood of comprises a likelihood of at least about 55%, 60%, 65%, 70%, or 75%. In some embodiments, the operations further comprise receiving POPP marker data for the subject, wherein the POPP marker data comprises POPP clinical factor data, POPP serological factor data, POPP genetic factor data, or any combination thereof. In some embodiments, the POPP clinical factor data comprises an active smoker status, disease location, a failure of a therapeutic agent to treat the CD, a resection length of operation, or a lymph node granuloma, or any combination thereof. In some embodiments, the operations further comprise identifying the subject as having a low likelihood of the POPP, wherein the POPP clinical factor data for the subject comprises the active smoker status. In some embodiments, the active smoker status is defined by smoking at least one cigarette per week. In some embodiments, the operations further comprise identifying a high likelihood that the therapeutic agent exhibits the POPP in treating the CD in the subject, wherein the POPP clinical factor data for the subject comprises the disease location in an ileal region of an intestine of the subject, wherein the high likelihood is relative to a control sample obtained from a control subject with CD that has undergone a surgical recission to treat the CD. In some embodiments, the operations further comprise identifying a high likelihood that the therapeutic agent exhibits the POPP in treating the CD in the subject based, wherein the POPP clinical factor data comprises the disease location in an ileocolonic region of an intestine of the subject, wherein the high likelihood is relative to a control sample obtained from a control subject with CD that has undergone a surgical recission to treat the CD. In some embodiments, the operations further comprise identifying a low likelihood that the therapeutic agent exhibits the POPP in treating the CD in the subject based, wherein the POPP clinical factor data comprises the lymph node granuloma, wherein the low likelihood is relative to a control sample obtained from a control subject with CD that has undergone a surgical recission to treat the CD. In some embodiments, the operations further comprise identifying a low likelihood that the therapeutic agent exhibits the POPP in treating the CD in the subject based, wherein the POPP clinical factors comprises the failure of the therapeutic agent for treatment of the CD in the subject, wherein the low likelihood is relative to a control sample obtained from a control subject with CD that has undergone a surgical recission to treat the CD. In some embodiments, the therapeutic agent comprises a steroid, an immunomodulator, 5-aminosalicylate, or combinations thereof. In some embodiments, the therapeutic agent comprises one or more biologic therapeutic agents for treatment of the CD. In some embodiments, the one or more biologic therapeutic agents for treatment of the CD comprises two or more biologic therapeutic agents. In some embodiments, the one or more biologic therapeutic agents for treatment of the CD comprises three or more biologic therapeutic agents. In some embodiments, the failure of the therapeutic agent is a recurrence or flare up of the CD following successful remission of the CD following administration of the therapeutic agent to the subject. In some embodiments, the operations further comprise receiving the one or more POPP clinical factors from the subject directly, or receiving medical records of the subject indirectly. In some embodiments, the POPP serological factor data comprises ANCA seronegativity, ASCA IgA seronegativity, or ASCA IgG seronegativity, or any combination thereof. In some embodiments, the operations further comprise identifying the subject as having seropositivity or seronegativity by calculating a percentage of a reference value from pooled sera obtained from a patient population diagnosed with CD. In some embodiments, the operations further comprise identifying a high likelihood that the therapeutic agent exhibits the POPP, wherein the POPP serological factor data comprises ASCA seropositivity, wherein the high likelihood is relative to a control sample obtained from a control subject with CD that has undergone a surgical recission to treat the CD. In some embodiments, the operations further comprise identifying a high likelihood that the therapeutic agent exhibits the POPP, wherein the POPP serological factor data comprises ANCA seronegativity, wherein the high likelihood is relative to a control sample obtained from a control subject with CD that has undergone a surgical recission to treat the CD. In some embodiments, the operations further comprise identifying a high likelihood that the therapeutic agent exhibits the POPP, wherein the POPP serological factor data comprises ASCA seropositivity and ANCA seronegativity, wherein the high likelihood is relative to a control sample obtained from a control subject with CD that has undergone a surgical recission to treat the CD. In some embodiments, the operations further comprise identifying a high likelihood that the therapeutic agent exhibits the POPP, wherein the POPP histological factor data for the subject comprises a operative visceral adipose tissue (VAT) quality, as defined by a reduced radiodensity. In some embodiments, the reduced radiodensity comprises a VAT radiodensity of less than or equal to about 0.631 Hounsfield unit (HU). In some embodiments, the reduced radiodensity comprises a VAT radiodensity of about 0.43 to about 0.7 HU. In some embodiments, the reduced radiodensity comprises a ratio of VAT to SAT radiodensity of up to 5.559. In some embodiments, the reduced radiodensity comprises a ratio of VAT to SAT radiodensity ranging from 1.18-6. In some embodiments, the genotyping device comprises a nucleic acid sequencer, a nucleic acid array, or a quantitative polymerase chain reaction (qPCR) machine. In some embodiments, the method comprise detecting one or more POPP genetic factors comprises a single nucleotide polymorphism at a polymorphism position comprising rs6811007 or a proxy polymorphism position in linkage disequilibrium therewith as determined with an r² of at least 0.85, or a combination thereof. In some embodiments, the method comprise detecting one or more POPP genetic factors comprises a single nucleotide polymorphism at a polymorphism position comprising rs1533236, or a proxy polymorphism position in linkage disequilibrium therewith as determined with an r² of at least 0.85, or a combination thereof. In some embodiments, In some embodiments, the method comprise detecting one or more POPP genetic factors comprises a single nucleotide polymorphism at a polymorphism position comprising rs6811007 or a proxy polymorphism position in linkage disequilibrium therewith as determined with an r² of at least 0.85, or a combination thereof; a single nucleotide polymorphism at a polymorphism position comprising rs1533236, or a proxy polymorphism position in linkage disequilibrium therewith as determined with an r² of at least 0.85, or a combination thereof, or both. In some embodiments, the polymorphism position comprising rs1533236 comprises an absence of an adenosine (“A”).

In certain aspects, disclosed herein is a computing platform for predicting a post-operative recurrence (“POR”) in a subject, the platform comprising: a genotyping device for determining genotype data of a subject with Crohn's disease (CD), wherein the subject is receiving a treatment for the CD comprising a therapeutic agent; and one or more computer processors operably coupled to the genotyping device, wherein the one or more computer processors are individually or collectively configured to perform operations comprising: receiving the genotype data from the genotyping device; and identifying, in the genotype data, a predetermined genotype that is predictive of the POR. In some embodiments, the method comprises determining a positive result based on the genotype identified in (ii), wherein the positive result is that the subject exhibits a high likelihood of the POR, relative to a control sample obtained from a control subject with CD that has undergone a surgical recission to treat the CD. In some embodiments, the genotype comprises a presence of a polymorphism comprising an adenosine (“A”) at rs6811007, or a proxy polymorphism in linkage disequilibrium therewith as determined with an r² of at least 0.85, or a combination thereof. In some embodiments, the positive result is further indicative that the subject will not respond to the treatment comprising the therapeutic agent. In some embodiments, operations further comprise receiving POR marker data for the subject, wherein the POR marker data comprises POR clinical factor data, POR serological factor data, POR genetic factor data, or POR histological factor data, or any combination thereof. In some embodiments, the POR clinical factor data comprises an active smoker status, disease location, a failure of a therapeutic agent to treat the CD, a resection length of operation, or a lymph node granuloma, or any combination thereof. In some embodiments, the operations further comprise identifying the subject as having a high likelihood of the POR, wherein the one or more POR clinical factors detected in the subject comprise the active smoker status, wherein the high likelihood is relative to a control sample obtained from a control subject with CD that has undergone a surgical recission to treat the CD. In some embodiments, the operations further comprise identifying the subject as having a high likelihood of the POR wherein the POR clinical factor data detected comprises the lymph node granuloma, wherein the high likelihood is relative to a control sample obtained from a control subject with CD that has undergone a surgical recission to treat the CD. In some embodiments, the operations further comprise identifying the subject as having a high likelihood of the POR, wherein the POR clinical factor data comprises the disease location in an ileocolonic region of an intestine of the subject, wherein the high likelihood is relative to a control sample obtained from a control subject with CD that has undergone a surgical recission to treat the CD. In some embodiments, the operations further comprise identifying the subject as having a low likelihood of the POR, wherein the POR clinical factor data comprises the disease location in an ileal region of an intestine of the subject, wherein the low likelihood is relative to a control sample obtained from a control subject with CD that has undergone a surgical recission to treat the CD. In some embodiments, the operations further comprise identifying the subject as having a high likelihood of the POR wherein the POR clinical factor data comprises the failure of the therapeutic agent for treatment of the CD, wherein the high likelihood is relative to a control sample obtained from a control subject with CD that has undergone a surgical recission to treat the CD. In some embodiments, the therapeutic agent comprises an immunomodulator, 5-aminosalicylate, or combinations thereof. In some embodiments, the therapeutic agent comprises one or more biologic therapeutic agents. In some embodiments, the one or more biologic therapeutic agents comprises two or more biologic therapeutic agents for the treatment of the CD. In some embodiments, the one or more biologic therapeutic agent comprises three or more biologic therapeutic agents for the treatment of the CD. In some embodiments, the operations further comprise identifying the subject as having a high likelihood of the POR, wherein the POR serological factor data for the subject comprises ANCA seropositivity, ASCA IgA seropositivity, or ASCA IgG seropositivity, or any combination thereof, wherein the high likelihood is relative to a control sample obtained from a control subject with CD that has undergone a surgical recission to treat the CD. In some embodiments, the operations further comprise identifying the subject as having a high likelihood of the POR, wherein the POR serological factor data comprises ASCA seropositivity, wherein the high likelihood is relative to a control sample obtained from a control subject with CD that has undergone a surgical recission to treat the CD. In some embodiments, the operations further comprise identifying the subject as having a high likelihood of the POR, wherein the POR clinical factor data comprises ANCA seronegativity, wherein the high likelihood is relative to a control sample obtained from a control subject with CD that has undergone a surgical recission to treat the CD. In some embodiments, the operations further comprise identifying the subject as having a high likelihood of the POR, wherein the POR clinical factor data comprises ASCA seropositivity and ANCA seronegativity, wherein the high likelihood is relative to a control sample obtained from a control subject with CD that has undergone a surgical recission to treat the CD. In some embodiments, the POR histological factor data comprises an operative visceral adipose tissue (VAT) quality. In some embodiments, the operations further comprise identifying the subject as having a high likelihood of the POR, wherein the operative VAT quality is defined by an increased radiodensity. In some embodiments, the increased radiodensity comprises a VAT radiodensity of at least 0.631 HU. In some embodiments, the increased radiodensity comprises a VAT radiodensity of ranging from 0.631-0.926 HU. In some embodiments, the increased radiodensity comprises a VAT:SAT radiodensity of at least 5.559. In some embodiments, the reduced radiodensity comprises a VAT:SAT radiodensity of ranging from 5.56-26. In some embodiments, the genotype comprises a presence of a polymorphism the one or more POR genetic factors comprises a single nucleotide polymorphism at a polymorphism position comprising rs6811007 or a proxy polymorphism position in linkage disequilibrium therewith as determined with an r² of at least 0.85, or a combination thereof. In some embodiments, the genotype comprises a presence of a polymorphism position comprising rs1533236, or a proxy polymorphism position in linkage disequilibrium therewith as determined with an r² of at least 0.85, or a combination thereof. In some embodiments, the genotype comprises a presence of a polymorphism at a polymorphism position comprising rs6811007 or a proxy polymorphism position in linkage disequilibrium therewith as determined with an r² of at least 0.85, or a combination thereof; a single nucleotide polymorphism at a polymorphism position comprising rs1533236, or a proxy polymorphism position in linkage disequilibrium therewith as determined with an r² of at least 0.85, or a combination thereof; or both. In some embodiments, the polymorphism position comprising rs1533236 comprises an absence of an adenosine (“A”).

In certain aspects, disclosed herein is a kit for predicting a post-operative prophylaxis persistence (“POPP”) in a subject having CD, the kit comprising: a detection agent configured to bind or hybridize to a polymorphism comprising an adenosine (“A”) at rs6811007 or a proxy polymorphism in linkage disequilibrium (LD) therewith as determined with an r² of at least 0.85, or a combination thereof; and instructions for use of the detection agent to detect an absence of the polymorphism or the proxy polymorphism in LD therewith in a sample obtained from the subject. In some embodiments, the method comprises: a second detection agent configured to bind ANCA, ASCA IgA, ASCA IgG, or combinations thereof; and instructions for use of the second detection agent to detect seronegative or seropositivity of the ANCA, the ASCA IgA, the ASCA IgG, or any combinations thereof in a sample from the subject. In some embodiments, the instructions further comprise instructions for predicting the POPP in the subject. In certain aspects, disclosed herein is kit for predicting a post-operative recurrence (“POR”) in a subject having CD, the kit comprising: a detection agent configured to bind or hybridize to a polymorphism comprising an adenosine (“A”) at rs6811007, or a proxy polymorphism in linkage disequilibrium (LD) therewith as determined with an r² of at least 0.85, or a combination thereof; and instructions for use of the detection agent to detect a presence of the polymorphism or the proxy polymorphism in LD therewith in a sample obtained from the subject. In some embodiments, the method comprises: a second detection agent configured to bind ANCA, ASCA IgA, ASCA IgG in a sample from the subject; and instructions for use of the second detection agent to detect seronegative or seropositivity of the ANCA, the ASCA IgA, the ASCA IgG, or any combinations thereof in a sample from the subject. In some embodiments, the instructions further comprise instructions for predicting the POR in the subject. In some embodiments, a detection agent configured to bind or hybridize to a polymorphism a single nucleotide polymorphism at a polymorphism position comprising rs6811007 or a proxy polymorphism position in linkage disequilibrium therewith as determined with an r² of at least 0.85, or a combination thereof. a detection agent configured to bind or hybridize to a polymorphism position comprising rs1533236, or a proxy polymorphism position in linkage disequilibrium therewith as determined with an r² of at least 0.85, or a combination thereof. a detection agent configured to bind or hybridize to a polymorphism position comprising rs6811007 or a proxy polymorphism position in linkage disequilibrium therewith as determined with an r² of at least 0.85, or a combination thereof; a single nucleotide polymorphism at a polymorphism position comprising rs1533236, or a proxy polymorphism position in linkage disequilibrium therewith as determined with an r² of at least 0.85, or a combination thereof; or both. In some embodiments, the polymorphism position comprising rs1533236 comprises an absence of an adenosine (“A”).

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a computer system that is programmed or otherwise configured to implement methods provided herein.

FIG. 2A depicts the preoperative medications of the subjects.

FIG. 2B depicts the post-operative prophylaxis of the subjects.

FIG. 2C depicts the time to prophylaxis failure of the subjects.

FIG. 2D depicts the reasons for prophylaxis failure.

FIG. 2E depicts clinical factors associated with prophylaxis failure.

FIG. 3 depicts GWAS catalog hits on chromosome 4 for prophylaxis.

FIG. 4 depicts an example of radiographic visceral adipose tissue (VAT) (orange) and SAT (purple) metrics extracted from CT images. Of note, the lower the radiodensity is suggested to reflect poorer quality of fat.

FIG. 5 depicts an adjusted survival curve comparing post-operative prophylaxis persistence for TNF alpha inhibitors vs. non-TNF alpha inhibitors (anti-integrin, n=20; anti-IL 12/23, n=40). aHR: adjusted hazards ratio, CI: confidence interval; ^{β}Adjusted for age, sex, disease duration, smoking status, and # of prior biologic exposure.

FIG. 6 illustrates the inclusion/exclusion of subjects for the study described in Example 6.

FIG. 7 provides a flowchart illustrating the inclusion and exclusion criteria in Example 7.

### DETAILED DESCRIPTION

Provided herein are methods for the treatment, analysis, characterization, and diagnosis of a disease or condition. Also provided are methods of selection for treatment of a disease or condition. In certain aspects, described herein is a method of treating Crohn's disease (CD) in a subject. In certain aspects, described herein is a method of determining prophylactic persistence of therapeutic agent in treating Crohn's disease (CD) in a subject that has undergone operation. In certain aspects, described herein is a method of determining the prognosis of post-operative recurrence (“POR”) of Crohn's disease (CD) in a subject.

Post-operative recurrence (POR) occurs in ~30% of patients on post-operative prophylaxis. Drug persistence may serve as a real-world proxy for safety and efficacy and an important consideration for therapeutic positioning, but factors involved in post-operative prophylaxis persistence is unknown. Described herein are clinical, serologic, and genetic factors associated with post-operative prophylaxis persistence (POPP) or POR.

In some embodiments, described herein is a method of treating CD in a subject. In some embodiments, the method comprises administering to the subject a therapeutically effective amount of a therapeutic agent for CD, wherein one or more post-operative prophylaxis persistence (“POPP”) markers were detected in the subject. In some embodiments, the method comprising: administering to the subject a therapeutically effective amount of a therapeutic agent, wherein one or more post-operative recurrence (“POR”) markers were detected in the subject.

In certain aspects, described herein is a method of determining prophylactic persistence of therapeutic agent in treating Crohn's disease (CD) in a subject that has undergone operation. In some embodiments, the method comprises detecting one or more post-operative prophylaxis persistence (“POPP”) markers in the subject. In some embodiments, the method comprises determining the therapeutic agent to have the prophylactic persistence in treating the CD in the subject based, at least in part, on the one or more POPP markers detected in the subject, wherein the CD is post-operative.

In certain aspects, described herein is a method of determining the prognosis of post-operative recurrence (“POR”) of Crohn's disease (CD) in a subject. In some embodiments, the method comprises detecting one or more POR markers in the subject. In some embodiments the method comprises determining the subject to have increased likelihood of POR based, at least in part on the one or more POR markers detected in the subject, wherein the increased likelihood of POR is compared with a control subject having the CD, wherein the control subject does not have the one or more POR markers.

### Post-Operative Prophylaxis Persistence Markers

In certain aspects, the methods and compositions described herein comprise detecting one or more post-operative prophylaxis persistence (POPP) markers in the subject. In some embodiments, the one or more POPP markers comprises one or more POPP clinical factors, one or more POPP serological factors, one or more POPP genetic factors, or any combination thereof.

### Clinical Factors

In some embodiments, the one or more POPP clinical factors comprises one or more selected from the group consisting of active smoking, diseased organ (ileocolonic vs. ileal disease), number of prior treatment failures. number of prior biologic therapy failures, resection length of operation, and or lymph node granuloma, or any combination thereof. In some embodiments, the clinical factors are determined by inquiry of the subject directly, or reviewing the medical records of the subject. In some embodiments, the clinical factors are determined by inquiry of the subject directly. In some embodiments, the one or more clinical factors are determined by reviewing the medical records of the subject.

In some embodiments, the method comprise identifying the subject as having the high likelihood of the POPP, wherein if the one or more POPP clinical factors detected in the subject comprises exhibits a disease location in the ileal region of an intestine of the subject. In some embodiments, the one or more POPP clinical factors detected in the subject comprises a disease location in the ileocolonic region of an intestine of the subject.

In some embodiments, the POPP clinical factor comprises identifying the subject as an active smoker. An active smoker may be defined as a subject who smokes at least one cigarette per week. In some embodiments, the subject, has a low likelihood of POPP if they are an active smoker.

In some embodiments, the one or more POPP clinical factors detected in the subject comprises lymph node granuloma. In some embodiments, the subject is classified as having a low likelihood of POPP if the subject exhibits lymph node granuloma.

In some embodiments, the subject is identified as having a low likelihood of POPP if the subject has exhibited one or more prior treatment failures, the prior treatments comprising a steroid, an immunomodulator, 5-aminosalicylate, or combinations thereof. The prior treatment may comprise a steroid. The prior treatment may comprise an immunomodulator. The prior treatment may comprise a 5-aminosalicylate.

In some embodiments, the POPP clinical factor comprises the subject exhibiting one or more prior biologic failures. In some embodiments, the subject has a low likelihood of POPP if the subject has exhibited one or more prior biologic failures. In some embodiments, the subject has a low likelihood of POPP if the subject has exhibited two or more prior biologic failures. In some embodiments, the subject has a low likelihood of POPP if the subject has exhibited three or more prior biologic failures. A biologic failure may comprise a recurrence or flare up of CD following successful remission of CD following administration of a biologic therapeutic agent to the subject.

In some embodiments, the methods comprise. e determining the likelihood of the POPP comprises identifying the subject as having a high likelihood of the POPP, wherein the one or more POPP clinical factors detected in the subject comprises operative visceral adipose tissue (VAT) quality, as defined by a reduced radiodensity.

In some embodiments, the reduced radiodensity comprises a radiodensity of less than or equal to about 0.631 HU. In some embodiments, the reduced radiodensity comprises a radiodensity of less than or equal to about 0.1, 0.2, 0.3, 0.4, 0.5, 0.6, or 0.7 HU. In some embodiments, the reduced radiodensity comprises a VAT radiodensity of about 0.43 HU to about 0.7 HU. In some embodiments, the reduced radiodensity comprises a VAT radiodensity of about 0.1 HU to about 0.7 HU, about 0.2 HU to about 0.7 HU, about 0.3 HU to about 0.7 HU, about 0.4 HU to about 0.7 HU, about 0.5 HU to about 0.7 HU, or about 0.6 HU to about 0.7 HU. In some embodiments, the reduced radiodensity comprises a VAT radiodensity of about 0.1 HU to about 0.7 HU, about 0.1 HU to about 0.6 HU, about 0.1 HU to about 0.5 HU, about 0.1 HU to about 0.4 HU, about 0.1 HU to about 0.3 HU, or about 0.1 HU to about 0.2 HU.

In some embodiments, the reduced ratio density comprises a ratio of VAT to subcutaneous adipose tissue (SAT) radiodensity (VAT:SAT) of up to 5.559. In some embodiments, the reduced radiodensity comprises a VAT:SAT radiodensity ratio of up to about 1, about 1.5, about 2, about 2.5, about 3, about 3.5, about 4, about 4.5, about 5, about 5.5, or about 6. In some embodiments, the reduced radiodensity comprises a VAT:SAT radiodensity ratio between about 1.18 to about 6. In some embodiments, the reduced radiodensity comprises a VAT:SAT radiodensity ratio between about 1 to about 6, about 1.5 to about 6, about 2 to about 6, about 2.5 to about 6, about 3 to about 6, about 3.5 to about 6, about 4 to about 6, about 4.5 to about 6, about 5 to about 6, or about 5.5 to about 6. In some embodiments, the reduced radiodensity comprises a VAT:SAT radiodensity ratio of between about 1 to about 1.5, about 1 to about 2, about 1 to about 2.5, about 1 to about 3, about 1 to about 3.5, about 1 to about 4, about 1 to about 4.5, about 1 to about 5, about 1 to about 5.5, or about 1 to about 6.

### Serological Factors

In some embodiments, the one or more POPP markers comprises a serological factor. In some embodiments, the one or more POPP serological factors comprises one or more selected from the group consisting of ANCA seronegativity, ASCA IgA seronegativity, or ASCA IgG seronegativity, or any combination thereof.

In some embodiments, seropositivity or seronegativity is measured by calculating a percentage of a reference value from pooled sera obtained from a patient population diagnosed with CD.

In some embodiments, the methods comprise identifying the subject as having a high likelihood of POPP, if the subject exhibits ASCA seropositivity. In some embodiments, the methods comprise identifying the subject as having a high likelihood of POPP if the subject exhibits ANCA seronegativity. In some embodiments, the methods comprise identifying the subject as having a high likelihood of POPP if the subject exhibits ASCA seropositivity and ANCA seronegativity.

### Genetic Factors

In some embodiments, the one or more POPP genetic factors comprises an absence of an allele comprising an adenosine (“A”) at a polymorphism position comprising rs6811007, or a proxy polymorphism position in linkage disequilibrium therewith. In some embodiments, the one or more POPP genetic factors is homozygous for an allele at a polymorphism position disclosed herein. In some embodiments, the one or more POPP genetic factors is heterozygous for an allele at a polymorphism position disclosed herein. For example, the one or more POPP genetic factors may be an absence of two copies of the “A” allele at rs6811007 or a proxy polymorphism therewith. In some embodiments, the one or more POPP genetic factors comprises an absence of an allele at a polymorphism position listed in Table 3 or Table 4. The genotypes disclosed herein are, in some cases, a haplotype. In some instances, the genotype comprises a particular polymorphism, a polymorphism in linkage disequilibrium (LD) therewith, or a combination thereof. In some cases, LD is defined by an r² of at least or about 0.70, 0.75, 0.80, 0.85, 0.90, or 1.0. The genotypes disclosed herein can comprise at least or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more polymorphisms. In some embodiments, a high likelihood of POR is at least about 55%, 60%, 65%, 70%, 75%, 80%, 85%, 90%, 95% or 99%.

In some embodiments, the one or more POPP genetic factors comprises a polymorphism position comprising rs1533236. In some embodiments, the one or more POPP genetic factors comprises an absence of an allele comprising an adenosine (“A”) at a polymorphism position comprising rs1533236, or a proxy polymorphism position in linkage disequilibrium therewith. In some embodiments, the one or more POPP genetic factors is homozygous for an allele at a polymorphism position disclosed herein. In some embodiments, the one or more POPP genetic factors is heterozygous for an allele at a polymorphism position disclosed herein. For example, the one or more POPP genetic factors may be an absence of two copies of the “A” allele at rs6811007 or a proxy polymorphism therewith. In some embodiments, the one or more POPP genetic factors comprises an absence of an allele at a polymorphism position listed in Table 3 or Table 4. The genotypes disclosed herein are, in some cases, a haplotype. In some instances, the genotype comprises a particular polymorphism, a polymorphism in linkage disequilibrium (LD) therewith, or a combination thereof. In some cases, LD is defined by an r² of at least or about 0.70, 0.75, 0.80, 0.85, 0.90, or 1.0. The genotypes disclosed herein can comprise at least or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more polymorphisms. In some embodiments, a high likelihood of POR is at least about 55%, 60%, 65%, 70%, 75%, 80%, 85%, 90%, 95% or 99%.

Disclosed herein are genotypes related to risk of POPP or POR in a subject with CD. In some embodiments, the genotypes that may be detected in a sample obtained from a subject by analyzing the genetic material in the sample. In some embodiments, the subject may be human. In some embodiments, the genetic material is obtained from a subject having a disease or condition disclosed herein. In some cases, the genetic material is obtained from blood, serum, plasma, sweat, hair, tears, urine, and other techniques. In some cases, the genetic material is obtained from a biopsy, e.g., from the intestinal track of the subject.

The genotypes described herein are detected using suitable genotyping devices (e.g., array, sequencing). In some embodiments, a sample is obtained from the subject or patient indirectly or directly. In some embodiments, the sample may be obtained by the subject. In other instances, the sample may be obtained by a healthcare professional, such as a nurse or physician. The sample may be derived from virtually any biological fluid or tissue containing genetic information, such as blood.

The genotypes of the present disclosure comprise genetic material that is deoxyribonucleic acid (DNA). In some embodiments, the genotype comprises a denatured DNA molecule or fragment thereof. In some embodiments, the genotype comprises DNA selected from: genomic DNA, viral DNA, mitochondrial DNA, plasmid DNA, amplified DNA, circular DNA, circulating DNA, cell-free DNA, or exosomal DNA. In some embodiments, the DNA is single-stranded DNA (ssDNA), double-stranded DNA, denaturing double-stranded DNA, synthetic DNA, and combinations thereof. The circular DNA may be cleaved or fragmented.

The genotypes disclosed herein comprise at least one polymorphism located at a gene or genetic locus described herein. In some embodiments, the gene or genetic locus is IL21|BBS12. The genotypes disclosed herein are, in some cases, a haplotype. In some embodiments, the genotype comprises a particular polymorphism, a polymorphism in linkage disequilibrium (LD) therewith, or a combination thereof. In some cases, LD is defined by an r² of at least or about 0.70, 0.75, 0.80, 0.85, 0.90, or 1.0. The genotypes disclosed herein can comprise at least or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more polymorphisms.

The polymorphisms described herein can be a single nucleotide polymorphism, or an indel (insertion/deletion). In some embodiments, the polymorphism is an insertion or a deletion of at least one nucleobase (e.g., an indel). In some embodiments, the genotype may comprise a copy number variation (CNV), which is a variation in a number of a nucleic acid sequence between individuals in a given population. In some embodiments, the CNV comprises at least or about two, three, four, five, six, seven, eight, nine, ten, twenty, thirty, forty or fifty nucleic acid molecules. In some embodiments, the genotype is heterozygous. In some embodiments, the genotype is homozygous.

### Post-Operative Recurrence Markers

In some embodiments, the one or more POR markers comprise one or more POR clinical factors, one or more POR serological factors, one or more POR genetic factors, or one or more POR histological factors, or any combination thereof.

In some embodiments, determining a likelihood of POR comprises identifying the subject as having a high likelihood of if the subject exhibits the one or more POR clinical factors, one or more POR serological factors, one or more POR genetic factors, or one or more POR histological factors, or any combination thereof.

In some embodiments, the one or more POR clinical factors comprises an active smoking status, disease location (e.g., ileocolonic vs. ileal disease), number of prior treatment failures, number of prior biologic therapy failures, resection length of operation, and or lymph node granuloma, or any combination thereof. In some embodiments, the clinical factors are determined by inquiry of the subject directly, or reviewing the medical records of the subject. In some embodiments, the clinical factors are determined by inquiry of the subject directly. In some embodiments, the one or more clinical factors are determined by reviewing the medical records of the subject.

In some embodiments, the methods comprise identifying the subject as having the low likelihood of the POR, wherein if the one or more POR clinical factors detected in the subject comprises exhibits a disease location in the ileal region of an intestine of the subject. In some embodiments, the one or more POR clinical factors detected in the subject comprises a disease location in the ileocolonic region of an intestine of the subject.

In some embodiments, the POR clinical factor comprises identifying the subject as an active smoker. An active smoker may be defined as a subject who smokes at least one cigarette per week. In some embodiments, the subject, has a high likelihood of POR if they are an active smoker.

In some embodiments, the one or more POR clinical factors detected in the subject comprises lymph node granuloma. In some embodiments, the subject is classified as having a high likelihood of POPP if the subject exhibits lymph node granuloma.

In some embodiments, the subject is identified as having a high likelihood of POR if the subject has exhibited one or more prior treatment failures, the prior treatments comprising a steroid, an immunomodulator, 5-aminosalicylate, or combinations thereof. The prior treatment may comprise a steroid. The prior treatment may comprise an immunomodulator. The prior treatment may comprise a 5-aminosalicylate.

In some embodiments, the POR clinical factor comprises the subject exhibiting one or more prior biologic failures. In some embodiments, the subject has a high likelihood of POR if the subject has exhibited one or more prior biologic failures. In some embodiments, the subject has a high likelihood of POR if the subject has exhibited two or more prior biologic failures. In some embodiments, the subject has a high likelihood of POR if the subject has exhibited three or more prior biologic failures.

In some embodiments, the methods comprise determining the likelihood of the POR comprises identifying the subject as having a high likelihood of the POR, wherein the one or more POR clinical factors detected in the subject comprises operative visceral adipose tissue (VAT) quality. In some embodiments, the subject has a high likelihood of POR when the subject has an increased radiodensity.

In some embodiments, the increased radiodensity comprises a radiodensity of greater than or equal to about 0.631 Hounsfield unit (HU). In some embodiments, the increased radiodensity comprises a radiodensity greater than or equal to about 0.7 HU, 0.8 HU, 0.9 HU, 1.0 HU, or 1.1 HU. In some embodiments, the increased radiodensity comprises a VAT radiodensity of about 0.631 HU to about 0.926 HU. In some embodiments, the increased radiodensity comprises a VAT radiodensity of about 0.7 HU to about 0.8 HU, about 0.7 HU to about 0.9 HU, about 0.7 HU to about 1.0 HU, or about 0.7 HU to about 1.1 HU. In some embodiments, the increased radiodensity comprises a VAT radiodensity of about 0.7 HU to about 1.1 HU, about 0.8 HU to about 1.1 HU, about 0.9 HU to about 1.1 HU, or about 1.0 HU to about 1.1 HU.

In some embodiments, the increased ratio density comprises a ratio of VAT to subcutaneous adipose tissue (SAT) radiodensity (VAT:SAT) of at least 5.559. In some embodiments, the reduced radiodensity comprises a VAT:SAT radiodensity ratio of at least about 5.5, about 6, about 7, about 8, about 9, about 10, about 11, about 12, about 13, about 14, about 15, about 16, about 17, about 18, about 19, about 20, about 21, about 22, about 23, about 24 about 25 or about 26. In some embodiments, the reduced radiodensity comprises a VAT:SAT radiodensity ratio between about 5.56 to about 26. In some embodiments, the reduced radiodensity comprises a VAT:SAT radiodensity ratio between about 5.5 to about 26, about 6 to about 26, about 7 to about 26, about 8 to about 26, about 9 to about 26, about 10 to about 26, about 11 to about 26, about 12 to about 26, about 13 to about 26, about 14 to about 26, about 15 to about 26, about 16 to about 26, about 17 to about 26, about 18 to about 26, about 19 to about 26, about 20 to about 26, about 21 to about 26, about 22 to about 26, about 23 to about 26, about 24 to about 26, or about 25 to about 26.

In some embodiments, the one or more POR factor comprises a serological factor. In some embodiments, the one or more POR serological factors comprises one or more selected from the group consisting of ANCA seronegativity, ASCA IgA seronegativity, or ASCA IgG seronegativity, or any combination thereof. In some embodiments, seropositivity or seronegativity is measured by calculating a percentage of a reference value from pooled sera obtained from a patient population diagnosed with CD.

In some embodiments, the methods comprise identifying the subject as having a high likelihood of POR, if the subject exhibits ASCA seropositivity. In some embodiments, the methods comprise identifying the subject as having a high likelihood of POR if the subject exhibits ANCA seronegativity. In some embodiments, the methods comprise identifying the subject as having a high likelihood of POR if the subject exhibits ASCA seropositivity and ANCA seronegativity.

In some embodiments, the one or more POR genetic factors comprises an absence of an allele comprising an adenosine (“A”) at a polymorphism position comprising rs6811007, or a proxy polymorphism position in linkage disequilibrium therewith. In some embodiments, the one or more POR genetic factors comprises an absence of an allele at a polymorphism position listed in Table 3 or Table 4. In some embodiments, the one or more POR genetic factors is homozygous for an allele at a polymorphism position disclosed herein. In some embodiments, the one or more POR genetic factors is heterozygous for an allele at a polymorphism position disclosed herein. For example, the one or more POR genetic factors may be a presence of two copies of the “A” allele at rs6811007 or a proxy polymorphism therewith. The genotypes disclosed herein are, in some cases, a haplotype. In some instances, the genotype comprises a particular polymorphism, a polymorphism in linkage disequilibrium (LD) therewith, or a combination thereof. In some cases, LD is defined by an r² of at least or about 0.70, 0.75, 0.80, 0.85, 0.90, or 1.0. The genotypes disclosed herein can comprise at least or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more polymorphisms. In some embodiments, a high likelihood of POR is measured with a positive predictive value (PPV) of at least about 50, 55, 60, 65, 70, or 75%.

### Subject

The subject disclosed herein can be a mammal, such as for example a mouse, rat, guinea pig, rabbit, non-human primate, or farm animal. In some embodiments, the subject is human. In some embodiments, the subject is suffering from a symptom related to a disease or condition disclosed herein (e.g., abdominal pain, cramping, diarrhea, rectal bleeding, fever, weight loss, fatigue, loss of appetite, dehydration, and malnutrition, anemia, or ulcers). In some embodiments, the subject is a pediatric subject. For instance, a human subject may be 17 years old or younger. In some embodiments, the subject is an adult subject. For instance, a human subject may be between 18 and 64 years old. In some embodiments, the subject is an elderly subject. For instance, a human subject may be 65 years old or older. In some embodiments, the subject is female. In some embodiments, the subject is male.

In some embodiments, the subject is susceptible to, or is inflicted with, thiopurine toxicity, or a disease caused by thiopurine toxicity (such as pancreatitis or leukopenia). The subject may experience, or is suspected of experiencing, non-response or loss-of-response to a standard treatment (e.g., anti-tumor necrosis factor (TNF) therapy, anti-a4-b7 therapy (e.g., vedolizumab), anti-IL12p40 therapy (ustekinumab), Thalidomide, a glucocorticosteriod, or Cytoxan). In some embodiments, the subject may be currently receiving one or more of the standard therapies disclosed herein as a “first-line” therapy, such as the anti-TNF therapy and a glucocorticosteriods. In some embodiments, the subject is in need of a “second-line” therapy disclosed herein (e.g., anti-TL1A antibody) because the subject is either not responding to an induction of the standard therapy or has lost response to the standard therapy during a treatment course or regimen.

### Disease or Condition

In some embodiments, the disease or the condition is an inflammatory bowel disease, such as Crohn's disease (CD). In some embodiments, the Crohn's disease is moderate to severe Crohn's disease. In some cases, the CD is severe CD. The severe CD may result from inflammation that has led to the formation of scar tissue in the intestinal wall (fibrostenosis) and/or swelling. In some cases, the severe CD is characterized by the presence of fibrotic and/or inflammatory strictures. The strictures may be determined by computed tomography enterography (CTE), and magnetic resonance imaging enterography (MRE). The disease or condition may be characterized as refractory, which in some cases, means the disease is resistant to a standard treatment (e.g., anti-TNFα therapy). Non-limiting examples of standard treatment include glucocorticosteriods, anti-TNF therapy, anti-a4-b7 therapy (vedolizumab), anti-IL12p40 therapy (ustekinumab), Thalidomide, and Cytoxin.

### Methods

Methods disclosed herein comprise methods for detection of at least one POR marker or at least one POPP marker in a subject and methods of treating a subject. In some embodiments, the methods describe methods for characterizing the treatment of a subject. In some embodiments, the methods comprise methods of monitoring treatment. In some embodiments, the methods comprise methods of selecting a subject for treatment. In some embodiments, the subject has Crohn's Disease.

### Methods of Detection

In certain aspects, the methods described herein involve the detection of at least one POR marker or at least one POPP marker. In some embodiments, the at least one POR marker is a serological factor. In some embodiments, the at least one POR marker is a genetic factor. In some embodiments, the at least one POPP marker is a serological factor. In some embodiments, the at least one POPP marker is a genetic factor.

In some embodiments, the methods involve the detection of at least one POR marker or at least one POPP marker from a sample. In some embodiments, the genetic material is obtained from a subject having a disease or condition disclosed herein. In some cases, the genetic material is obtained from blood, serum, plasma, sweat, hair, tears, urine, and other techniques known by one of skill in the art. In some cases, the genetic material is obtained from a biopsy, e.g., from the intestinal track of the subject.

### Methods of Detecting a Genotype

Methods disclosed herein for detecting a genotype in a sample from a subject comprise analyzing the genetic material in the sample to detect at least one of a presence, an absence, and a quantity of a nucleic acid sequence encompassing the genotype of interest. In some embodiments, rs6811007 is detected using the methods described herein. In some embodiments, a polymorphism listed in Table 3 or Table 4 is detected using the methods described herein. In some embodiments, rs1533236 is detected using the methods described herein.

In some cases, the nucleic acid sequence comprises DNA. In some embodiments, the nucleic acid sequence comprises a denatured DNA molecule or fragment thereof. In some embodiments, the nucleic acid sequence comprises DNA selected from: genomic DNA, viral DNA, mitochondrial DNA, plasmid DNA, amplified DNA, circular DNA, circulating DNA, cell-free DNA, complementary DNA (cDNA), or exosomal DNA. In some embodiments, the DNA is single-stranded DNA (ssDNA), double-stranded DNA, denaturing double-stranded DNA, synthetic DNA, and combinations thereof. The circular DNA may be cleaved or fragmented. In some embodiments, the nucleic acid sequence comprises RNA. In some embodiments, the nucleic acid sequence comprises fragmented RNA. In some embodiments, the nucleic acid sequence comprises partially degraded RNA. In some embodiments, the nucleic acid sequence comprises a microRNA or portion thereof. In some embodiments, the nucleic acid sequence comprises an RNA molecule or a fragmented RNA molecule (RNA fragments) selected from: a microRNA (miRNA), a pre-miRNA, a pri-miRNA, a mRNA, a pre-mRNA, a viral RNA, a viroid RNA, a virusoid RNA, circular RNA (circRNA), a ribosomal RNA (IRNA), a transfer RNA (tRNA), a pre-tRNA, a long non-coding RNA (lncRNA), a small nuclear RNA (snRNA), a circulating RNA, a cell-free RNA, an exosomal RNA, a vector-expressed RNA, an RNA transcript, a synthetic RNA, and combinations thereof.

Nucleic acid-based detection techniques that may be useful for the methods herein include quantitative polymerase chain reaction (qPCR), gel electrophoresis, immunochemistry, in situ hybridization such as fluorescent in situ hybridization (FISH), cytochemistry, and next generation sequencing. In some embodiments, the methods involve TaqMan™ qPCR, which involves a nucleic acid amplification reaction with a specific primer pair, and hybridization of the amplified nucleic acids with a hydrolysable probe specific to a target nucleic acid.

In some embodiments, the methods involve hybridization and/or amplification assays that include, but are not limited to, Southern or Northern analyses, polymerase chain reaction analyses, and probe arrays. Non-limiting amplification reactions include, but are not limited to, qPCR, self-sustained sequence replication, transcriptional amplification system, Q-Beta Replicase, rolling circle replication, or any other nucleic acid amplification. As discussed, reference to qPCR herein includes use of TaqMan™ methods. An additional hybridization assay includes the use of nucleic acid probes conjugated or otherwise immobilized on a bead, multi-well plate, or other substrate, wherein the nucleic acid probes are configured to hybridize with a target nucleic acid sequence of a genotype provided herein.

In some embodiments, detecting the presence or absence of a genotype comprises sequencing genetic material from the subject. Sequencing can be performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. Sequencing methods also include next-generation sequencing, e.g., modern sequencing technologies such as Illumina sequencing (e.g., Solexa), Roche 454 sequencing, Ion torrent sequencing, and SOLID sequencing. In some cases, next-generation sequencing involves high-throughput sequencing methods. Additional sequencing methods may also be employed.

In some embodiments, the sequencing the nucleic acid in the sample comprises preparing a sample library for sequencing. In some embodiments, preparing the sample library for sequencing comprises one or more of: sample dilution, cell lysis, tagmentation, tagmentation cleanup, nucleic acid amplification, immobilization, fragmentation, ligation, end repair, barcoding, and combinations hereof.

In some embodiments, a number of nucleotides that are sequenced are at least 5, 10, 15, 20, 25, 30, 35, 40, 45, 50, 100, 150, 200, 300, 400, 500, 2000, 4000, 6000, 8000, 10000, 20000, 50000, 100000, or more than 100000 nucleotides. In some embodiments, the number of nucleotides sequenced is in a range of about 1 to about 100000 nucleotides, about 1 to about 10000 nucleotides, about 1 to about 1000 nucleotides, about 1 to about 500 nucleotides, about 1 to about 300 nucleotides, about 1 to about 200 nucleotides, about 1 to about 100 nucleotides, about 5 to about 100000 nucleotides, about 5 to about 10000 nucleotides, about 5 to about 1000 nucleotides, about 5 to about 500 nucleotides, about 5 to about 300 nucleotides, about 5 to about 200 nucleotides, about 5 to about 100 nucleotides, about 10 to about 100000 nucleotides, about 10 to about 10000 nucleotides, about 10 to about 1000 nucleotides, about 10 to about 500 nucleotides, about 10 to about 300 nucleotides, about 10 to about 200 nucleotides, about 10 to about 100 nucleotides, about 20 to about 100000 nucleotides, about 20 to about 10000 nucleotides, about 20 to about 1000 nucleotides, about 20 to about 500 nucleotides, about 20 to about 300 nucleotides, about 20 to about 200 nucleotides, about 20 to about 100 nucleotides, about 30 to about 100000 nucleotides, about 30 to about 10000 nucleotides, about 30 to about 1000 nucleotides, about 30 to about 500 nucleotides, about 30 to about 300 nucleotides, about 30 to about 200 nucleotides, about 30 to about 100 nucleotides, about 50 to about 100000 nucleotides, about 50 to about 10000 nucleotides, about 50 to about 1000 nucleotides, about 50 to about 500 nucleotides, about 50 to about 300 nucleotides, about 50 to about 200 nucleotides, or about 50 to about 100 nucleotides.

Examples of molecules that are utilized as probes include, but are not limited to, RNA and DNA. In some embodiments, the term “probe” with regards to nucleic acids, refers to any molecule that is capable of selectively binding to a specifically intended target nucleic acid sequence. In some embodiments, probes are specifically designed to be labeled, for example, with a radioactive label, a fluorescent label, an enzyme, a chemiluminescent tag, a colorimetric tag, or other labels or tags. In some embodiments, the fluorescent label comprises a fluorophore. In some embodiments, the fluorophore is an aromatic or heteroaromatic compound. In some embodiments, the fluorophore is a pyrene, anthracene, naphthalene, acridine, stilbene, benzoxazole, indole, benzindole, oxazole, thiazole, benzothiazole, canine, carbocyanine, salicylate, anthranilate, xanthenes dye, coumarin. In some examples, xanthene dyes include, e.g., fluorescein and rhodamine dyes. Fluorescein and rhodamine dyes include, but are not limited to 6-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N; N′-tetramethyl-6-carboxyrhodamine (TAMRA), 6-carboxy-X-rhodamine (ROX). Suitable fluorescent probes also include the naphthylamine dyes that have an amino group in the alpha or beta position. For example, naphthylamino compounds include 1-dimethylaminonaphthyl-5-sulfonate, 1-anilino-8-naphthalene sulfonate and 2-p-toluidinyl-6-naphthalene sulfonate, 5-(2′-aminoethyl)aminonaphthalene-1-sulfonic acid (EDANS). In some examples, coumarins include, e.g., 3-phenyl-7-isocyanatocoumarin; acridines, such as 9-isothiocyanatoacridine and acridine orange; N-(p-(2-benzoxazolyl)phenyl) maleimide; cyanines, such as, e.g., indodicarbocyanine 3 (Cy3), indodicarbocyanine 5 (Cy5), indodicarbocyanine 5.5 (Cy5.5), 3-(-carboxy-pentyl)-3′-ethyl-5,5′-dimethyloxacarbocyanine (CyA); 1H, 5H, 11H, 15H-Xantheno [2,3,4-ij: 5,6,7-i′j′]diquinolizin-18-ium, 9-[2 (or 4)-[[[6-[2,5-dioxo-1-pyrrolidinyl)oxy]-6-oxohexyl]amino]sulfonyl]-4 (or 2)-sulfophenyl]-2,3,6,7,12,13,16,17-octahydro-inner salt (TR or Texas Red); or BODIPY™ dyes. In some cases, the probe comprises FAM as the dye label.

In some embodiments, primers and/or probes described herein for detecting a target nucleic acid are used in an amplification reaction. In some embodiments, the amplification reaction is qPCR. In an example, a qPCR is a method employing a TaqMan™ assay. In some embodiments, qPCR comprises using an intercalating dye. Examples of intercalating dyes include SYBR green I, SYBR green II, SYBR gold, ethidium bromide, methylene blue, Pyronin Y, DAPI, acridine orange, Blue View or phycoerythrin. In some embodiments, the intercalating dye is SYBR.

In some embodiments, a number of amplification cycles for detecting a target nucleic acid in an amplification assay is about 5 to about 30 cycles. In some embodiments, the number of amplification cycles for detecting a target nucleic acid is at least about 5 cycles. In some embodiments, the number of amplification cycles for detecting a target nucleic acid is at most about 30 cycles. In some embodiments, the number of amplification cycles for detecting a target nucleic acid is about 5 to about 10, about 5 to about 15, about 5 to about 20, about 5 to about 25, about 5 to about 30, about 10 to about 15, about 10 to about 20, about 10 to about 25, about 10 to about 30, about 15 to about 20, about 15 to about 25, about 15 to about 30, about 20 to about 25, about 20 to about 30, or about 25 to about 30 cycles.

In one aspect, the methods provided herein for determining the presence, absence, and/or quantity of a nucleic acid sequence comprise an amplification reaction such as qPCR. In one embodiment, genetic material is obtained from a sample of a subject, e.g., a sample of blood or serum. In certain embodiments where nucleic acids are extracted, the nucleic acids are extracted using any technique that does not interfere with subsequent analysis. In certain embodiments, this technique uses alcohol precipitation using ethanol, methanol, or isopropyl alcohol. In certain embodiments, this technique uses phenol, chloroform, or any combination thereof. In certain embodiments, this technique uses cesium chloride. In certain embodiments, this technique uses sodium, potassium or ammonium acetate or any other salt commonly used to precipitate DNA. In certain embodiments, this technique utilizes a column or resin based nucleic acid purification scheme such as those commonly sold commercially, one non-limiting example is the GenElute Bacterial Genomic DNA Kit available from Sigma Aldrich. In certain embodiments, after extraction the nucleic acid is stored in water, Tris buffer, or Tris-EDTA buffer before subsequent analysis. In an embodiment, the nucleic acid material is extracted in water. In some cases, extraction does not comprise nucleic acid purification.

In an example qPCR assay, the nucleic acid sample is combined with primers and probes specific for a target nucleic acid that may or may not be present in the sample, and a DNA polymerase. An amplification reaction is performed with a thermal cycler that heats and cools the sample for nucleic acid amplification, and illuminates the sample at a specific wavelength to excite a fluorophore on the probe and detect the emitted fluorescence. For TaqMan™ methods, the probe may be a hydrolysable probe comprising a fluorophore and quencher that is hydrolyzed by DNA polymerase when hybridized to a target nucleic acid. In some cases, the presence of a target nucleic acid is determined when the number of amplification cycles to reach a threshold value is less than 30, 29, 28, 27, 26, 25, 24, 23, 22, 21, or 20 cycles.

In some embodiments, one target nucleic acid (e.g., a polymorphism) is detected with the methods disclosed herein. In some embodiments, at least 2, 3, 4, 5, 6, 7, 8, 9, or 10 target nucleic acids are detected. In some embodiments, the at least 2, 3, 4, 5, 6, 7, 8, 9, or 10 target nucleic acids are detected in a single multiplexed assay.

To practice the methods and systems provided herein, genetic material may be extracted from a sample obtained from a subject, e.g., a sample of blood or serum. In certain embodiments where nucleic acids are extracted, the nucleic acids are extracted using any technique that does not interfere with subsequent analysis. In certain embodiments, this technique uses alcohol precipitation using ethanol, methanol or isopropyl alcohol. In certain embodiments, this technique uses phenol, chloroform, or any combination thereof. In certain embodiments, this technique uses cesium chloride. In certain embodiments, this technique uses sodium, potassium or ammonium acetate or any other salt commonly used to precipitate DNA. In certain embodiments, this technique utilizes a column or resin based nucleic acid purification scheme such as those commonly sold commercially, one non-limiting example is the GenElute Bacterial Genomic DNA Kit available from Sigma Aldrich. In certain embodiments, after extraction the nucleic acid is stored in water, Tris buffer, or Tris-EDTA buffer before subsequent analysis. In an example embodiment, the nucleic acid material is extracted in water. In some cases, extraction does not comprise nucleic acid purification. In certain embodiments, RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland).

In some embodiments, the sample is assayed to measure a presence, absence or quantity of one polymorphisms. In some embodiments, the sample is assayed to measure a presence, absence or quantity of at least two polymorphisms. In some embodiments, the sample is assayed to measure a presence, absence or quantity of at least three polymorphisms. In some embodiments, the sample is assayed to measure a presence, absence, or quantity of at least four polymorphisms. In some embodiments, the sample is assayed to measure a presence, absence, or quantity of at least five polymorphisms.

### Methods of Detecting a Marker

In some embodiments, methods of detecting a presence, absence, or level of a target protein (e.g., biomarker) in the sample obtained from the subject involve detecting protein activity or expression. In some embodiments, the target protein is ANCA or ASCA. A target protein may be detected by use of an antibody-based assay, where an antibody specific to the target protein is utilized. In some embodiments, antibody-based detection methods utilize an antibody that binds to any region of target protein. In one example, the method of analysis comprises performing an enzyme-linked immunosorbent assay (ELISA). The ELISA assay may be a sandwich ELISA or a direct ELISA. Another example method of analysis comprises a single molecule array, e.g., Simoa. Other examples of methods of detection include immunohistochemistry and lateral flow assay. Other examples of methods for detecting target protein include, but are not limited to, gel electrophoresis, capillary electrophoresis, high performance liquid chromatography (HPLC), thin layer chromatography (TLC), hyperdiffusion chromatography, and the like, or various immunological methods such as fluid or gel precipitation reactions, immunodiffusion (single or double), immunoelectrophoresis, radioimmunoassay (RIA), immunofluorescent assays, and Western blotting. In some embodiments, antibodies, or antibody fragments, are used in methods such as Western blots or immunofluorescence techniques to detect the expressed proteins. The antibody or protein can be immobilized on a solid support for Western blots and immunofluorescence techniques. Suitable solid phase supports or carriers include any support capable of binding an antigen or an antibody. In some embodiments, supports or carriers include glass, polystyrene, polypropylene, polyethylene, dextran, nylon, amylases, natural and modified celluloses, polyacrylamides, gabbros, and magnetite.

In some cases, a target protein may be detected by detecting binding between the target protein and a binding partner of the target protein. In some embodiments, methods of analysis of protein-protein binding comprise performing an assay in vivo or in vitro, or ex vivo. In some embodiments, the method of analysis comprises an assay such as a co-immunoprecipitation (co-IP), pull-down, crosslinking protein interaction analysis, labeled transfer protein interaction analysis, or Far-western blot analysis, FRET based assay, including, for example FRET-FLIM, a yeast two-hybrid assay, BiFC, or split luciferase assay.

Disclosed herein are methods of detecting a presence or a level of one or more serological markers in a sample obtained from a subject. In some embodiments, the one or more serological markers comprises anti-Saccharomyces cerevisiae antibody (ASCA), an anti-neutrophil cytoplasmic antibody (ANCA), antibody against E. coli outer membrane porin protein C (anti-OmpC), anti-chitin antibody, pANCA antibody, anti-12 antibody, and anti-Cbirl flagellin antibody. In some embodiments, the antibodies comprises immunoglobulin A (IgA), immunoglobulin G (IgG), immunoglobulin E (IgE), or immunoglobulin M (IgM), immunoglobulin D (IgD), or a combination thereof. Any suitable method for detecting a target protein or biomarker disclosed herein may be used to detect a presence, absence, or level of a serological marker. In some embodiments, the presence or the level of the one or more serological markers is detected using an enzyme-linked immunosorbent assay (ELISA), a single molecule array (Simoa), immunohistochemistry, internal transcribed spacer (ITS) sequencing, or any combination thereof. In some embodiments, the ELISA is a fixed leukocyte ELISA. In some embodiments, the ELISA is a fixed neutrophil ELISA. A fixed leukocyte or neutrophil ELISA may be useful for the detection of certain serological markers, such as those described in Saxon et al., A distinct subset of antineutrophil cytoplasmic antibodies is associated with inflammatory bowel disease, J. Allergy Clin. Immuno. 86:2; 202-210 (August 1990). In some embodiments, ELISA units (EU) are used to measure positivity of a presence or level of a serological marker (e.g., seropositivity), which reflects a percentage of a standard or reference value. In some embodiments, the standard comprises pooled sera obtained from well-characterized patient population (e.g., diagnosed with the same disease or condition the subject has, or is suspected of having) reported as being seropositive for the serological marker of interest. In some embodiments, measuring seropositivity or seronegativity comprises calculating a percentage of a reference value from pooled sera obtained from a patient population diagnosed with CD. In some embodiments, the control or reference value comprises 10, 20, 30, 40, 50, 60, 70, 80, 90, or 100 EU. In some embodiments, a quartile sum scores are calculated using, for example, the methods reported in Landers C J, Cohavy O, Misra R. et al., Selected loss of tolerance evidenced by Crohn's disease-associated immune responses to auto- and microbial antigens. Gastroenterology (2002) 123:689-699.

### Methods of Treatment

In some embodiments of the methods described herein, a subject is administered a therapeutic agent. In some embodiments, a therapeutically effective amount of the therapeutic agent described herein is administered, wherein one or more POPP markers were detected in the subject. In some embodiments, the therapeutic agent is determined based, at least in part on the one or more POPP markers detected in the subject. In some embodiments, the subject is receiving a therapeutic agent for the treatment of CD.

In some embodiments the therapeutic agent comprises an inhibitor of tumor necrosis factor alpha (TNFα), an inhibitor of IL23, an inhibitor of integrin, an inhibitor of SIPR, an inhibitor of TNF Superfamily Member 15 (TL1A), or an inhibitor of JAK, or any combination thereof.

### TNF Inhibitors

In some embodiments, the TNF inhibitors for the combination therapy comprise any one selected from the group consisting of adalimumab, infliximab, golimumab, certolizumab, and etanercept. In certain embodiments, the TNF inhibitors are anti-TNF antibodies or antigen-binding fragments. In some embodiments, the TNF inhibitors comprise a TNF receptor. In some embodiments, the TNF inhibitors comprise a TNF receptor fusion protein, e.g., a TNF receptor fused to an antibody Fc region. In some embodiments, the TNF inhibitors consist of anti-TNF antibodies or antigen-binding fragments thereof, or TNF receptor comprising proteins. In some embodiments, the TNF inhibitors for the combination therapy comprise any one selected from the group consisting of adalimumab, infliximab, golimumab, certolizumab, and etanercept. In certain embodiments, the TNF inhibitors comprise any one selected from the group consisting of variants of adalimumab, variants of infliximab, variants of golimumab, variants of certolizumab, and variants of etanercept.

In some embodiments, the TNF inhibitor provided herein for the combination therapy comprises adalimumab. In certain embodiments, the TNF inhibitor comprises an anti-TNF antibody or antigen binding fragment selected from those described in U.S. Pat. Nos. 6,805,686; 8,231,876; 8,420,081; 8,663,945; 8,708,968; 8,715,664; 8,808,700; 8,883,156; 8,889,136; 8,895,009; 8,906,372; 8,906,373; 8,906,646; 8,911,737; 8,911,964; 8,916,153; 8,926,975; 8,961,973; 8,961,974; 8,974,790; 8,986,693; 8,992,926; 8,999,337; 9,061,005; 9,062,106; 9,067,992; 9,085,618; 9,085,619; 9,085,620; 9,090,688; 9,090,689; 9,090,867; 9,096,666; 9,102,723; 9,150,645; 9,181,337; 9,181,572; 9,187,559; 9,234,032; 9,266,949; 9,273,132; 9,284,370; 9,284,371; 9,290,568; 9,315,574; 9,328,165; 9,334,319; 9,339,610; 9,346,879; 9,359,434; 9,499,614; 9,499,616; 9,505,834; 9,512,216; 9,522,953; 9,546,212; 9,550,826; 9,624,295; 9,669,093; 9,683,033; 9,708,400; 9,913,902; 9,957,318; 11,083,792; and 11,167,030, the disclosure of all of which are hereby incorporated in their entireties by reference. In one embodiment, the TNF inhibitor comprises an anti-TNF antibody or antigen binding fragment at a formulation and dose as described in U.S. Pat. Nos. 6,805,686; 8,231,876; 8,420,081; 8,663,945; 8,708,968; 8,715,664; 8,808,700; 8,883,156; 8,889,136; 8,895,009; 8,906,372; 8,906,373; 8,906,646; 8,911,737; 8,911,964; 8,916,153; 8,926,975; 8,961,973; 8,961,974; 8,974,790; 8,986,693; 8,992,926; 8,999,337; 9,061,005; 9,062,106; 9,067,992; 9,085,618; 9,085,619; 9,085,620; 9,090,688; 9,090,689; 9,090,867; 9,096,666; 9,102,723; 9,150,645; 9,181,337; 9,181,572; 9,187,559; 9,234,032; 9,266,949; 9,273,132; 9,284,370; 9,284,371; 9,290,568; 9,315,574; 9,328,165; 9,334,319; 9,339,610; 9,346,879; 9,359,434; 9,499,614; 9,499,616; 9,505,834; 9,512,216; 9,522,953; 9,546,212; 9,550,826; 9,624,295; 9,669,093; 9,683,033; 9,708,400; 9,913,902; 9,957,318; 11,083,792; and 11,167,030, the disclosures of all of which are hereby incorporated in their entireties by reference.

In certain embodiments, the TNF inhibitor comprises an anti-TNF antibody or antigen binding fragment as described in the US FDA approved label for HUMIRA (revised February 2021, available at https://www.accessdata.fda.gov/drugsatfda_docs/label/2021/125057s4171bl.pdf). In one embodiment, the anti-TNF comprises an anti-TNF antibody or antigen binding fragment as described in the US FDA approved label for HUMIRA (revised February 2021, available at https://www.accessdata.fda.gov/drugsatfda_docs/label/2021/125057s4171bl.pdf), at a formulation and dose as described in the same US FDA approved label, the disclosures of all of which are hereby incorporated in their entireties by reference.

In some embodiments, the TNF inhibitor provided herein for the combination therapy comprises infliximab. In certain embodiments, the TNF inhibitor comprises an anti-TNF antibody or antigen-binding fragment thereof selected from those described in U.S. Pat. Nos. 7,754,853, 8,722,855, 9,580,501, 6,790,444, 6,835,823, 6,991,791, 7,101,674, 7,135,178, 7,135,179, 7,128,907, 7,128,908, 7,160,543, 7,160,995, 7,169,386, 7,169,388, 7,166,284, 7,179,466, 7,204,985, 7,214,376, 7,223,396, 7,226,593, 7,227,003, 7,335,358, 11,149,085, 5,698,195, 5,919,452, 6,277,969, and 6,284,471, the disclosures of all of which are hereby incorporated in their entireties by reference. In one embodiment, the TNF inhibitor comprises an anti-TNF antibody or antigen binding fragment at a formulation and dose as described in U.S. Pat. Nos. 7,754,853, 8,722,855, 9,580,501, 6,790,444, 6,835,823, 6,991,791, 7,101,674, 7,135,178, 7,135,179, 7,128,907, 7,128,908, 7,160,543, 7,160,995, 7,169,386, 7,169,388, 7,166,284, 7,179,466, 7,204,985, 7,214,376, 7,223,396, 7,226,593, 7,227,003, 7,335,358, 11,149,085, 5,698,195, 5,919,452, 6,277,969, and 6,284,471, the disclosures of all of which are hereby incorporated in their entireties by reference.

In certain embodiments, the TNF inhibitor comprises an anti-TNF antibody or antigen-binding fragment thereof as described in the US FDA approved label for Remicade (revised October 2021, available at https://www.accessdata.fda.gov/drugsatfda_docs/label/2021/103772s54011bl.pdf). In one embodiment, the TNF inhibitor comprises an anti-TNF antibody or antigen-binding fragment thereof as described in the US FDA approved label for Remicade (revised October 2021, available at https://www.accessdata.fda.gov/drugsatfda_docs/label/2021/103772s54011bl.pdf), at a formulation and dose as described in the same US FDA approved label, the disclosures of all of which are hereby incorporated in their entireties by reference.

In some embodiments, the TNF inhibitor provided herein for the combination therapy comprises golimumab. In certain embodiments, the TNF inhibitor comprises an anti-TNF antibody or antigen binding fragment selected from those described in U.S. Pat. Nos. 7,250,165; 7,691,378; 7,521,206; 7,815,909; 7,820,169; 8,241,899; 8,603,778; 9,321,836; 9,828,424; 10,465,003; 11,014,982; and 11,041,020; 9,072,668; 10,059,763; 10,793,629; 8,722,855; 10,465,003; 11,014,982; 11,041,020; and 11,053,303, the disclosures of all of which are hereby incorporated in their entireties by reference. In one embodiment, the TNF inhibitor comprises an anti-TNF antibody or antigen binding fragment at a formulation and dose as described in U.S. Pat. Nos. 7,250,165; 7,691,378; 7,521,206; 7,815,909; 7,820,169; 8,241,899; 8,603,778; 9,321,836; 9,828,424; 10,465,003; 11,014,982; 11,041,020; 9,072,668; 10,059,763; 10,793,629; 8,722,855; 10,465,003; 11,014,982; 11,041,020; and 11,053,303, the disclosures of all of which are hereby incorporated in their entireties by reference.

In certain embodiments, the TNF inhibitor comprises an anti-TNF antibody or antigen binding fragment as described in the US FDA approved label for SIMPONI (revised September 2019, available at https://www.accessdata.fda.gov/drugsatfda_docs/label/2019/125289s1461bl.pdf). In one embodiment, the TNF inhibitor comprises an anti-TNF antibody or antigen binding fragment as described in the US FDA approved label for SIMPONI (revised September 2019, available at https://www.accessdata.fda.gov/drugsatfda_docs/label/2019/125289s1461bl.pdf), at a formulation and dose as described in the same US FDA approved label, the disclosures of all of which are hereby incorporated in their entireties by reference.

In some embodiments, the TNF inhibitor provided herein for the combination therapy comprises certolizumab. In certain embodiments, the TNF inhibitor comprises an anti-TNF antibody or antigen binding fragment selected from those described in U.S. Pat. Nos. 7,402,662, 8,062,865, 8,722,353, 9,096,887, 9,751,930, 11,053,303, and 11,104,745, and EP U.S. Pat. Nos. 3,165,928 and 2,791,172., the disclosures of all of which are hereby incorporated in their entireties by reference. In one embodiment, the TNF inhibitor comprises an anti-TNF antibody or antigen binding fragment at a formulation and dose as described in U.S. Pat. Nos. 7,402,662, 8,062,865, 8,722,353, 9,096,887, 9,751,930, 11,053,303, and 11,104,745, the disclosures of all of which are hereby incorporated in their entireties by reference.

In certain embodiments, the TNF inhibitor comprises an anti-TNF antibody or antigen binding fragment as described in the US FDA approved label for CIMZIA (revised September 2019, available at https://www.accessdata.fda.gov/drugsatfda_docs/label/2019/125160s2931bl.pdf). In one embodiment, the TNF inhibitor comprises an anti-TNF antibody or antigen binding fragment as described in the US FDA approved label for CIMZIA (revised September 2019, available at https://www.accessdata.fda.gov/drugsatfda_docs/label/2019/125160s2931bl.pdf), at a formulation and dose as described in the same US FDA approved label, the disclosures of all of which are hereby incorporated in their entireties by reference.

In some embodiments, the TNF inhibitor provided herein for the combination therapy comprises etanercept. In certain embodiments, the TNF inhibitor is selected from TNF inhibitors as described in U.S. Pat. Nos. 5,712,155, RE36755, 8163522, 5605690, 8063182, 9884911, 10457728, 11053307, 10000551, 10087232, 10730925, 9182410, 9598718, 10307483, 11129876, 11000588, 10954293, and 7276477, and US Patent Publication Nos. 2021/0007991 and 2006/0009450, the disclosures of all of which are hereby incorporated in their entireties by reference. In one embodiment, the TNF inhibitor comprises a TNF inhibitor at a formulation and dose as described in U.S. Pat. Nos. 5,712,155, RE36755, 8163522, 5605690, 8063182, 9884911, 10457728, 11053307, 10000551, 10087232, 10730925, 9182410, 9598718, 10307483, 11129876, 11000588, 10954293, and 7276477, and US Patent Publication Nos. 2021/0007991 and 2006/0009450, the disclosures of all of which are hereby incorporated in their entireties by reference.

In certain embodiments, the TNF inhibitor is as described in the US FDA approved label for ENBREL (revised February 2021, available at https://www.accessdata.fda.gov/drugsatfda_docs/label/2021/103795s55821bl.pdf). In one embodiment, the TNF inhibitor is as described in the US FDA approved label for ENBREL (revised February 2021, available at https://www.accessdata.fda.gov/drugsatfda_docs/label/2021/103795s55821bl.pdf), at a formulation and dose as described in the same US FDA approved label, the disclosures of all of which are hereby incorporated in their entireties by reference.

In some embodiments, the TNF inhibitor provided herein for the combination therapy comprises anti-TNF antibodies or antigen-binding fragments thereof. In certain embodiments, the TNF inhibitor provided herein for the combination therapy comprises anti-TNF antibodies or antigen-binding fragments thereof that are specific for a TNF, such as TNF alpha. In one embodiment, the TNF inhibitor provided herein for the combination therapy comprises anti-TNF antibodies or antigen-binding fragments thereof, wherein the anti-TNF antibodies or antigen-binding fragments thereof are antagonist of TNFs. In one embodiment, the TNF inhibitor provided herein for the combination therapy comprises anti-TNF antibodies or antigen-binding fragments thereof, wherein the anti-TNF antibodies or antigen-binding fragments thereof inhibits binding to TNF.

### IL23 Inhibitors

In some embodiments, the IL23 inhibitors comprise anti-IL23 antibodies or antigen-binding fragments. In certain embodiments, the IL23 inhibitors are anti-IL23 antibodies or antigen-binding fragments. In some embodiments, the IL23 inhibitors consists of anti-IL23 antibodies or antigen-binding fragments. In some embodiments, the IL23 inhibitors comprise any one selected from the group consisting of ustekinumab, guselkumab, risankizumab, brazikumab, mirikizumab, tildrakizumab, and briakinumab. In certain embodiments, the IL23 inhibitors comprise any one selected from the group consisting of variants of ustekinumab, variants of guselkumab, variants of risankizumab, variants of brazikumab, variants of mirikizumab, variants of tildrakizumab, and variants of briakinumab.

In some embodiments, the IL23 inhibitor provided herein comprises ustekinumab. In certain embodiments, the IL23 inhibitor comprises an anti-IL23 antibody or antigen binding fragment selected from those described in U.S. Pat. Nos. 6,902,734, 7,887,807, 8,703,141, 9,676,848, 11078267, 10765724, and 11197913, wherein the anti-IL23 antibody or antigen binding fragment also binds to IL12. In one embodiment, the IL23 inhibitor comprises an anti-IL23 antibody or antigen binding fragment selected from those described in U.S. Pat. Nos. 6,902,734, 7,887,807, 8,703,141, 9,676,848, 11,078,267, 10,765,724, and 11,197,913, at a formulation and dose as described in U.S. Pat. Nos. 6,902,734, 7,887,807, 8,703,141, 9,676,848, 11,078,267, 10,765,724, and 11,197,913, the disclosures of all of which are hereby incorporated in their entireties by reference.

In certain embodiments, the IL23 inhibitor comprises an anti-IL23 antibody or antigen binding fragment as described in the US FDA approved label for STELARA (revised December 2020, available at accessdata.fda.gov/drugsatfda_docs/label/2020/125261s154,761044s0061bl.pdf). In one embodiment, the IL23 inhibitor comprises an anti-IL23 antibody or antigen binding fragment as described in the US FDA approved label for STELARA (revised December 2020, available at accessdata.fda.gov/drugsatfda_docs/label/2020/125261s154,761044s0061bl.pdf), at a formulation and dose as described in the same US FDA approved label, the disclosures of all of which are hereby incorporated in their entireties by reference.

In some embodiments, the IL23 inhibitor provided herein comprises guselkumab. In certain embodiments, the IL23 inhibitor comprises guselkumab or a variant of guselkumab selected from those described in U.S. Pat. Nos. 7,935,344, 7,993,645, 8,221,760, 9,783,607, 10,954,297, and 11,208,474. In one embodiment, the IL23 inhibitor comprises guselkumab or a variant of guselkumab selected from those described in U.S. Pat. Nos. 7,935,344, 7,993,645, 8,221,760, 9,783,607, 10,954,297, and 11,208,474, at a formulation and dose as described in U.S. Pat. Nos. 7,935,344, 7,993,645, 8,221,760, 9,783,607, 10,954,297, and 11,208,474, the disclosures of all of which are hereby incorporated in their entireties by reference.

In certain embodiments, the IL23 inhibitor comprises guselkumab as described in the US FDA approved label for TREMFYA (revised July 2020, available at accessdata.fda.gov/drugsatfda_docs/label/2020/761061s0071bl.pdf). In one embodiment, the IL23 inhibitor comprises guselkumab as described in the US FDA approved label for TREMFYA (revised July 2020, available at accessdata.fda.gov/drugsatfda_docs/label/2020/761061s0071bl.pdf), at a formulation and dose as described in the same US FDA approved label, the disclosures of all of which are hereby incorporated in their entireties by reference.

In some embodiments, the IL23 inhibitor provided herein comprises Risankizumab. In certain embodiments, the IL23 inhibitor comprises an anti-IL23 antibody or antigen-binding fragment selected from those described in U.S. Pat. Nos. 8,778,346, 9,441,036, and 10,202,448. In one embodiment, the IL23 inhibitor comprises an anti-IL23 antibody or antigen-binding fragment selected from those described in U.S. Pat. Nos. 8,778,346, 9,441,036, and 10,202,448, at a formulation and dose as described in U.S. Pat. Nos. 8,778,346, 9,441,036, and 10,202,448, the disclosures of all of which are hereby incorporated in their entireties by reference.

In certain embodiments, the IL23 inhibitor comprises risankizumab as described in the US FDA approved label for SKYRIZI (revised April 2021, available at accessdata.fda.gov/scripts/cder/daf/index.cfm?event-overview.process&ApplNo=761105). In one embodiment, the IL23 inhibitor comprises risankizumab as described in the US FDA approved label for SKYRIZI (revised April 2021, available at accessdata.fda.gov/scripts/cder/daf/index.cfm?event-overview.process&ApplNo=761105), at a formulation and dose as described in the same US FDA approved label, the disclosures of all of which are hereby incorporated in their entireties by reference.

In some embodiments, the IL23 inhibitor provided herein comprises brazikumab. In certain embodiments, the IL23 inhibitor comprises an anti-IL23 antibody or antigen binding fragment selected from those described in U.S. Pat. Nos. 8,722,033, 9,487,580, and 9,951,129 and US Publication Nos. US20210277105A1 (U.S. Ser. No. 17/259,448) and US20210079086A1 (U.S. Ser. No. 16/999,470). In one embodiment, the IL23 inhibitor comprises an ozanimod or a derivative of ozanimod selected from those described in U.S. Pat. Nos. 8,722,033, 9,487,580, and 9,951,129 and US Publication Nos. US20210277105A1 (U.S. Ser. No. 17/259,448) and US20210079086A1 (U.S. Ser. No. 16/999,470), at a formulation and dose as described in U.S. Pat. Nos. 8,722,033, 9,487,580, and 9,951,129 and US Publication Nos. US20210277105A1 (U.S. Ser. No. 17/259,448) and US20210079086A1 (U.S. Ser. No. 16/999,470), the disclosures of all of which are hereby incorporated in their entireties by reference.

In some embodiments, the IL23 inhibitor provided herein comprises mirikizumab. In certain embodiments, the IL23 inhibitor comprises an anti-IL23 antibody or antigen binding fragment selected from those described in U.S. Pat. Nos. 9,023,358 and 9,688,753, and in the publication of WO/2020/219314 (PCT/US2020/028273). In one embodiment, the IL23 inhibitor comprises an anti-IL23 antibody or antigen binding fragment selected from those described in U.S. Pat. Nos. 9,023,358 and 9,688,753, and in the publication of WO/2020/219314 (PCT/US2020/028273), at a formulation and dose as described in U.S. Pat. Nos. 9,023,358 and 9,688,753, and in the publication of WO/2020/219314 (PCT/US2020/028273), the disclosures of all of which are hereby incorporated in their entireties by reference.

In some embodiments, the IL23 inhibitor provided herein comprises tildrakizumab. In certain embodiments, the IL23 inhibitor comprises an anti-IL23 antibody or antigen binding fragment thereof selected from those described in U.S. Pat. Nos. 8,404,813, 8,293,883, and 9,809,648. In one embodiment, the IL23 inhibitor comprises an amiselimod or a derivative of amiselimod selected from those described in U.S. Pat. Nos. 8,404,813, 8,293,883, and 9,809,648, at a formulation and dose as described in U.S. Pat. Nos. 8,404,813, 8,293,883, and 9,809,648, the disclosures of all of which are hereby incorporated in their entireties by reference.

In certain embodiments, the IL23 inhibitor comprises tildrakizumab as described in the US FDA approved label for ILUMYA (revised March 2018, available at accessdata.fda.gov/drugsatfda_docs/label/2018/761067s0001bl.pdf). In one embodiment, the IL23 inhibitor comprises tildrakizumab as described in the US FDA approved label for ILUMYA (revised March 2018, available at accessdata.fda.gov/drugsatfda_docs/label/2018/761067s0001bl.pdf), at a formulation and dose as described in the same US FDA approved label, the disclosures of all of which are hereby incorporated in their entireties by reference.

In some embodiments, the IL23 inhibitor provided herein comprises briakinumab. In certain embodiments, the IL23 inhibitor comprises an anti-IL23 antibody or antigen binding fragment thereof selected from those described in U.S. Pat. Nos. 6,914,128, 7,504,485, 8,865,174, 9,035,030, and 8,557,239. In one embodiment, the IL23 inhibitor comprises an anti-IL23 antibody or antigen binding fragment thereof selected from those described in U.S. Pat. Nos. 6,914,128, 7,504,485, 8,865,174, 9,035,030, and 8,557,239, at a formulation and dose as described in U.S. Pat. Nos. 6,914,128, 7,504,485, 8,865,174, 9,035,030, and 8,557,239, the disclosures of all of which are hereby incorporated in their entireties by reference.

In certain embodiments, the IL23 inhibitor comprises an anti-IL23 antibody or antigen binding fragment thereof as described in Grodon K. B. et al., J Invest Dermatol. 2012 February; 132 (2): 304-14, and Remo Panaccione et al., Inflamm Bowel Dis. 2015 June; 21 (6): 1329-40. In one embodiment, the IL23 inhibitor comprises an anti-IL23 antibody or antigen binding fragment thereof as described in Grodon K. B. et al., J Invest Dermatol. 2012 February; 132 (2): 304-14, and Remo Panaccione et al., Inflamm Bowel Dis. 2015 June; 21 (6): 1329-40, at a formulation and dose as described in the same publications, the disclosures of all of which are hereby incorporated in their entireties by reference.

### Integrin Inhibitors

In some embodiments, the integrin inhibitors comprise any one selected from the group consisting of etrolizumab, vedolizumab, natalizumab, and ontamalimab. In certain embodiments, the integrin inhibitors are anti-integrin antibodies or antigen-binding fragments. In some embodiments, the integrin inhibitors consist of anti-integrin antibodies or antigen-binding fragments thereof. In certain embodiments, the integrin inhibitors comprise any one selected from the group consisting of variants of etrolizumab, variants of vedolizumab, variants of natalizumab, and variants of ontamalimab.

In some embodiments, the integrin inhibitor provided comprises etrolizumab. In certain embodiments, the integrin inhibitor comprises an anti-integrin antibody or antigen binding fragment selected from those described in U.S. Pat. Nos. 9,873,742, 10,273,542, 10,669,587, and/or 11,091,551, or US Patent Application Nos. 2020/0392232 and 2020/0048354. In one embodiment, the integrin inhibitor comprises an anti-integrin antibody or antigen binding fragment selected from those described in U.S. Pat. Nos. 9,873,742, 10,273,542, 10,669,587, and/or 11,091,551, or US Patent Application Nos. 2020/0392232 and 2020/0048354, at a formulation and dose as described in U.S. Pat. Nos. 9,873,742, 10,273,542, 10,669,587, and/or 11,091,551, or US Patent Application Nos. 2020/0392232 and 2020/0048354, the disclosures of all of which are hereby incorporated in their entireties by reference.

In some embodiments, the integrin inhibitor provided herein comprises vedolizumab. In certain embodiments, the integrin inhibitor comprises an anti-integrin antibody or antigen binding fragment selected from those described in U.S. Pat. Nos. 7,147,851, 10,004,808, 10,040,855, 10,143,752, 10,232,056, 10,239,942, 10,513,561, 10,570,196, 10,676,525, 10,858,442, 10,864,268, 10,918,716, 11,014,985, 11,084,872, 7,402,410, 8,021,661, 8,333,967, 8,937,163, 9,017,675, 9,181,332, 9,371,389, 9,382,320, 9,388,222, 9,393,302, 9,499,633, 9,663,579, 9,764,033, 9,828,426, US Patent Application No. 2020/0031937, and AU Patent Application No. 2019/216679, at a formulation and dose as described in U.S. Pat. Nos. 7,147,851, 10,004,808, 10,040,855, 10,143,752, 10,232,056, 10,239,942, 10,513,561, 10,570,196, 10,676,525, 10,858,442, 10,864,268, 10,918,716, 11,014,985, 11,084,872, 7,402,410, 8,021,661, 8,333,967, 8,937,163, 9,017,675, 9,181,332, 9,371,389, 9,382,320, 9,388,222, 9,393,302, 9,499,633, 9,663,579, 9,764,033, 9,828,426, US Patent Application No. 2020/0031937, and AU Patent Application No. 2019/216679, the disclosures of all of which are hereby incorporated in their entireties by reference.

In certain embodiments, the integrin inhibitor comprises an anti-integrin antibody or antigen binding fragment as described in the US FDA approved label for ENTYVIO (updated August 2021, https://www.accessdata.fda.gov/drugsatfda_docs/label/2021/125476s038s0391bl.pdf). In one embodiment, the integrin inhibitor comprises an anti-integrin antibody or antigen binding fragment as described in the US FDA approved label for ENTYVIO (updated August 2021, https://www.accessdata.fda.gov/drugsatfda_docs/label/2021/125476s038s0391bl.pdf), at a formulation and dose as described in the same US FDA approved label, the disclosures of all of which are hereby incorporated in its entirety by reference.

In some embodiments, the integrin inhibitor provided herein comprises natalizumab. In certain embodiments, the integrin inhibitor comprises an anti-integrin antibody or antigen-binding fragment thereof selected from those described in U.S. Pat. Nos. 10,466,251, 9,533,044, 6,033,665, 5,840,299, US Patent Publication Nos. 2004/009169 and 2021/0322549, DK Patent Publication No. 1734997, and KR Patent Publication No. 2020/0088350. In one embodiment, the anti-integrin comprises an anti-integrin antibody or antigen-binding fragment thereof selected from those described in U.S. Pat. Nos. 10,466,251, 9,533,044, 6,033,665, and 5,840,299, US Patent Publication Nos. 2004/009169 and 2021/0322549, DK Patent Publication No. 1734997, and KR Patent Publication No. 2020/0088350, at a formulation and dose as described in U.S. Pat. Nos. 6,033,665, 9,533,044, 10,466,251 and 5,840,299, US Patent Publication Nos. 2004/009169 and 2021/0322549, DK Patent Publication No. 1734997, and KR Patent Publication No. 2020/0088350, the disclosures of all of which are hereby incorporated in their entireties by reference.

In certain embodiments, the integrin inhibitor comprises an anti-integrin antibody or antigen-binding fragment thereof as described in the US FDA approved label for TYSABRI (revised August 2021, available at https://www.accessdata.fda.gov/scripts/cder/daf/index.cfm?event-overview.process&ApplNo=1 25476). In one embodiment, the anti-integrin comprises an anti-integrin antibody or antigen-binding fragment thereof as described in the US FDA approved label for TYSABRI (revised August 2021, available at https://www.accessdata.fda.gov/scripts/cder/daf/index.cfm?event-overview.process&ApplNo=1 25476), at a formulation and dose as described in the same US FDA approved label, the disclosures of all of which are hereby incorporated in their entireties by reference.

In some embodiments, the integrin inhibitor provided herein comprises ontamalimab. In certain embodiments, the integrin inhibitor comprises an anti-integrin antibody or antigen binding fragment selected from those described in U.S. Pat. Nos. 9,328,169, 10,851,163, 7,932,372, or 7,935,794, at a formulation and dose as described in U.S. Pat. Nos. 9,328,169, 10,851,163, 7,932,372, or 7,935,794, the disclosures of which are hereby incorporated in its entirety by reference.

### S1PR Inhibitors

Similarly, SIPR inhibitors provided herein can have various functional properties and be of various modalities. In some embodiments, S1PR inhibitors comprise molecules that can block, inhibit, attenuate, or reduce SIPR-mediated or SIPR-dependent signaling in a cell expressing an SIPR. In some embodiments, SIPR inhibitors comprise molecules that can block, inhibit, attenuate, or reduce SIPR expression in the cells. In some embodiments, SIPR inhibitors comprise molecules that can block, inhibit, attenuate, or reduce SIPR protein levels in the cells. In some embodiments, S1PR inhibitors comprise molecules that can block, inhibit, attenuate, or reduce S1PR binding to a natural S1PR ligand such as sphingosine-1-phosphate (SIP). In certain embodiments, S1PR inhibitors comprise small molecule S1PR inhibitors. In certain embodiments, S1PR inhibitors comprise functional antagonists. In certain embodiments, S1PR inhibitors comprise small molecule functional antagonists. In certain embodiments, SIPR inhibitors comprise anti-SIPR antibodies. In certain embodiments, SIPR inhibitors comprise any S1PR inhibitor described herein. In certain embodiments, S1PR inhibitors comprise inhibitory anti-S1PR antibodies or antigen binding fragments thereof. In certain embodiments, S1PR inhibitors comprise siRNA molecules against S1PR mRNA.

In some embodiments, the SIPR inhibitors comprise any one selected from the group consisting of fingolimod, siponimod, etrasimod, ozanimod, ponesimod, amiselimod, ceralifimod, and mocravimod. In certain embodiments, the SIPR inhibitors comprise any one selected from the group consisting of derivatives of fingolimod, derivatives of siponimod, derivatives of etrasimod, derivatives of ozanimod, derivatives of ponesimod, derivatives of amiselimod, derivatives of ceralifimod, and derivatives of mocravimod. Derivatives when used in reference to a compound (such as the SIPR inhibitors of this paragraph) means a chemically modified compound wherein the modulation is considered routine by the ordinary skilled chemist such as additional chemical moieties (e.g., an ester or an amide of an acid, protecting groups, such as benzyl group for an alcohol or thiol, and ter-butosycarbonyl group for an amine).

As described further above, in some embodiments, the SIPR inhibitor provided herein is both an agonist of S1PR and is a functional antagonist of S1PR.

In certain embodiments, the S1PR inhibitor comprises a fingolimod or a derivative of fingolimod selected from those described in U.S. Pat. Nos. 9,592,208, 9,187,405, 10,543,179, 6,277,888, and 8,324,283. In one embodiment, the S1PR inhibitor comprises a fingolimod or a derivative of fingolimod selected from those described in U.S. Pat. Nos. 9,592,208, 9,187,405, 10,543,179, 6,277,888, and 8,324,283, at a formulation and dose as described in U.S. Pat. Nos. 9,592,208, 9,187,405, 10,543,179, 6,277,888, and 8,324,283, the disclosures of all of which are hereby incorporated in their entireties by reference.

In certain embodiments, the SIPR inhibitor comprises a siponimod or a derivative of siponimod selected from those described in U.S. Pat. Nos. 8,492,441 and 7,939,519. In one embodiment, the S1PR inhibitor comprises a siponimod or a derivative of siponimod selected from those described in U.S. Pat. Nos. 8,492,441 and 7,939,519, at a formulation and dose as described in U.S. Pat. Nos. 8,492,441 and 7,939,519, the disclosures of all of which are hereby incorporated in their entireties by reference.

In certain embodiments, the S1PR inhibitor comprises an etrasimod or a derivative of etrasimod selected from those described in U.S. Pat. Nos. 11,007,175, 10,676,435, and 10,301,262, and US Publication Nos. US20210338636A1, US20210228545A1, US20200016121A1. In one embodiment, the S1PR inhibitor comprises an etrasimod or a derivative of etrasimod selected from those described in U.S. Pat. Nos. 11,007,175, 10,676,435, and 10,301,262, and US Publication Nos. US20210338636A1, US20210228545A1, US20200016121A1, at a formulation and dose as described in U.S. Pat. Nos. 11,007,175, 10,676,435, and 10,301,262, and US Publication Nos. US20210338636A1, US20210228545A1, US20200016121A1, the disclosures of all of which are hereby incorporated in their entireties by reference. In some embodiments, the S1PR inhibitor or any crystalline form thereof, can be prepared using processes described in U.S. Pat. Nos. 9,447,041, 9,175,320, and 8,853,419. As such, in some embodiments, the SIPR inhibitor provided herein for the therapy comprises compounds made by process described in U.S. Pat. Nos. 9,447,041, 9,175,320, and 8,853,419, the disclosures of all of which are hereby incorporated in their entireties by reference.

In certain embodiments, the S1PR inhibitor comprises an etrasimod or a derivative of etrasimod as described in Sandborn W J et al., Gastroenterology 2020 February; 158 (3): 550-561. In one embodiment, the S1PR inhibitor comprises an etrasimod or a derivative of etrasimod as described in Sandborn W J et al., Gastroenterology 2020 February; 158 (3): 550-561, at a formulation and dose as described in the same Sandborn W J publication, the disclosures of all of which are hereby incorporated in their entireties by reference.

In certain embodiments, the S1PR inhibitor comprises an ozanimod or a derivative of ozanimod selected from those described in U.S. Pat. Nos. 10,239,846, 8,796,318, and 8,481,573. In one embodiment, the S1PR inhibitor comprises an ozanimod or a derivative of ozanimod selected from those described in U.S. Pat. Nos. 10,239,846, 8,796,318, and 8,481,573, at a formulation and dose as described in U.S. Pat. Nos. 10,239,846, 8,796,318, and 8,481,573, the disclosures of all of which are hereby incorporated in their entireties by reference.

In certain embodiments, the SIPR inhibitor comprises a ponesimod or a derivative of ponesimod selected from those described in US Patent Nos. RE43728, 10220023, 9062014, 9000018, and 8273779. In one embodiment, the SPR inhibitor comprises a ponesimod or a derivative of ponesimod selected from those described in US Patent Nos. RE43728, 10220023, 9062014, 9000018, and 8273779, at a formulation and dose as described in US Patent Nos. RE43728, 10220023, 9062014, 9000018, and 8273779, the disclosures of all of which are hereby incorporated in their entireties by reference.

In certain embodiments, the SIPR inhibitor comprises an amiselimod or a derivative of amiselimod selected from those described in U.S. Pat. No. 8,809,304. In one embodiment, the S1PR inhibitor comprises an amiselimod or a derivative of amiselimod selected from those described in U.S. Pat. No. 8,809,304, at a formulation and dose as described in U.S. Pat. No. 8,809,304, the disclosures of all of which are hereby incorporated in their entireties by reference.

In certain embodiments, the S1PR inhibitor comprises an amiselimod or a derivative of amiselimod as described in Sugahara K et al., Br J Pharmacol. 2017 January; 174 (1): 15-27 and in clinical trial NCT04857112 of clinicaltrials.gov. In one embodiment, the SIPR inhibitor comprises an amiselimod or a derivative of amiselimod as described in Sugahara K et al., Br J Pharmacol. 2017 January; 174 (1): 15-27 and in clinical trial NCT04857112 of clinicaltrials.gov, at a formulation and dose as described in the same publications, the disclosures of all of which are hereby incorporated in their entireties by reference.

In certain embodiments, the S1PR inhibitor comprises a ceralifimod or a derivative of ceralifimod selected from those described in U.S. Pat. No. 7,906,549. In one embodiment, the S1PR inhibitor comprises a ceralifimod or a derivative of ceralifimod selected from those described in U.S. Pat. No. 7,906,549, at a formulation and dose as described in U.S. Pat. No. 7,906,549, the disclosures of all of which are hereby incorporated in their entireties by reference.

In certain embodiments, the SIPR inhibitor comprises a ceralifimod or a derivative of ceralifimod as described in Kurata H et al., J. Med. Chem. 2017, 60, 23, 9508-9530, Amit Bar-Or et al., Neurology Apr. 8, 2014; 82 (10 Supplement), Krosser S et al., J Clin Pharmacol. 2015 September; 55 (9): 1051-60, and in clinical trial NCT01226745 of clinicaltrials.gov. In one embodiment, the S1PR inhibitor comprises a ceralifimod or a derivative of ceralifimod as described in Kurata H et al., J. Med. Chem. 2017, 60, 23, 9508-9530, Amit Bar-Or et al., Neurology Apr. 8, 2014; 82 (10 Supplement), Krosser S et al., J Clin Pharmacol. 2015 September; 55 (9): 1051-60, and in clinical trial NCT01226745 of clinicaltrials.gov, at a formulation and dose as described in the same publications, the disclosures of all of which are hereby incorporated in their entireties by reference.

In certain embodiments, the S1PR inhibitor comprises a mocravimod or a derivative of mocravimod selected from those described in US Publication No. US20180153911A1. In one embodiment, the S1PR inhibitor comprises a mocravimod or a derivative of mocravimod selected from those described in US Publication No. US20180153911A1, at a formulation and dose as described in US Publication No. US20180153911A1, the disclosures of all of which are hereby incorporated in their entireties by reference.

In certain embodiments, the SIPR inhibitor comprises a mocravimod or a derivative of mocravimod as described in Shimizu H et al., Circulation 2005 Jan. 18; 111 (2): 222-9, Heinfried H Radeke, Inflamm Intest Dis. 2020 November; 5 (4): 180-190, Song J. et al., J Pharmacol Exp Ther. 2008; 324:276-283, Khattar M et al., Transplantation 2013 Apr. 15; 95 (7): 919-927, and in clinical trials NCT01294774 and NCT01375179 of clinicaltrials.gov. In one embodiment, the S1PR inhibitor comprises a mocravimod or a derivative of mocravimod as described in Shimizu H et al., Circulation 2005 Jan. 18; 111 (2): 222-9, Heinfried H Radeke, Inflamm Intest Dis. 2020 November; 5 (4): 180-190, Song J. et al., J Pharmacol Exp Ther. 2008; 324:276-283, Khattar M et al., Transplantation 2013 Apr. 15; 95 (7): 919-927, and in clinical trials NCT01294774 and NCT01375179 of clinicaltrials.gov, at a formulation and dose as described in the same publications, the disclosures of all of which are hereby incorporated in their entireties by reference.

In some embodiments, the S1PR inhibitor provided herein comprises anti-S1PR antibodies or antigen-binding fragments thereof. In certain embodiments, the S1PR inhibitor provided herein comprises anti-SIPR antibodies or antigen-binding fragments thereof that are specific for S1P1. In one embodiment, the S1PR inhibitor provided herein y comprises anti-SIPR antibodies or antigen-binding fragments thereof, wherein the anti-SIPR antibodies or antigen-binding fragments thereof are functional antagonists. In one embodiment, the SIPR inhibitor provided herein y comprises anti-SIPR antibodies or antigen-binding fragments thereof, wherein the anti-SIPR antibodies or antigen-binding fragments thereof are antagonist of SIPR. In one embodiment, the S1PR inhibitor provided herein comprises anti-SIPR antibodies or antigen-binding fragments thereof, wherein the anti-SIPR antibodies or antigen-binding fragments thereof inhibits binding of SIP to SIPR.

### TLIA Inhibitors

In some embodiments, the therapeutic agent comprises a modulator and/or antagonist of TNF Superfamily Member 15 (TL1A), or the gene encoding TL1A (TNFSF15). In some embodiments, the TLIA antagonist of inhibitor comprises tulisokibart, RVT-3101/PF-06480605, or duvakitug, or any combination thereof.

In some embodiments, the anti-TL1A antibody comprises tulisokibart. The disclosure further provides anti-TL1A antibodies of antigen binding fragments described in U.S. Pat. Nos. 10,322,174, 10,689,439, 11,440,954, and 11,292,848, which are hereby incorporated in its entirety by reference. In some embodiments, the anti-TL1A antibody comprises RVT-3101/PF-06480605. The disclosure further provides anti-TL1A antibodies or antigen binding fragments described in U.S. Pat. No. 9,683,998, which is hereby incorporated in its entirety by reference. In some embodiments, the anti-TL1A antibody comprises duvakitug. The disclosure further provides anti-TL1A antibodies or antigen binding fragments described in U.S. Pat. Nos. 10,138,296 and 10,822,422, the disclosures of both of which are hereby incorporated in their entireties by reference.

Alternatively, the disclosure provides siRNA, shRNA, or other RNA/DNA modalities as TL1A inhibitor to reduce TL1A expression or reduce TL1A protein levels. In some embodiment, the TL1A inhibitor comprise the siRNA against TL1A mRNA described in Gonsky R. et al., Cytokine 63 (1): 36-42 (2013), which is hereby incorporated in its entirety by reference, and which siRNA reduces TL1A secretion by 50%, for example, when transfected into human monocytes. In another embodiment, the TL1A inhibitor comprise the siRNA against TL1A mRNA described in Yu M. et al., Mol Med Rep. 2016 April; 13 (4): 3265-72, which is hereby incorporated in its entirety by reference.

Additionally, the disclosure provides the soluble TLIA receptors or TL1A decoy receptors can compete with binding between TL1A and the native DR3 receptor for TL1A, and thus be used as TL1A inhibitors. In one embodiment, the TL1A inhibitor comprises a soluble DR3 protein. In another embodiment, the TL1A inhibitor comprises a variant of the soluble DR3 protein. In yet another embodiment, the TL1A inhibitor comprises a DR3-Fc fusion protein. In yet another embodiment, the TL1A inhibitor comprises a variant of the DR3-Fc fusion protein.

The disclosure further provides that the TL1A-inhibitory effects of the DR3, DR3-Fc fusion, and the variants thereof described herein have been validated in studies as further described in Levin I et al., PLoS ONE 12 (3): e0173460. doi: 10.1371/journal.pone.0173460, the disclosures of which, including the DR3, DR3-Fc fusion, and the variants thereof tested therein, the study design and study results, are incorporated hereby in their entireties by reference.

Similarly, the disclosure provides the soluble DcR3, a TL1A decoy receptor, can compete with binding between TL1A and the native DR3 receptor for TL1A, and thus be used as TL1A inhibitors. In one embodiment, the TL1A inhibitor comprises a soluble DcR3 protein. In another embodiment, the TL1A inhibitor comprises a variant of the soluble DcR3 protein. In yet another embodiment, the TL1A inhibitor comprises a DcR3-Fc fusion protein. In yet another embodiment, the TL1A inhibitor comprises a variant of the DcR3-Fc fusion protein.

In some embodiments, the TL1A inhibitor comprises the amino acid sequence of human DcR3 (accession number: NP_003814.1) or the DcR3 variants, or DcR3 fusion proteins thereof as described in WO2021049606A1, the disclosures of which, including the DcR3, DcR3-Fc fusion, and the variants thereof tested therein, the study design and study results, are incorporated hereby in their entireties by reference.

### JAK Inhibitors

In some embodiments, the inhibitor of JAK comprises upadacitinib, filgotinib, tofacitinib, abrocitinib, baricitinib, or any combination thereof. In some embodiments, the inhibitor of JAK comprises upadacitinib. In some embodiments, the inhibitor of JAK comprises filgotinib. In some embodiments, the inhibitor of JAK comprises tofacitinib. In some embodiments, the inhibitor of JAK comprises abrocitinib. In some embodiments, the inhibitor of JAK comprises baricitinib.

### IL-21 Antagonist

In some embodiments, the methods comprise administering an interleukin 21 (IL-21) antagonist to the subject. In some embodiments, the IL-21 antagonist comprises an antibody, or an antigen binding fragment thereof.

### Methods of Monitoring Treatment

In certain embodiments, described herein are methods of determining the prognosis of POR of CD in a subject comprising detecting one or more of the POR markers described herein and determining the subject to have increased likelihood of POR based, at least in part on the one or more POR markers detected in the subject, wherein the increased likelihood of POR is compared with a control subject having the CD, wherein the control subject does not have the one or more POR markers.

In certain embodiments, described herein is a method of determining prophylactic persistence of therapeutic agent in treating Crohn's disease (CD) in a subject that has undergone operation, the method comprising: detecting one or more POPP markers in the subject; and determining the therapeutic agent to have the prophylactic persistence in treating the CD in the subject based, at least in part, on the one or more POPP markers detected in the subject, wherein the CD is post-operative.

### Kits and Compositions

### Compositions

Disclosed herein are compositions useful for the detection of a genotype or biomarker in a sample obtained from a subject according to the methods described herein. Aspects disclosed herein provide compositions comprises a polynucleotide sequence comprising at least 10 but less than 50 contiguous nucleotides of rs6811007, or a polymorphism listed in Table 3 or Table 4, or reverse complements thereof, wherein the contiguous polynucleotide sequence comprises a detectable molecule. In various embodiments, the detectable molecule comprises a fluorophore. In other embodiments, the polynucleotide sequences further comprise a quencher.

Also disclosed herein are compositions comprising an antibody or antigen-binding fragment that specifically binds to a target protein described herein (e.g., TL1A) wherein the antibody or antigen-binding fragment comprises a detectable molecule. In various embodiments, the antibody comprises a monoclonal antibody, a chimeric antibody, a CDR-grafted antibody, a Fab, a Fab′, a F(ab′)2, a Fv, a disulfide linked Fv, a scFv, a single domain antibody, a diabody, a multispecific antibody, a dual specific antibody, an anti-idiotypic antibody, or a bispecific antibody. In some embodiments, the antibody or antigen-binding fragment comprises an IgG antibody, an IgM antibody, and/or an IgE antibody. In some embodiments, the detectable molecule comprises a fluorophore. In some embodiments, the antibody or antigen-binding fragment is conjugated to a paramagnetic particle (e.g., bead).

### Kits

Disclosed herein, are kits useful for to detect the genotypes and/or biomarkers disclosed herein. In some embodiments, the kits disclosed herein may be used to diagnose and/or treat a disease or condition in a subject; or select a patient for treatment and/or monitor a treatment disclosed herein. In some embodiments, the kit comprises the compositions described herein, which can be used to perform the methods described herein. Kits comprise an assemblage of materials or components, including at least one of the compositions. Thus, in some embodiments the kit contains a composition including of the pharmaceutical composition, for the treatment of CD. In other embodiments, the kits contains all of the components necessary and/or sufficient to perform an assay for detecting and measuring CD markers, including all controls, directions for performing assays, and any necessary software for analysis and presentation of results.

In some embodiments, the kits described herein comprise components for detecting the presence, absence, and/or quantity of a target nucleic acid and/or protein described herein. In some embodiments, the kit further comprises components for detecting the presence, absence, and/or quantity of a serological marker described herein. In some embodiments, the kit comprises the compositions (e.g., primers, probes, antibodies) described herein. The disclosure provides kits suitable for assays such as enzyme-linked immunosorbent assay (ELISA), single-molecular array (Simoa), PCR, and qPCR. The exact nature of the components configured in the kit depends on its intended purpose. In some embodiments, the kit may comprise a detection agent configured to bind configured to bind or hybridize a nucleic acid encoding IL21|BBS12 at a polymorphism position comprising rs6811007 or a proxy polymorphism position in linkage disequilibrium therewith as determined with an r² of at least 0.85, or a combination thereof. In some embodiments, the kit may comprise a detection agent configured to bind configured to bind or hybridize a nucleic acid at a position listed in Table 3 or Table 4 or a proxy polymorphism position in linkage disequilibrium therewith as determined with an r² of at least 0.85, or a combination thereof. In some embodiments, the kit comprises a detection agent configured to bind ANCA, ASCA IgA, ASCA IgG, or combinations thereof.

In some embodiments, the kits described herein are configured for the purpose of treating and/or characterizing a disease or condition (e.g., Crohn's disease), or subclinical phenotype thereof (e.g., stricturing, penetrating, or stricturing and penetrating disease phenotypes) in a subject. In some embodiments, the kits described herein are configured for the purpose of identifying a subject with a high likelihood of post-operative recurrence. In some embodiments, the kits described herein are configured for the purpose of identifying a subject with a high likelihood of post-operative prophylaxis persistence. In some embodiments, the kit is configured particularly for the purpose of treating mammalian subjects. In some embodiments, the kit is configured particularly for the purpose of treating human subjects. In further embodiments, the kit is configured for veterinary applications, treating subjects such as, but not limited to, farm animals, domestic animals, and laboratory animals. In some embodiments, the kit is configured to select a subject for a therapeutic agent, such as those disclosed herein. In some embodiments, the kit is configured to select a subject for treatment with a therapeutic agent disclosed herein. In one embodiment, a therapeutic agent is a TNF inhibitor, an IL23 inhibitor, an integrin inhibitor, a SIPR inhibitor, a TL1A inhibitor, a JAK inhibitor, or an IL21 inhibitor.

Instructions for use may be included in the kit. Instructions may comprise instructions for measuring a POPP marker or a POR marker, instructions for treatment of a subject, instructions for selection of a subject, instructions for selection of a therapeutic agent, or instructions for a diagnosis of a subject. In some embodiments, the kit comprises instructions for predicting the POPP or POR in the subject.

In some embodiments, the kit comprises instructions for reviewing of medical records of the subject for POPP markers. In some embodiments, the kit comprises instructions for identifying the subject as having a low likelihood of the POPP, wherein the one or more POPP clinical factors detected in the subject comprises an active smoker status. In some embodiments, the kit comprises instructions for identifying the subject as having a high likelihood of the POPP, wherein the one or more POPP clinical factors detected in the subject comprises a disease location in an ileal region of an intestine of the subject. In some embodiments, the kit comprises instructions for identifying the subject as having a low likelihood of the POPP, wherein the one or more POPP clinical factors detected in the subject comprises a disease location in an ileocolonic region of an intestine of the subject. In some embodiments, the kit comprises instructions for identifying the subject as having a low likelihood of the POPP, wherein the one or more POPP clinical factors detected in the subject comprises a lymph node granuloma. In some embodiments, the kit comprises instructions for identifying the subject as having a low likelihood of the POPP, wherein the one or more POPP clinical factors detected in the subject comprises one or more prior treatment failures, the prior treatments comprising a steroid, an immunomodulator, 5-aminosalicylate, or combinations thereof. In some embodiments, the kit comprises instructions for identifying the subject as having a low likelihood of the POPP, wherein the one or more POPP clinical factors detected in the subject comprises one or more prior biologic failures. In some embodiments, the kit comprises instructions for identifying the subject as having a low likelihood of the POPP, wherein the one or more POPP clinical factors detected in the subject comprises two or more prior biologic failures. In some embodiments, the kit comprises instructions for identifying the subject as having a low likelihood of the POPP, wherein the one or more POPP clinical factors detected in the subject comprises three or more prior biologic failures. In some embodiments, the kit comprises instructions for identifying the subject as having a high likelihood of POPP, wherein the one or more POPP serological factors detected in the subject comprises ASCA seropositivity. In some embodiments, the kit comprises instructions for identifying the subject as having a high likelihood of POPP wherein the one or more POPP serological factors detected in the subject comprises ANCA seronegativity. In some embodiments, the kit comprises instructions for identifying the subject as having a high likelihood of POPP if the subject wherein the one or more POPP serological factors detected in the subject comprises ASCA seropositivity and ANCA seronegativity. In some embodiments, the kit comprises instructions for identifying the subject as having a high likelihood of the POPP, wherein the one or more POPP clinical factors detected in the subject comprises operative visceral adipose tissue (VAT) quality having one or more of: a VAT radiodensity of less than or equal to about 0.631 Hounsfield unit (HU); a VAT radiodensity of about 0.43 to about 0.7 HU; a reduced radiodensity comprises a ratio of VAT to SAT radiodensity of up to 5.559; or a ratio of VAT to SAT radiodensity of ranging from 1.18-6. In some embodiments, the kit comprises instructions for identifying the subject as having a high likelihood of POPP if a nucleic acid encoding IL21|BBS12 at a polymorphism position comprising rs6811007 or a proxy polymorphism position in linkage disequilibrium therewith as determined with an r² of at least 0.85, or a combination thereof is not detected. In some embodiments, the kit comprises instructions for identifying the subject as having a high likelihood of POPP if a nucleic acid listed in Table 3 or Table 4 a proxy polymorphism position in linkage disequilibrium therewith as determined with an r² of at least 0.85, or a combination thereof is not detected. In some embodiments, the kit comprises instructions for identifying the subject as having a high likelihood of POPP if a nucleic acid encoding IL21|BBS12 at a polymorphism position comprising rs 1533236 or a proxy polymorphism position in linkage disequilibrium therewith as determined with an r² of at least 0.85, or a combination thereof is not detected.

In some embodiments, the kit comprises instructions for reviewing of medical records of the subject for POR markers. In some embodiments, the kit comprises instructions for identifying the subject as having a high likelihood of the POR, wherein the one or more POR clinical factors detected in the subject comprises an active smoker status. In some embodiments, the kit comprises instructions for identifying the subject as having a low likelihood of the POR, wherein the one or more POR clinical factors detected in the subject comprises a disease location in an ileal region of an intestine of the subject. In some embodiments, the kit comprises instructions for identifying the subject as having a high likelihood of the POR, wherein the one or more POR clinical factors detected in the subject comprises a disease location in an ileocolonic region of an intestine of the subject. In some embodiments, the kit comprises instructions for identifying the subject as having a high likelihood of the POR, wherein the one or more POR clinical factors detected in the subject comprises a lymph node granuloma. In some embodiments, the kit comprises instructions for identifying the subject as having a high likelihood of the POR, wherein the one or more POR clinical factors detected in the subject comprises one or more prior treatment failures, the prior treatments comprising a steroid, an immunomodulator, 5-aminosalicylate, or combinations thereof. In some embodiments, the kit comprises instructions for identifying the subject as having a high likelihood of the POR, wherein the one or more POR clinical factors detected in the subject comprises one or more prior biologic failures. In some embodiments, the kit comprises instructions for identifying the subject as having a high likelihood of the POR, wherein the one or more POR clinical factors detected in the subject comprises two or more prior biologic failures. In some embodiments, the kit comprises instructions for identifying the subject as having a high likelihood of the POR, wherein the one or more POR clinical factors detected in the subject comprises three or more prior biologic failures. In some embodiments, the kit comprises instructions for identifying the subject as having a low likelihood of POR, wherein the one or more POR serological factors detected in the subject comprises ASCA seropositivity. In some embodiments, the kit comprises instructions for identifying the subject as having a low likelihood of POR wherein the one or more POR serological factors detected in the subject comprises ANCA seronegativity. In some embodiments, the kit comprises instructions for identifying the subject as having a low likelihood of POR wherein the one or more POR serological factors detected in the subject comprises ASCA seropositivity and ANCA seronegativity. In some embodiments, the kit comprises instructions for identifying the subject as having a low likelihood of the POR, wherein the one or more POR clinical factors detected in the subject comprises operative visceral adipose tissue (VAT) quality having one or more of: a VAT radiodensity of less than or equal to about 0.631 Hounsfield unit (HU); a VAT radiodensity of about 0.43 to about 0.7 HU; a reduced radiodensity comprises a ratio of VAT to SAT radiodensity of up to 5.559; or a ratio of VAT to SAT radiodensity of ranging from 1.18-6. In some embodiments, the kit comprises instructions for identifying the subject as having a high likelihood of POR if a nucleic acid encoding IL21|BBS12 at a polymorphism position comprising rs6811007 or a proxy polymorphism position in linkage disequilibrium therewith as determined with an r² of at least 0.85, or a combination thereof is not detected. In some embodiments, the kit comprises instructions for identifying the subject as having a high likelihood of POR if a nucleic acid listed in Table 3 or Table 4 or a proxy polymorphism position in linkage disequilibrium therewith as determined with an r² of at least 0.85, or a combination thereof is not detected.

Optionally, the kit also contains other useful components, such as, diluents, buffers, pharmaceutically acceptable carriers, syringes, catheters, applicators, pipetting or measuring tools, bandaging materials or other useful paraphernalia. The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as compositions and the like. The packaging material is constructed by well-known methods, preferably to provide a sterile, contaminant-free environment. The packaging materials employed in the kit are those customarily utilized in gene expression assays and in the administration of treatments. As used herein, the term “package” refers to a suitable solid matrix or material such as glass, plastic, paper, foil, and the like, capable of holding the individual kit components. Thus, for example, a package can be a glass vial or prefilled syringes used to contain suitable quantities of the pharmaceutical composition. The packaging material has an external label which indicates the contents and/or purpose of the kit and its components.

### Systems

Disclosed herein are systems for determining the prognosis of post-operative recurrence (“POR”) of Crohn's disease (CD) in a subject. Also described herein are systems for determining prophylactic persistence of a therapeutic agent in treating CD in a subject. In some embodiments, the systems described herein comprise kits and compositions for detecting the genotypes described herein in a biological sample of a subject. The system may comprise a computer system or platform.

In certain aspects, described herein is a computing platform for predicting a post-operative prophylaxis persistence (“POPP”) in a subject. In some embodiments, the platform comprises a genotyping device for determining genotype data of a subject with Crohn's disease (CD). In some embodiments, the platform comprises one or more computer processors operably coupled to the genotyping device. In some embodiments, the one or more computer processors are individually or collectively configured to perform operations comprising: receiving the genotype data from the genotyping device; and identifying, in the genotype data, a genotype that is predictive of POPP.

In certain aspects, described herein is a computing platform for predicting a post-operative recurrence (“POR”) in a subject. In some embodiments, the computing platform comprises a genotyping device for determining genotype data of a subject with Crohn's disease (CD). In some embodiments, one or more computer processors operably coupled to the genotyping device. In some embodiments, the one or more computer processors are individually or collectively configured to perform operations comprising: receiving the genotype data from the genotyping device; and identifying, in the genotype data, a predetermined genotype that is predictive of POR.

### Computer Systems

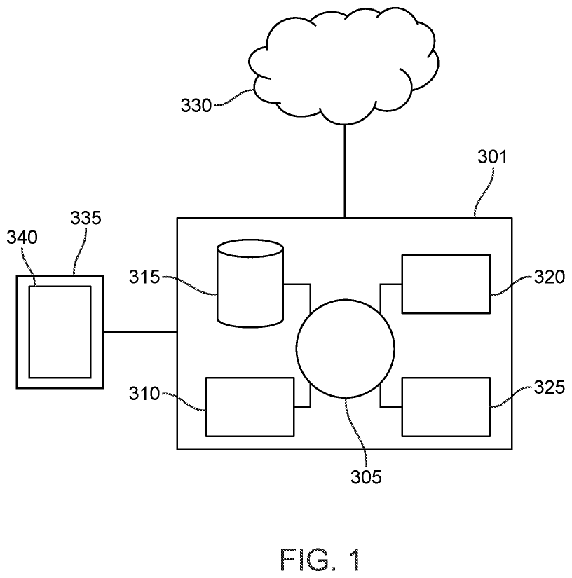

FIG. 1 shows a computer system 301 that is programmed or otherwise configured to predict a post-operative prophylaxis persistence or a post-operative recurrence in a subject for a subject in need thereof. The computer system 301 can regulate various aspects of producing the genetic risk profile (e.g., receiving genotype data, generating a report with the genetic risk profile of the biological sample, and displaying the report to a user), of the present disclosure, such as, for example, by including permissions or encryption of genotype data and/or genetic risk profile of the subject to ensure patient privacy. In some embodiments, the computer system selects one or more genetic variants. In some embodiments, the one of more genetic variants comprise one or more genotypes of the subject or a predetermined genetic variant in a linkage disequilibrium (LD) therewith. In some embodiments, the gene or genetic locus comprises IL21|BBS12. In some embodiments, the one or more genetic variants comprises rs6811007. In some embodiments, the one or more genetic variants comprises a genetic variant listed in Table 3 or Table 4. In some embodiments, the computer system calculates a genetic risk score for the subject, based, at least in part on the one or more genetic variants. In some embodiments, the one or more genetic variants comprises rs1533236.

In some embodiments, the computer system predicts post-operative prophylaxis persistence in the subject. In some embodiments, the computer system calculates a positive predictive value for the genotype to predict a high likelihood of post-operative prophylaxis persistence in the subject. In some embodiments, the computer system calculates a negative predictive value for the genotype to predict a high likelihood of post-operative prophylaxis persistence in the subject. In some embodiments, the computer system calculates a specificity for the genotype to predict a high likelihood of post-operative prophylaxis persistence in the subject.

In some embodiments, the computer system predicts post-operative recurrence in the subject. In some embodiments, the computer system calculates a positive predictive value for the genotype to predict a high likelihood of post-operative recurrence in the subject. In some embodiments, the computer system calculates a negative predictive value for the genotype to predict a high likelihood of post-operative recurrence in the subject. In some embodiments, the computer system calculates a specificity for the genotype to predict a high likelihood of post-operative recurrence in the subject. In some embodiments, the computer system calculates a sensitivity for the genotype to predict a high likelihood of post-operative recurrence in the subject.

In some embodiments, the predetermined genotype does not comprise rs6811007, or a proxy polymorphism in linkage disequilibrium therewith as determined with an R2 of at least 0.85, or a combination thereof. In some embodiments, the predetermined genotype does not comprise a genotype listed in Table 3 or Table 4. In some embodiments, the high likelihood of POPP is measured as compared with a control sample obtained from a control subject diagnosed with CD and having undergone a recission operation and not comprising a rs6811007 genotype.

In some embodiments, the predetermined genotype comprises rs6811007, or a proxy polymorphism in linkage disequilibrium therewith as determined with an R2 of at least 0.85, or a combination thereof. In some embodiments, the high likelihood of POR is measured as compared with a control sample obtained from a control subject diagnosed with CD and having undergone a recission operation and comprising a rs6811007 genotype. In some embodiments, the predetermined genotype comprises a genotype listed in Table 3 or Table 4.

The computer system 301 can be an electronic device of a user or a computer system that is remotely located with respect to the electronic device. The electronic device can be a mobile electronic device, such as a mobile electronic device belonging to a physician.

The computer system 301 includes a central processing unit (CPU, also “processor” and “computer processor” herein) 305, which can be a single core or multi core processor, or a plurality of processors for parallel processing. The computer system 301 also includes memory or memory location 310 (e.g., random-access memory, read-only memory, flash memory), electronic storage unit 315 (e.g., hard disk), communication interface 320 (e.g., network adapter) for communicating with one or more other systems, and peripheral devices 325, such as cache, other memory, data storage and/or electronic display adapters. The memory 310, storage unit 315, interface 320 and peripheral devices 325 are in communication with the CPU 305 through a communication bus (solid lines), such as a motherboard. The storage unit 315 can be a data storage unit (or data repository) for storing data. The computer system 301 can be operatively coupled to a computer network (“network”) 330 with the aid of the communication interface 320. The network 330 can be the Internet, an internet and/or extranet, or an intranet and/or extranet that is in communication with the Internet. The network 330 in some cases is a telecommunication and/or data network. The network 330 can include one or more computer servers, which can enable distributed computing, such as cloud computing. The network 330, in some cases with the aid of the computer system 301, can implement a peer-to-peer network, which may enable devices coupled to the computer system 301 to behave as a client or a server.

The CPU 305 can execute a sequence of machine-readable instructions, which can be embodied in a program or software. The instructions may be stored in a memory location, such as the memory 310. The instructions can be directed to the CPU 305, which can subsequently program or otherwise configure the CPU 305 to implement methods of the present disclosure. Examples of operations performed by the CPU 305 can include fetch, decode, execute, and writeback.

The CPU 305 can be part of a circuit, such as an integrated circuit. One or more other components of the system 301 can be included in the circuit. In some cases, the circuit is an application specific integrated circuit (ASIC).

The storage unit 315 can store files, such as drivers, libraries and saved programs. The storage unit 315 can store user data, e.g., user preferences and user programs. The computer system 301 in some cases can include one or more additional data storage units that are external to the computer system 301, such as located on a remote server that is in communication with the computer system 301 through an intranet or the Internet.

The computer system 301 can communicate with one or more remote computer systems through the network 330. For instance, the computer system 301 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 301 via the network 330.

Methods as described herein can be implemented by way of machine (e.g., computer processor) executable code stored on an electronic storage location of the computer system 301, such as, for example, on the memory 310 or electronic storage unit 315. The machine executable or machine readable code can be provided in the form of software. During use, the code can be executed by the processor 305. In some cases, the code can be retrieved from the storage unit 315 and stored on the memory 310 for ready access by the processor 305. In some situations, the electronic storage unit 315 can be precluded, and machine-executable instructions are stored on memory 310.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 301, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 301 can include or be in communication with an electronic display 335 that comprises a user interface (UI) 340 for providing, for example, a report comprising the genetic risk profile of the subject or other relevant clinical information for purposes of informing a selection of a therapeutic agent (e.g., anti-TL1A antibody) to treat a disease or condition of the subject described herein. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 305. The algorithm can, for example, perform: (a) receiving genotype data of a subject, (b) determining whether the genotypes are heterozygous or homozygous for nine polymorphisms, (c) generating an outcome using predetermined parameters, and (d) displaying the outcome to a user (e.g., physician) on a user interface of an electronic device. In some embodiments, the outcome is positive, negative or indeterminant. In some embodiments, the predetermined parameters are genotype combinations known to be predictive of a therapeutic response to a treatment, such as with an inhibitor of TL1A activity or expression.

### Web Application

In some embodiments, the computer system comprises software for a web application. In light of the disclosure provided herein, in some embodiments, a web application may utilize one or more software frameworks and one or more database systems. A web application, for example, is created upon a software framework such as Microsoft® NET or Ruby on Rails (RoR). A web application, In some embodiments, utilizes one or more database systems including, by way of non-limiting examples, relational, non-relational, feature oriented, associative, and XML database systems. Suitable relational database systems include, by way of non-limiting examples, Microsoft® SQL Server, mySQL™, and Oracle®. In some embodiments, a web application may be written in one or more versions of one or more languages. In some embodiments, a web application is written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). A web application may integrate enterprise server products such as IBM® Lotus Domino®. A web application may include a media player element. A media player element may utilize one or more of many suitable multimedia technologies including, by way of non-limiting examples, Adobe® Flash®, HTML 5, Apple® QuickTime®, Microsoft® Silverlight®, Java™, and Unity®.

### Mobile Application

In some embodiments, the computer system comprises software for a mobile application. The mobile application may be provided to a mobile digital processing device at the time it is manufactured. The mobile application may be provided to a mobile digital processing device via the computer network described herein.

A mobile application is created by using hardware, languages, and development environments. In some embodiments, the mobile applications may be written in several languages. Suitable programming languages include, by way of non-limiting examples, C, C++, C#, Featureive-C, Java™, Javascript, Pascal, Feature Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments may be available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, the computer system comprises software a standalone application, which is a program that may be run as an independent computer process, not an add-on to an existing process, e.g., not a plug-in. In some embodiments, standalone applications are sometimes compiled. In some embodiments, a compiler is a computer program(s) that transforms source code written in a programming language into binary feature code such as assembly language or machine code. Suitable compiled programming languages include, by way of non-limiting examples, C, C++, Featureive-C, COBOL, Delphi, Eiffel, Java™, Lisp, Python™, Visual Basic, and VB.NET, or combinations thereof. Compilation may be often performed, at least in part, to create an executable program. In some embodiments, a computer program includes one or more executable complied applications.

### Web Browser Plug-In

In some embodiments, the computer system comprises software that comprises a web browser plug-in. In some embodiments, in computing, a plug-in is one or more software components that add specific functionality to a larger software application. Makers of software applications may support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Examples of web browser plug-ins include, without limitations, Adobe® Flash® Player, Microsoft® Silverlight®, and Apple® QuickTime®. The toolbar may comprise one or more web browser extensions, add-ins, or add-ons. The toolbar may comprise one or more explorer bars, tool bands, or desk bands.

In view of the disclosure provided herein, plug-in frameworks are available that enable development of plug-ins in various programming languages, include, by way of non-limiting examples, C++, Delphi, Java™, PHP, Python™, and VB.NET, or combinations thereof.

In some embodiments, Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google Chrome, Apple® Safari®, Opera Software® Opera®, and KDE Konqueror. In some embodiments, the web browser is a mobile web browser. Mobile web browsers (also called microbrowsers, mini-browsers, and wireless browsers) may be designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM Blackberry® Browser, Apple® Safari®, Palm® Blazer, Palm® WebOS® Browser, Mozilla Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

The mediums, methods, and systems disclosed herein may comprise one or more softwares, servers, and database modules, or use of the same. In view of the disclosure provided herein, software modules may be created by using machines, software, and languages. The software modules disclosed herein may be implemented in a multitude of ways. In some embodiments, a software module comprises a file, a section of code, a programming feature, a programming structure, or combinations thereof. A software module may comprise a plurality of files, a plurality of sections of code, a plurality of programming features, a plurality of programming structures, or combinations thereof. By way of non-limiting examples, the one or more software modules comprise a web application, a mobile application, and/or a standalone application. Software modules may be in one computer program or application. Software modules may be in more than one computer program or application. Software modules may be hosted on one machine. Software modules may be hosted on more than one machine. Software modules may be hosted on cloud computing platforms. Software modules may be hosted on one or more machines in one location. Software modules may be hosted on one or more machines in more than one location.

### Databases

The mediums, methods, and systems disclosed herein may comprise one or more databases, or use of the same. In view of the disclosure provided herein, some databases are suitable for storage and retrieval of geologic profile, operator activities, division of interest, and/or contact information of royalty owners may be used. Suitable databases include, by way of non-limiting examples, relational databases, non-relational databases, feature oriented databases, feature databases, entity-relationship model databases, associative databases, and XML databases. In some embodiments, a database is internet-based. In some embodiments, a database is web-based. In some embodiments, a database is cloud computing-based. A database may be based on one or more local computer storage devices.

### Data Transmission

The subject matter described herein, including methods for producing a genetic risk profile are configured to be performed in one or more facilities at one or more locations. Facility locations are not limited by country and include any country or territory. In some embodiments, one or more steps are performed in a different country than another step of the method. In some embodiments, one or more steps for obtaining a sample are performed in a different country than one or more steps for detecting the presence or absence of a genotype in a biological sample. In some embodiments, one or more method steps involving a computer system are performed in a different country than another step of the methods provided herein. In some embodiments, data processing and analyses are performed in a different country or location than one or more steps of the methods described herein. In some embodiments, one or more articles, products, or data are transferred from one or more of the facilities to one or more different facilities for analysis or further analysis. An article includes, but is not limited to, one or more components obtained from a subject, e.g., processed cellular material. Processed cellular material includes, but is not limited to, cDNA reverse transcribed from RNA, amplified RNA, amplified cDNA, sequenced DNA, isolated and/or purified RNA, isolated and/or purified DNA, and isolated and/or purified polypeptide. Data includes, but is not limited to, information regarding the stratification of a subject, and any data produced by the methods disclosed herein. In some embodiments of the methods and systems described herein, the analysis is performed and a subsequent data transmission step will convey or transmit the results of the analysis.

In some embodiments, any step of any method described herein is performed by a software program or module on a computer. In additional or further embodiments, data from any step of any method described herein is transferred to and from facilities located within the same or different countries, including analysis performed in one facility in a particular location and the data shipped to another location or directly to an individual in the same or a different country. In additional or further embodiments, data from any step of any method described herein is transferred to and/or received from a facility located within the same or different countries, including analysis of a data input, such as genetic or processed cellular material, performed in one facility in a particular location and corresponding data transmitted to another location, or directly to an individual, such as data related to the diagnosis, prognosis, responsiveness to therapy (e.g., anti-TL1A therapy), or the like, in the same or different location or country.

### Business Methods Utilizing a Computer

The methods described herein may utilize one or more computers. The computer may be used for managing customer and biological sample information such as sample or customer tracking, database management, analyzing molecular profiling data, analyzing cytological data, storing data, billing, marketing, reporting results, storing results, or a combination thereof. The computer may include a monitor or other user interface for displaying data, results, billing information, marketing information (e.g., demographics), customer information, or sample information. The computer may also include means for data or information input. The computer may include a processing unit and fixed or removable media or a combination thereof. The computer may be accessed by a user in physical proximity to the computer, for example via a keyboard and/or mouse, or by a user that does not necessarily have access to the physical computer through a communication medium such as a modem, an internet connection, a telephone connection, or a wired or wireless communication signal carrier wave. In some cases, the computer may be connected to a server or other communication device for relaying information from a user to the computer or from the computer to a user. In some cases, the user may store data or information obtained from the computer through a communication medium on media, such as removable media. It is envisioned that data relating to the methods can be transmitted over such networks or connections for reception and/or review by a party. The receiving party can be but is not limited to an individual, a health care provider (e.g., physician) or a health care manager. In one embodiment, a computer-readable medium includes a medium suitable for transmission of a result of an analysis of a biological sample, such as exosome bio-signatures. The medium can include a result regarding an exosome bio-signature of a subject, wherein such a result is derived using the methods described herein.

The entity obtaining a report with the genetic risk profile may enter biological sample information into a database for the purpose of one or more of the following: inventory tracking, assay result tracking, order tracking, customer management, customer service, billing, and sales. Sample information may include, but is not limited to: customer name, unique customer identification, customer associated medical professional, indicated assay or assays, assay results, adequacy status, indicated adequacy tests, medical history of the individual, preliminary diagnosis, suspected diagnosis, sample history, insurance provider, medical provider, third party testing center or any information suitable for storage in a database. Sample history may include but is not limited to: age of the sample, type of sample, method of acquisition, method of storage, or method of transport.

The database may be accessible by a customer, medical professional, insurance provider, or other third party. Database access may take the form of electronic communication such as a computer or telephone. The database may be accessed through an intermediary such as a customer service representative, business representative, consultant, independent testing center, or medical professional. The availability or degree of database access or sample information, such as assay results, may change upon payment of a fee for products and services rendered or to be rendered. The degree of database access or sample information may be restricted to comply with generally accepted or legal requirements for patient or customer confidentiality.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a,” “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87:2264-2268, 1990, modified as in Proc. Natl. Acad. Sci. USA 90:5873-5877, 1993). Such a formula is incorporated into the basic local alignment search tool (BLAST) programs of Altschul et al. (J Mol Biol. 1990 Oct. 5; 215 (3): 403-10; Nucleic Acids Res. 1997 Sep. 1; 25 (17): 3389-402). Percent homology of sequences can be determined using the most recent version of BLAST, as of the filing date of this application. Percent identity of sequences can be determined using the most recent version of BLAST, as of the filing date of this application.

The terms “increased,” or “increase” are used herein to generally mean an increase by a statically significant amount. In some embodiments, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level. An increase can be an absolute amount (e.g., level of protein expression), or a rate of production (e.g., rate of protein expression between two points in time).

The terms “decreased” or “decrease” are used herein generally to mean a decrease by a statistically significant amount. In some embodiments, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. Other examples of “increase” include an increase of at least 2-fold, at least 5-fold, at least 10-fold, at least 20-fold, at least 50-fold, at least 100-fold, at least 1000-fold or more as compared to a reference level. An increase can be an absolute amount (e.g., level of protein expression), or a rate of production (e.g., rate of protein expression between two points in time). In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease. Other examples of “decrease” include a decrease of at least 2-fold, at least 5-fold, at least 10-fold, at least 20-fold, at least 50-fold, at least 100-fold, at least 1000-fold or more as compared to a reference level. A decrease can be an absolute amount (e.g., level of protein expression), or a rate of production (e.g., rate of protein expression between two points in time).

The terms “subject” encompass mammals. Non-limiting examples of mammal include any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human. The term “animal” as used herein comprises human beings and non-human animals. In one embodiment, a “non-human animal” is a mammal, for example a rodent such as rat or a mouse. In some embodiments, a human subject is a “patient,” which as used herein, refers to a subject who has or may be diagnosed with a disease or condition disclosed herein.

The term “gene,” as used herein, refers to a segment of nucleic acid that encodes an individual protein or RNA (also referred to as a “coding sequence” or “coding region”), optionally together with associated regulatory region such as promoter, operator, terminator and the like, which may be located upstream or downstream of the coding sequence. A “genetic locus” referred to herein, is a particular location within a gene.

The term, “genotype” as disclosed herein, refers to the chemical composition of a polynucleotide sequence within the genome of an individual. In some embodiments, the genotype comprises a single nucleotide polymorphism (SNP) or and indel (insertion or deletion, of a nucleobase within a polynucleotide sequence). In some embodiments, a genotype for a particular SNP, or indel is heterozygous. In some embodiments, a genotype for a particular SNP, or indel is homozygous.

A “polymorphism” as used herein refers to an aberration in (e.g., a mutation), or of (e.g., insertion/deletion), a nucleic acid sequence, as compared to the nucleic acid sequence in a reference population. In some embodiments, the polymorphism is common in the reference population. In some embodiments, the polymorphism is rare in the reference population. In some embodiments, the polymorphism is a single nucleotide polymorphism.

The term, “single nucleotide polymorphism” or SNP as disclosed herein, refers to a variation in a single nucleotide within a polynucleotide sequence. The term should not be interpreted as placing a restriction on a frequency of the SNP in a given population. The variation of an SNP may have multiple different forms. A single form of an SNP is referred to as an “allele.” An SNP can be mono-, bi-, tri, or tetra-allelic. A SNP may include a “risk allele,” a “protective allele,” or neither. By way of example, a reference polynucleotide sequence reading 5′ to 3′ is TTACG. A SNP at allele position 3 (of 5′-TTACG-3′) comprise a substitution of the reference allele, “A” to a non-reference allele, “C.” If the “C” allele of the SNP is associated with an increased probability of developing a phenotypic trait, the allele is considered a “risk” allele. However, the same SNP may also comprise a substitution of the “A” allele to a “T” allele at position 3. If the T allele of the SNP is associated with a decreased probability of developing a phenotypic trait, the allele is considered a “protective” allele. The SNP may be observed in at least 1% of a given population. In some embodiments, the SNP is represented by an “rs” number, which refers to the accession of reference cluster of one more submitted SNPs in the dbSNP bioinformatics database as of the filing date of this patent application, and which is included within a sequence that comprises the total number of nucleobases from 5′ to 3′. In some embodiments, a SNP may be further defined by the position of the SNP (nucleobase) within the dbSNP sequence, the position of which is always with reference to 5′ length of the sequence plus 1. In some embodiments, a SNP is defined as the genomic position in a reference genome and the allele change (e.g. chromosome 7 at position 234,123,567 from G allele to A allele in the reference human genome build 37). In some embodiments, the SNV is defined as the genomic position identified with [brackets] or an “N” in a sequence disclosed herein.

The term, “indel,” as disclosed herein, refers to an insertion, or a deletion, of a nucleobase within a polynucleotide sequence. An indel can be mono-, bi-, tri, or tetra-allelic. An indel may be “risk,” a “protective,” or neither, for a phenotypic trait. In some embodiments, the indel is represented by an “rs” number, which refers to the accession of reference cluster of one more submitted indels in the dbSNP bioinformatics database as of the filing date of this patent application, and which is included in a sequence that comprises the total number of nucleobases from 5′ to 3′. In some embodiments, an indel may be further defined by the position of the insertion/deletion within the dbSNP sequence, the position of which is always with reference to the 5′ length of the sequence plus 1. In some embodiments, an indel is defined as the genomic position in a reference genome and the allele change. In some embodiments, the indel is defined as the genomic position identified with [brackets] or an “N” in a sequence disclosed herein.

“Haplotype” as used herein, encompasses a group of one or more genotypes, which tend to be inherited together in a reference population. In some embodiments, a haplotype comprises particular polymorphism or another polymorphism in linkage disequilibrium (LD) therewith.

“Linkage disequilibrium,” or “LD,” as used herein refers to the non-random association of alleles or indels in different gene loci in a given population. LD may be defined by a D′ value corresponding to the difference between an observed and expected allele or indel frequencies in the population (D=Pab-PaPb), which is scaled by the theoretical maximum value of D. LD may be defined by an r2 value corresponding to the difference between an observed and expected unit of risk frequencies in the population (D=Pab-PaPb), which is scaled by the individual frequencies of the different loci. In some embodiments, D′ comprises at least 0.20. In some embodiments, r2 comprises at least 0.70.

The term “medically refractory,” or “refractory,” as used herein, refers to the failure of a standard treatment to induce remission of a disease. In some embodiments, the disease comprises an inflammatory disease disclosed herein. A non-limiting example of refractory inflammatory disease includes refractory Crohn's disease, and refractory ulcerative colitis (e.g., mrUC). Non-limiting examples of standard treatment include glucocorticosteriods, anti-TNF therapy, anti-a4-b7 therapy (vedolizumab), anti-IL12p40 therapy (ustekinumab), Thalidomide, and Cytoxin.

The terms “treat,” “treating,” and “treatment” as used herein refers to alleviating or abrogating a disorder, disease, or condition; or one or more of the symptoms associated with the disorder, disease, or condition; or alleviating or eradicating a cause of the disorder, disease, or condition itself. Desirable effects of treatment can include, but are not limited to, preventing occurrence or recurrence of disease, alleviation of symptoms, diminishing any direct or indirect pathological consequences of the disease, preventing metastasis, decreasing the rate of disease progression, amelioration or palliation of the disease state and remission or improved prognosis.

The term “therapeutically effective amount” refers to the amount of a compound or therapy that, when administered, is sufficient to prevent development of, or alleviate to some extent, one or more of the symptoms of a disorder, disease, or condition of the disease; or the amount of a compound that is sufficient to elicit biological or medical response of a cell, tissue, system, animal, or human that is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. A component can be “pharmaceutically acceptable” in the sense of being compatible with the other ingredients of a pharmaceutical formulation. It can also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, PA, 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, FL, 2004).

The term “pharmaceutical composition” refers to a mixture of a compound disclosed herein with other chemical components, such as diluents or carriers. The pharmaceutical composition can facilitate administration of the compound to an organism. Multiple techniques of administering a compound exist in the art including, but not limited to, oral, injection, aerosol, parenteral, and topical administration.

The term “inflammatory bowel disease” or “IBD” as used herein refers to gastrointestinal disorders of the gastrointestinal tract. Non-limiting examples of IBD include, Crohn's disease (CD), ulcerative colitis (UC), indeterminate colitis (IC), microscopic colitis, diversion colitis, Behcet's disease, and other inconclusive forms of IBD. In some embodiments, IBD comprises fibrosis, fibrostenosis, stricturing and/or penetrating disease, obstructive disease, or a disease that is refractory (e.g., mrUC, refractory CD), perianal CD, or other complicated forms of IBD.

Non-limiting examples of “sample” include any material from which nucleic acids and/or proteins can be obtained. As non-limiting examples, this includes whole blood, peripheral blood, plasma, serum, saliva, mucus, urine, semen, lymph, fecal extract, cheek swab, cells or other bodily fluid or tissue, including but not limited to tissue obtained through surgical biopsy or surgical resection. In various embodiments, the sample comprises tissue from the large and/or small intestine. In various embodiments, the large intestine sample comprises the cecum, colon (the ascending colon, the transverse colon, the descending colon, and the sigmoid colon), rectum and/or the anal canal. In some embodiments, the small intestine sample comprises the duodenum, jejunum, and/or the ileum. Alternatively, a sample can be obtained through primary patient derived cell lines, or archived patient samples in the form of preserved samples, or fresh frozen samples. A sample may comprise blood, serum, plasma, sweat, hair, tears, urine, saliva, stool, or combination thereof.

The term “biomarker” comprises a measurable substance in a subject whose presence, level, or activity, is indicative of a phenomenon (e.g., phenotypic expression or activity; disease, condition, subclinical phenotype of a disease or condition, infection; or environmental stimuli). In some embodiments, a biomarker comprises a gene, gene expression product (e.g., RNA or protein), or a cell-type (e.g., immune cell).

The term “serological marker,” as used herein refers to a type of biomarker representing an antigenic response in a subject that may be detected in the serum of the subject. In some embodiments, a serological comprises an antibody against various fungal antigens. Non-limiting examples of a serological marker comprise anti-Saccharomyces cerevisiae antibody (ASCA), an anti-neutrophil cytoplasmic antibody (ANCA), E. coli outer membrane porin protein C (OmpC), anti-Malassezia restricta antibody, anti-Malassezia pachydermatis antibody, anti-Malassezia furfur antibody, anti-Malassezia globasa antibody, anti-Cladosporium albicans antibody, anti-laminaribiose antibody (ALCA), anti-chitobioside antibody (ACCA), anti-laminarin antibody, anti-chitin antibody, pANCA antibody, anit-12 antibody, and anti-Cbir1 flagellin antibody.

The term “microbiome” and its variation used herein describe the populations and interactions of the bacteria, fungi, protists, and virus that align the gastrointestinal tract of a subject. A subject afflicted with IBD may possess presence, absence, excess, diminished, or a combination thereof of a microbiome s compared to a healthy subject.

The terms “non-response,” or “loss-of-response,” as used herein, refer to phenomena in which a subject or a patient does not respond to the induction of a standard treatment (e.g., anti-TNF therapy), or experiences a loss of response to the standard treatment after a successful induction of the therapy. The induction of the standard treatment may include 1, 2, 3, 4, or 5, doses of the therapy. A “successful induction” of the therapy may be an initial therapeutic response or benefit provided by the therapy. The loss of response may be characterized by a reappearance of symptoms consistent with a flare after a successful induction of the therapy.

The section headings used herein are for organizational purposes only and are not to be construed as limiting the subject matter described.

### EXAMPLES

The following examples are included for illustrative purposes only and are not intended to limit the scope of the invention.

### Example 1: Post-Operative Prophylaxis Persistence (POPP) in Crohn's Disease

A single center, retrospective study of adult and pediatric surgically naïve ileal or ileocolonic CD subjects undergoing ileocecal and/or small bowel resection between Jan. 1, 2000-Dec. 31, 2021 and prescribed post-operative prophylaxis (5-aminosalicylate, immunomodulator, and/or biologic) was conducted. Subjects who stopped prophylaxis for non-medical reasons were excluded (e.g., self-discontinued, insurance denial). The primary outcome was time to prophylaxis failure, which was defined as requiring recurrent surgery or changing therapy due to POR despite optimized dose/drug level, immunogenicity, and/or adverse event. IBD serologies (ELISA) and genetics (Immunochip) were generated. Quartile sum scores (QSS) were calculated from IBD serologies. Cox proportional hazard analyses were adjusted for demographic and disease characteristics and principal components. Canonical pathway analysis was generated in Ingenuity Pathway Analysis. Results: Of 552 subjects, 57.6% (n=318) had prophylaxis failure.

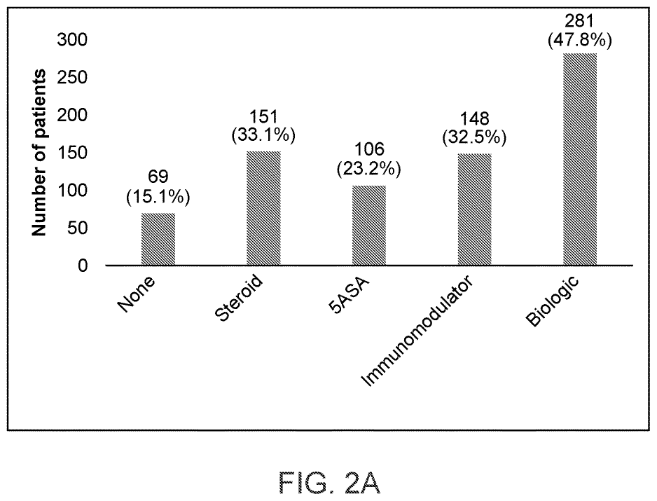

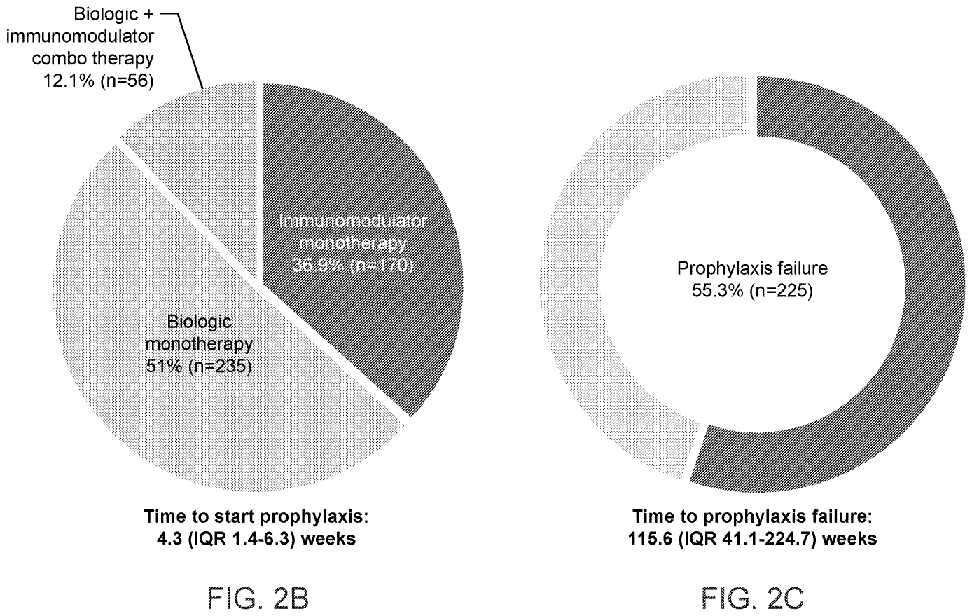

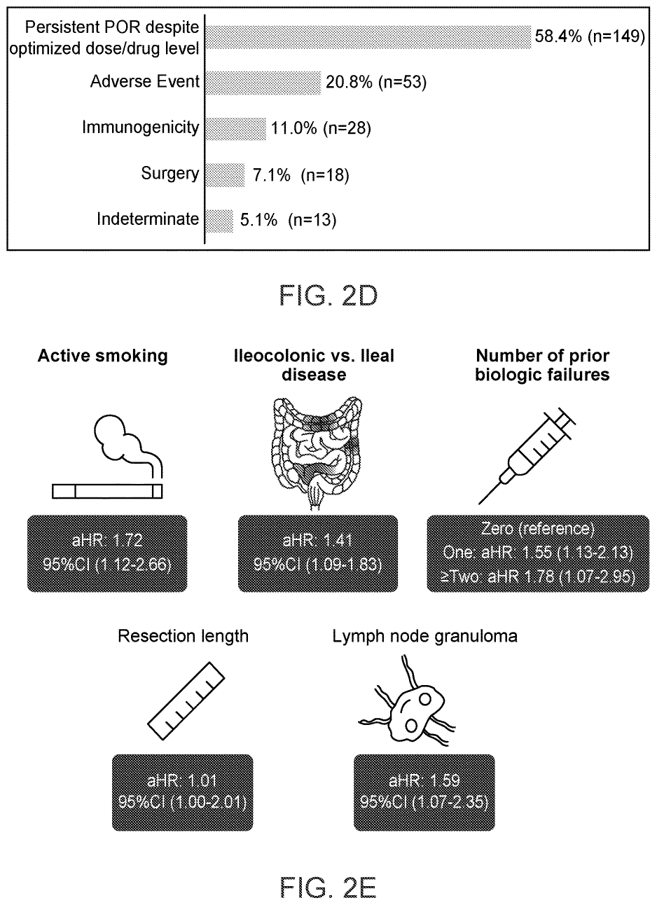

Demographics and CD characteristics are depicted in Table 1. Pre-operative medications are depicted in FIG. 2A. 72.5% (n=334) of subjects were biologic naïve or on their first biologic prior to surgery.

TABLE 1
Subject population

| | |
|---|---|
| Demographic information | Total n = 461 |

| | | |
|---|---|---|
| Median age (years, interquartile range | 28.7 | (21.9-39.4) |
| (IQR)) | | |
| Female, n(%) | 214 | (46.4) |
| European ancestry, n(%) | 419 | (91.1) |
| Median BMI at surgery (kg/m², IQR) | 22.1 | (19.0-25.9) |
| Median disease duration (months, IQR) | 67.0 | (16.4-124.) |
| Median age at diagnosis (years, IQR) | 21.7 | (16.4-31.3) |
| Active smoking at surgery, n(%) | 33 | (7.4) |
| Disease location, n(%) | | |
| Ileal (L1) | 228 | (49.6) |
| Ileocolonic (L3) | 232 | (50.4) |
| Upper GI (L4) | 46 | (10.0) |
| Disease behavior, n(%) | | |
| Non-fibrostenosing, non-penetrating (B1) | 24 | (5.2) |
| Fibrostenosing (B2) | 227 | (49.5) |
| Internal penetrating (B3) | 95 | (20.7) |
| Fibrostenosing/penetrating (B2/3) | 113 | (24.6) |
| Perianal Crohn's disease, n(%) | 103 | (22.3) |

The effects of post-operative prophylaxis is depicted in FIG. 2B and FIG. 2C. The time to start prophylaxis was 4.3 weeks (IQR 1.4-6.3 weeks). 55.3% of subjects experienced prophylaxis failure. The time to prophylaxis failure was 115.6 weeks (IQR: 41.1-224.7), as shown in FIG. 2C. The reason for prophylaxis failure is depicted In FIG. 2D. 58.4% of subjects experience persistence POR despite an optimized dose or drug level. Clinical factors associated with POPP are depicted in FIG. 2E. These include active smoking (aHR 1.72, 95% CI (1.12-2.66)), ileocolonic vs ileal disease (aHR 1.31, 95% CI (1.09-1.83)), number of prior biologic failures (One: aHR: 1.55 (1.13-2.13), ≥Two: aHR 1.78 (1.07-2.95)), resection length (aHR: 1.01, 95% CI (1.00-2.01)) and lymph node granuloma (aHR: 1.59, 95% CI (1.07-2.35)). Smoking, ileocolonic CD, greater number of prior biologics, and presence of lymph node granulomas were associated with decreased prophylaxis persistence.

Associated serological factors are listed in Table 2. Both ANCA and IgG ASCA has significant p-values.

TABLE 2
Serological factors

| Serology | aHR | P-value |
|---|---|---|
| ANCA | 1.01 | 0.02 |
| Cbir1 | 1.0005 | 0.75 |
| I2 | 1.0002 | 0.32 |
| OmpC | 1.0016 | 0.67 |
| IgA ASCA | 0.995 | 0.04 |
| IgG ASCA | 0.997 | 0.33 |

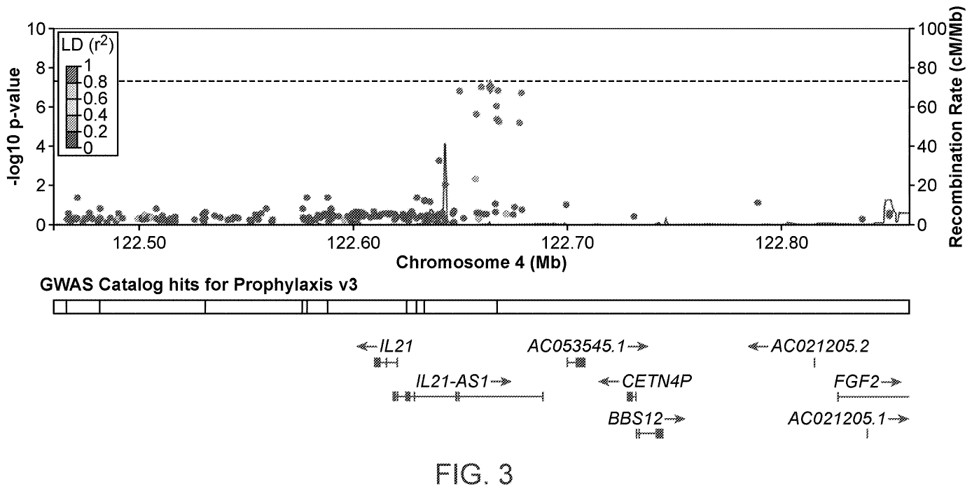

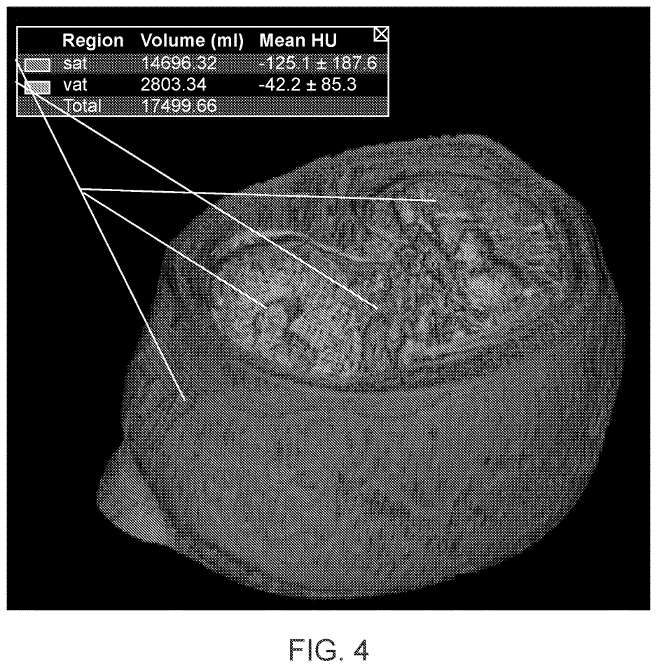

Genetic factors associated with time to prophylaxis failure are in Table 3. Loci containing IL21 met GWAS level of significant with decreased prophylaxis persistence (FIG. 3). Further, HLA DQA1*05, had a HR 0.98, p=0.88. Table 4 depicts a conditional analysis of SNPs tagging the IL21 locus.

TABLE 3
Associated Genetics Factors

| | | Minor | | | |
|---|---|---|---|---|---|
| | Chromo- | allele | | Hazard | |
| SNP | some | frequency | Gene | ratio | P-value |
| rs6811007 | 4 | 0.22 | IL21 \| BBS12 | 1.89 | 4.66e−8 |
| rs6874652 | 5 | 0.13 | MSH3 | 1.91 | 1.01e−5 |
| rs1882083 | 7 | 0.19 | ELMO1 | 0.53 | 2.41E−5 |
| rs7823671 | 8 | 0.12 | LRP12 | 2.00 | 3.01e−5 |

TABLE 4
conditional analysis of SNPs tagging the IL21 locus

| | | | | Minor | | | p-value after |
|---|---|---|---|---|---|---|---|
| | | | | allele | Hazards | | conditional |
| SNP | Position | A0 | A1 | frequency | ratio | p-value | analysis |
| rs13132308 | 123551114 | G | A | 0.10 | 1.43 | 0.04 | 0.106 |
| rs28517551 | 123561459 | A | G | 0.08 | 1.90 | 5.20E−04 | 0.767 |
| rs1533236 | 123564458 | A | G | 0.47 | 1.32 | 0.01 | 5.14E−03 |
| rs7678116 | 123571118 | A | G | 0.21 | 1.88 | 1.48E−07 | 0.443 |
| rs12511287 | 123578531 | T | A | 0.42 | 1.38 | 4.51E−03 | 0.559 |
| rs4833253 | 123578850 | G | A | 0.23 | 1.75 | 2.28E−06 | 0.401 |
| rs6844999 | 123581235 | C | A | 0.22 | 1.90 | 9.25E−08 | 1.000 |
| rs6825988 | 123582081 | A | G | 0.45 | 1.37 | 0.02 | 0.307 |
| rs4833841 | 123584597 | A | G | 0.21 | 1.87 | 1.94E−06 | 0.895 |
| rs6811007 | 123585362 | A | G | 0.22 | 1.90 | 9.25E−08 | 1.000 |
| rs9685849 | 123588318 | C | G | 0.23 | 1.86 | 8.68E−07 | 0.201 |
| rs925550 | 123588526 | A | C | 0.23 | 1.72 | 4.12E−06 | 0.165 |
| rs6837455 | 123588942 | C | G | 0.22 | 1.89 | 1.41E−07 | 0.994 |
| rs6534357 | 123589353 | A | T | 0.23 | 1.71 | 5.38E−06 | 0.106 |
| rs1398559 | 123593320 | G | A | 0.34 | 1.46 | 1.76E−03 | 0.872 |
| rs12501008 | 123597440 | A | G | 0.23 | 2.12 | 8.83E−06 | 0.507 |
| rs7664318 | 123599124 | G | A | 0.23 | 1.69 | 6.14E−06 | 0.124 |
| rs75802552 | 123599326 | D | I | 0.36 | 1.41 | 0.01 | 0.121 |
| rs60318098 | 123599898 | G | A | 0.22 | 1.86 | 1.86E−07 | 0.736 |

A transcriptomics binding site analysis was conducted for rs6811007. The SNP intersects TF binding sites from chip-seq data for ATF3, CEBPG, SMAD4, ATF4, ZBTB40, ETV1, ZEB2, LEF1, DDIT3, HDAC1, IKZF1, POLR2H, CEBPB, ZNF589, TRIM24, ATF7, ELF1 and CXXC5 in blood, as well as CTCF specifically in activated CD4-positive, alpha-beta T cells. Chromatin state data from chromHMM showed the SNP fell within active enhancer region in connective tissue mesenchymal stem cells and Th1 cells, is in flanking TSS downstream sites in naive thymus-derived CD4-positive, alpha-beta T cells and CD4-positive, alpha-beta memory T cells and in flanking TSS upstream sites in CD4-positive, alpha-beta T cells and Th17 cells. Unfortunately, it's quiescent/low or a weak enhancer in GI related tissues (colon, small intestine).

DNAse or ATAC accessibility experiments showed rs6811007 falls in open chromatin sites in activated T cells (CD4+, CD8+ and Th17). rs6811007 did not overlap any transcription factor motifs.

### Example 2: Pre-Operative Visceral Adipose Tissue (VAT) Quality but not Quantity was Associated with Post-Operative Recurrence (POR) in Crohn's Disease

Methods: A single center, retrospective study of adult and pediatric ileal and ileocolonic CD subjects undergoing ileocecal or small bowel resection between Jan. 1, 2007 to Dec. 31, 2021 was conducted. Subjects with pre-operative CT or MRI abdomen/pelvis <12 months of surgery and a colonoscopy <15 months after surgery were included. 3D measurements of VAT volume (cm) and ratio of VAT to subcutaneous adipose tissue (SAT) volume (VAT:SAT) were obtained from CT and MRI. VAT radiodensity (HU) and ratio of VAT:SAT radiodensity were obtained from CT. A representative image is depicted in FIG. 4. Of note, the radiodensity of adipose tissue is −150 to −50 HU, and lower adipose radiodensity is suggested to reflect poorer fat quality. The primary outcome was early endoscopic POR (ePOR) defined as Rutgeert's score>i2 on colonoscopy <15 months after surgery. The secondary outcome was ePOR severity (Rutgeert's score i0-4). To assess the association between the VAT metrics and early ePOR and ePOR severity, binomial and ordinal logistic regressions were performed, respectively, adjusting for demographic and disease related variables to calculate adjusted odds ratio (aOR) and 95% confidence interval(CI). Results: Of the 233 subjects included, 43% of subjects developed early ePOR (n=99). Table 5 summarizes cohort demographics. The median time between imaging and surgery was 1.52 months (interquartile range 0.49-3.50), and 58% (n=134) underwent CT. VAT volume (aOR 1.233 95% CI [0.857-1.746]) and VAT:SAT volume (aOR 0.849 95% CI [0.610-1.818], Table 6) were not associated with early ePOR. VAT radiodensity (aOR 0.631, 95% CI [0.430-0.926]) and VAT:SAT radiodensity (aOR 5.559 95% CI [1.184-26.094]) were significantly associated with early ePOR. VAT volume (aOR 1.142 95% CI [0.854-1.528]) and VAT:SAT volume (aOR 0.896 95% CI [0.680-1.773] were not associated with ePOR severity. VAT radiodensity (aOR 0.633, 95% CI [0.458-0.874]) and VAT:SAT radiodensity (aOR 4.160 95% CI [1.107-15.630]) were significantly associated with ePOR severity.

TABLE 5
Baseline cohort characteristics

| | |
|---|---|
| Variables | Total (n = 233) |

| | | |
|---|---|---|
| Mean age (years, SD) | 37.66 | (17.91) |
| Female, n(%) | 111 | (47.06) |
| European ancestry, n(%) | 213 | (90.25) |
| Mean BMI (kg/m², SD) | 22.67 | (4.92) |
| Smoking status, n(%) | 11 | (4.78) |
| Mean age at diagnosis (years, SD) | 25.58 | (15.03) |
| Mean disease duration (months, SD) | 145.22 | (141.08) |

CD Disease location, n(%)

| | | |
|---|---|---|
| Ileal (L1) | 110 | (46.22) |
| Ileocolonic (L3) | 128 | (53.78) |
| Upper GI (L4) | 19 | (7.98) |

CD Behavior, n(%)

| | | |
|---|---|---|
| Non-fibrostenosing, non-penetrating (B1) | 18 | (7.59) |
| Fibrostenosing (B2) | 108 | (45.57) |
| Internal penetrating (B3) | 48 | (20.25) |
| Fibrostenosis & internal penetrating (B2/3) | 63 | (26.58) |
| Perianal CD, n(%) | 43 | (18.07) |

Pre-operative medication, n(%)

| None | 51 | (21.52) |
|---|---|---|
| Steroid | 66 | (27.85) |
| 5-aminosalicylate | 28 | (11.81) |
| Immunomodulator | 57 | (24.05) |
| Biologic | 139 | (58.65) |
| Post-operative prophylaxis, | n(%) 198 (83.19) | |
| Antibiotics | 3 | (1.52) |
| 5-aminosalicylate | 12 | (6.06) |
| Immunomodulator | 38 | (19.19) |
| Biologic | 169 | (85.35) |
| Prior surgery, n(%) | 59 | (24.79) |
| Pre-operative imaging: CT, n(%) | 134 | (56.54) |

TABLE 6
VAT radiodensity and volumetrics

| | Early ePOR | Severity of ePOR |
|---|---|---|
| VAT Metric | aOR (95% CI)* | aOR (95% CI)* |
| VAT Volume (cm³) | 1.233 (0.857-1.746) | 1.142 (0.854-1.528) |
| VAT:SAT Volume | 0.849 (0.610-1.818) | 0.896 (0.680-1.773) |
| VAT Radiodensity (HU) | 0.631 (0.430-0.926) | 0.633 (0.458-0.874) |
| VAT:SAT Radiodensity | 5.559 (1.184-26.094) | 4.160 (1.107-15.630) |

VAT: visceral adipose tissue,
SAT: subcutaneous adipose tissue,
aOR—adjusted odds ratio,
CI—confidence interval,
HU—Houndsfield unit
*Adjusted for age, sex, smoking status, prior surgery, internal penetrating disease behavior, and post-operative prophylaxis.

Lower radiodensity of VAT was independently associated with increased risk of early ePOR and more severe ePOR.

Conclusion: VAT radiodensity metrics are associated with early ePOR and ePOR severity in this cohort. These findings suggest VAT quality may be a novel prognostic marker for POR and provide insight into the underlying biology of POR.

### Example 3: Genetic Variation in Hepatic Fibrosis and Cytokine Signaling Pathways Enriched in CD Cases with Post-Operative Recurrence Pre- and During the Biological Era

Methods: We conducted a retrospective study in two cohorts of adult and pediatric surgically naïve CD subjects undergoing ileocecal and/or small bowel resection between Jan. 1, 1950-12/31/2021 followed at a quaternary IBD center. Cohort 1 (pre-biologic era) and Cohort 2 (biologic era) included subjects undergoing surgeries before and after Jan. 1, 1999, respectively. Differences between cohorts were evaluated with Mann-Whitney U and X² testing for continuous and categorical variables, respectively. Primary outcome was time to second surgery. We performed Cox proportional hazard analyses adjusting for gender, smoking, and principal components for each cohort. Conical pathway analysis was performed (Ingenuity Pathway Analysis). We performed a meta-analysis of genetic and conical pathway associations with both cohorts.

Results: Cohort 1 had 284 subjects, and Cohort 2 had 852 subjects. Table 7 summarizes differences between the cohort. In Cohort 1, I2 titer was associated with surgical POR (adjusted hazards ratio (aHR) 1.008, 95% confidence interval(CI) [1.003-1.013]). In Cohort 2, IgA ASCA titer was protective of surgical POR (aHR0.990, 95% CI [0.985-0.999]). In the meta-analysis no SNPs achieved genome-wide significance. Table 8 summarizes enriched pathways associated with POR. In Cohort 1, canonical pathway analysis yielded enriched pathways involving cytokine storm signaling and hepatic fibrosis signaling. In cohort 2, canonical pathway analysis yielded enriched pathways involving STAT3, pathogen induced cytokine storm signaling, Th1 and Th2 activation signaling, and hepatic fibrosis signaling. In pathway meta-analysis, enriched pathways included pathogen induced cytokine storm signaling, Th1 and Th2 activation, IL-10, and hepatic fibrosis signaling.

TABLE 7
Clinical differences in CD patients undergoing surgery during pre-vs biologic era

| | Pre-biologic | | Biologic era | | |
|---|---|---|---|---|---|
| | era (n = 284) | | (n = 852) | | p-value |
| Mean age at surgery (years, SD) | 26.77 | (10.61) | 33.02 | (15.62) | 2.57e⁻⁷ |
| Female, n(%) | 127 | (44.72) | 406 | (47.65) | 0.391 |
| Caucasian, n(%) | 270 | (95.07) | 773 | (91.16) | 0.034 |
| Active smoking at surgery, n(%) | 42 | (19.91) | 72 | (8.80) | 5.00e⁻⁶ |
| Median disease duration at surgery | 42.55 | (0.79-42.55) | 60.87 | (14.00-123.87) | 1.30e⁻⁵ |
| (months, IQR) | | | | | |
| CD Location, n(%) | | | | | 0.931 |
| Ileal (L1) | 150 | (53.00) | 452 | (53.30) | |
| Ileocolonic (L3) | 133 | (47.00) | 396 | (46.70) | |
| Upper GI (L4) | 9 | (3.17) | 74 | (8.69) | 0.002 |
| CD behavior, n(%) | | | | | 3.20e⁻⁵ |
| Non-fibrostenosing, non-penetrating | 46 | (16.61) | 64 | (7.53) | |
| (B1) | | | | | |
| Fibrostenosing (B2) | 121 | (43.68) | 423 | (49.76) | |
| Internal Penetrating (B3) | 67 | (24.19) | 182 | (21.41) | |
| Fibrostenosing/Penetrating (B2/3) | 43 | (15.52) | 181 | (21.29) | |
| Perianal CD, n(%) | 63 | (22.18) | 168 | (19.72) | 0.371 |
| Indication for surgery, n(%) | | | | | 1.20e⁻⁵ |
| Medically refractory disease | 52 | (20.31) | 82 | (9.84) | |
| Stricture/obstruction | 116 | (45.31) | 477 | (57.26) | |
| Penetrating complication | 88 | (34.38) | 274 | (32.89) | |
| Median length of resection (cm, IQR) | 30.48 | (21.00-60.96) | 22.00 | (14.35-34.00) | 5.18e⁻⁹ |
| Post-operative prophylaxis, n(%) | 89 | (43.63) | 641 | (77.89) | 4.41e⁻²² |
| Early endoscopic exam ≤15 months | 10 | (13.70) | 514 | (75.15) | 3.03e⁻²⁷ |
| post-op, n(%) | | | | | |
| Recurrent surgery, n(%) | 187 | (69.37) | 154 | (18.08) | 5.02e⁻⁵⁹ |
| Median time to second surgery | 156.63 | (96.00-238.37) | 91.60 | (44.18-130.63) | 5.60e⁻¹⁴ |
| (months, IQR) | | | | | |

SD: standard deviation,
IQR: interquartile range

TABLE 8
Enriched pathways for surgical POR

| | |
|---|---|
| Cohort 1 | Cytokine storm signaling (p = 2.51e−13) |
| | Hepatic fibrosis signaling (p = 7.94e−11) |
| Cohort 2 | STAT3 (p = 6.31e−12) |
| | Pathogen induced cytokine storm signaling |
| | (p = 5.25e−9) |
| | Th1 and Th2 activation signaling (p = 1.51e−8) |
| | Hepatic fibrosis signaling (p = 6.61e−8) |
| Meta | Pathogen induced cytokine storm signaling |
| analysis | (p = 2.00e−15) |
| | Th1 and Th2 activation (p = 3.24e−10) |
| | IL-10 (p = 1.47e−9) |
| | Hepatic fibrosis signaling (p = 2.82e−9). |

Conclusions: Meta-analyses of two cohorts of surgical CD patients from pre- and post-surgical demonstrated genetic variation enriched for pathways in pathogen induced cytokine storm signaling, Th1 and Th2 activation, and fibrosis were associated with POR. Cytokine storm signaling and hepatic fibrosis signaling pathways were significantly associated with POR in both biologic era and pre-biologic era cohorts.

### Example 4: Comparative Persistence of Tumor Necrosis Factor Antagonists (TNFI) Vs. Non-TNFI for Post-Operative Prophylaxis in Crohn's Disease

Methods: A single center, retrospective study of adult and pediatric ileal or ileocolonic CD subjects undergoing their first ileocecal and/or small bowel resection between Jan. 1, 2000-12/31/2021 and prescribed a biologic (infliximab, adalimumab, certolizumab, anti-integrin, or anti-IL12/23) for post-operative prophylaxis was conducted. Subjects who stopped prophylaxis for non-medical reasons were excluded (e.g., self-discontinued, insurance denial). Subjects were grouped as being on TNFi or non-TNFi therapy. Differences between treatment groups were evaluated with Mann-Whitney U and X² for continuous and categorical variables, respectively. The primary outcome was time to prophylaxis failure, which was defined as requiring recurrent surgery or change in therapy due to POR despite optimized dose/drug level, immunogenicity, and/or adverse event. The secondary outcome was early ePOR, which was defined as Rutgeert's score >12 on endoscopic exam <15 months after surgery. Risk of prophylaxis failure and early ePOR were evaluated with Cox proportional hazard and logistic regression, respectively, adjusting for demographic and disease characteristics.

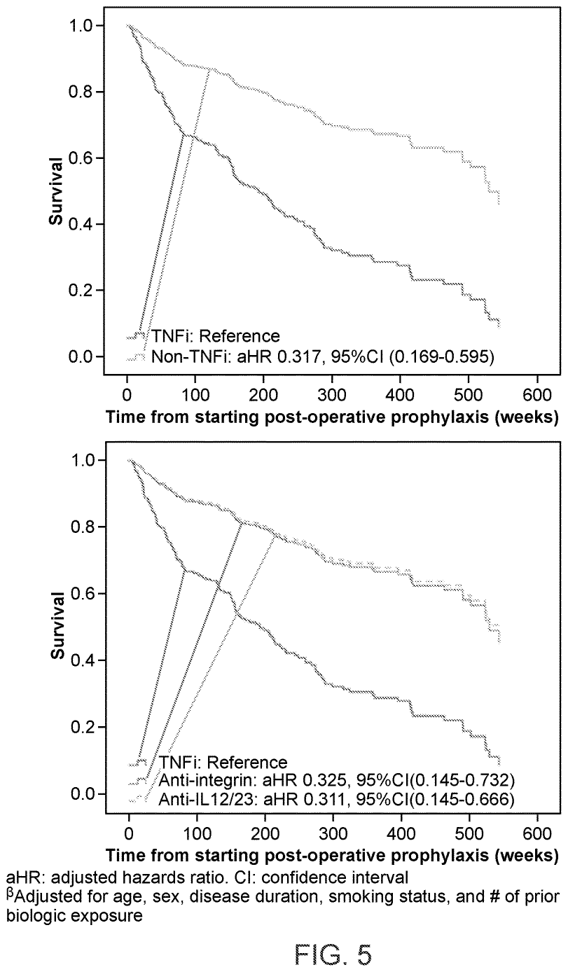

Results: A total of 316 subjects were included (TNFi 81%). Median follow-up time was 79.6 months. Table 9 summarizes group differences. TNFi prophylaxis had numerically higher rates of failure due to immunogenicity (21% vs. 11%, p=0.31) and adverse events (26% vs.11%, p=0.16). After multivariable adjustment, non-TNFi prophylaxis was associated with a significantly lower risk of prophylaxis failure (adjusted hazard ratio 0.317, 95% confidence interval(CI) [0.169-0.595], FIG. 5) than TNFi. Non-TNFi prophylaxis had similar risk of early ePOR as TNFi (adjusted odds ratio 0.789, 95% CI [0.364-1.709]). Stratifying non-TNFi by drug class (anti-integrin and anti-IL 12/23) yielded similar results (FIG. 5).

TABLE 9
Differences between post-operative prophylaxis with TNFi vs non-TNFi

| | TNFi | | Non-TNFi | | |
|---|---|---|---|---|---|
| Variable | (n = 256) | | (n = 60) | | p-value |
| Median age (years, interquartile range (IQR)) | 27.3 | (20.9-37.7) | 27.3 | (19.4-44.5) | 0.847 |
| Female, n(%) | 129 | (50.4) | 27 | (45.0) | 0.452 |
| Caucasian, n(%) | 225 | (87.9) | 57 | (95.0) | 0.110 |
| Median BMI at surgery (kg/m², IQR) | 21.8 | (18.6-26.0) | 21.8 | (17.9-24.2) | 0.325 |
| Median disease duration (months, IQR) | 60.9 | (15.5-28.5) | 104.7 | (45.5-167.8) | 0.000213 |
| Median age at diagnosis (years, IQR) | 20.7 | (15.4-28.5) | 19.2 | (11.6-31.9) | 0.156 |
| Active smoking at surgery, n(%) | 23 | (9.3) | 0 | | 0.015 |
| Crohn's disease location, n(%) | | | | | |
| Ileal (L1) | 117 | (45.7) | 28 | (46.7) | 0.893 |
| Ileocolonic (L3) | 139 | (54.3) | 32 | (53.3) | 0.893 |
| Upper GI (L4) | 28 | (10.9) | 10 | (16.7) | 0.219 |
| Crohn's disease behavior, n(%) | | | | | |
| Non-fibrostenosing, non-penetrating (B1) | 20 | (7.8) | 3 | (5.0) | 0.450 |
| Fibrostenosing (B2) | 123 | (48.2) | 33 | (55.0) | 0.332 |
| Internal penetrating (B3) | 46 | (18.0) | 10 | (16.7) | 0.812 |
| Fibrostenosing/penetrating (B2/3) | 66 | (25.9) | 14 | (23.3) | 0.695 |
| Perianal Crohn's disease, n(%) | 50 | (19.5) | 17 | (28.3) | 0.133 |
| # of prior biologic exposures | | | | | |
| 0 | 194 | (75.8) | 12 | (20.0) | 3.244e⁻¹⁴ |
| 1 | 54 | (21.1) | 22 | (36.7) | 0.011 |
| ≥2 | 8 | (3.1) | 26 | (43.3) | 1.473e⁻¹⁹ |
| Prophylaxis: Concomitant immunomodulator | 55 | (21.5) | 5 | (8.3) | 0.019 |
| Surgical resection histopathology | | | | | |
| Median length of small bowel resected (cm, IQR) | 22.6 | (14.5-33.5) | 19.3 | (11.1-30.8) | 0.140 |
| Clean resection margins, n(%) | 168 | (85.7) | 47 | (87.0) | 0.804 |
| Lymph node granuloma, n(%) | 33 | (16.9) | 12 | (21.8) | 0.404 |
| Time to start prophylaxis after surgery (weeks) | 4.4 | (3.7-7.9) | 4.7 | (3.1-8.6) | 0.850 |
| Early endoscopic POR, n(%) | 65 | (36.7) | 16 | (31.4) | 0.482 |
| Prophylaxis failure, n(%) | 142 | (55.5) | 18 | (30.0) | 0.000383 |
| Reason for prophylaxis failure, n(%) | | | | | |
| Indeterminate^{α} | 12 | (8.5) | 0 | | 0.198 |
| Failure despite optimized dose/drug level | 56 | (39.4) | 11 | (61.1) | 0.079 |
| Immunogenicity | 30 | (21.1) | 2 | (11.1) | 0.317 |
| Adverse event | 37 | (26.1) | 2 | (11.1) | 0.164 |
| Recurrent surgery | 12 | (8.5) | 3 | (16.7) | 0.260 |

^{α}Unable to discern between failure due to optimized therapy vs. immunogenicity because attenuation of symptoms could indicate either

Conclusion: In a cohort of surgically naïve CD subjects prescribed a biologic for post-operative prophylaxis, non-TNFi prophylaxis had significantly greater persistence than TNFi while having similar risk for early ePOR.

### Example 5: Comparative Persistence of Tumor Necrosis Factor Antagonists (TNFI) Vs. Non-TNFI for Post-Operative Prophylaxis in Crohn's Disease

A further analysis of the retrospective study conducted in Example 4 was performed, which refined the relevant subjects considered in the study, and which provides further data on the subjects analyzed.

Methods: A single center, retrospective study of adult and pediatric ileal or ileocolonic CD subjects undergoing their first ileocecal and/or small bowel resection between Jan. 1, 2000-12/31/2021 and prescribed a biologic (infliximab, adalimumab, certolizumab, anti-integrin, or anti-IL12/23) for post-operative prophylaxis was conducted. Subjects who stopped prophylaxis for non-medical reasons were excluded (e.g., self-discontinued, insurance denial). Subjects were grouped as being on an inhibitor of TNF (TNFi) (e.g., infliximab, adalimumab, certolizumab) or non-TNFi therapy (e.g., anti-integrin, or anti-IL12/23). Timing of post-operative prophylaxis was defined by when a subject received the first dose based on provider documentation.

The risk of prophylaxis failure (requiring recurrent surgery or discontinuation of therapy due to persistent POR despite optimized drug level or dose escalation, immunogenicity, and/or adverse event) and early endoscopic POR (Rutgeert's score >12 within 15 months post-operatively) between non-TNF and TNF antagonist prophylaxis using Cox proportional hazard and logistic regression, respectively, adjusting for demographic and disease characteristics.

The primary outcome was time to prophylaxis failure, which was defined as requiring recurrent surgery or change in therapy due to POR despite optimized dose/drug level, immunogenicity, and/or adverse event. The secondary outcome was early ePOR, which was defined as Rutgeert's score >12 on endoscopic exam<15 months after surgery. Risk of prophylaxis failure and early ePOR were evaluated with Cox proportional hazard and logistic regression, respectively, adjusting for demographic and disease characteristics. Endoscopy reports were reviewed, and if a Rutgeert's score was not recorded, text and imaging were retrospectively applied by a trained IBDologist (PG).

Differences between treatment groups were evaluated with Mann-Whitney U and X² for continuous and categorical variables, respectively. To evaluate the comparative persistence of TNF and non-TNF antagonists, we performed a survival analysis using multivariable Cox proportional hazard regression adjusting for age, sex, CD disease duration, pre-operative smoking status, pre-operative biologic mechanism and number of prior biologic exposures, which were determined a priori. To evaluate the comparative risk for early endoscopic POR between TNF and non-TNF antagonists, a multivariable logistic regression, adjusting for age, sex, CD disease duration, pre-operative smoking status, and internal penetrating disease behavior, which were determined a priori. Stratified analyses comparing TNF antagonists to anti-integrins and anti-IL12/23, respectively, were performed for both outcomes.

Results: A total of 291 subjects were included (TNFi 81%). Median follow-up time was 55.7 months, and the median time to start post-operative prophylaxis was 4.4 weeks. Table 10 summarizes group differences.

Compared to TNF antagonist prophylaxis, subjects prescribed a non-TNF antagonist for prophylaxis had longer disease duration (p=0.001), were less likely to be active smokers (p=0.015), less likely biologic naive (p=3.02e-13), and less likely to be prescribed a concomitant immunomodulator (p=0.034). Also, subjects prescribed non-TNF antagonist prophylaxis were more likely to use non-TNF antagonists pre-operatively (p=2.61e-30).

On follow-up, 50.2% (n=146) subjects experienced prophylaxis failure with median time to prophylaxis failure of 88.9 weeks. The most common reason for prophylaxis failure was persistent POR despite optimized drug concentration level or dose escalation (43.8%, n=64) followed by adverse event (24.7%, n-36) and immunogenicity (18.5%, n=27). On multivariable Cox proportional hazard regression, subjects prescribed non-TNF antagonist prophylaxis were significantly less likely to experience prophylaxis failure than those prescribed TNF antagonists (adjusted hazard ratio (aHR) 0.26; 95% confidence interval(CI) [0.13-0.53]. When stratifying non-TNF antagonists by drug mechanism, subjects prescribed anti-integrins (aHR 0.32; 95% CI [0.14-0.76]) and anti-IL12/23 (aHR 0.21; 95% CI [0.09-0.51]) for prophylaxis were significantly less likely to experience prophylaxis failure compared to those prescribed a TNF antagonist. Sensitivity analyses after excluding subjects who underwent surgery before 2014 and after excluding subjects who did not undergo colonoscopy within 15 months of surgery yield similar results. When comparing reasons for prophylaxis failure, TNF antagonist prophylaxis had nominally higher rates of adverse events (26% vs.12%, p=0.232) compared to non-TNF antagonist prophylaxis.

After their index surgery, 85.3% (n=214) subjects had an endoscopic exam within 15 months, and 35.2% (n=74) of these subjects developed early endoscopic POR. Of those with early endoscopic POR, 24.8% (n=52) had Rutgeert's score i2, 5.2% (n=11) i3, and 5.2% (n=11) 14. On multivariable logistic regression, non-TNF antagonist prophylaxis had similar risk for early endoscopic POR as TNF antagonist prophylaxis (adjusted odds ratio (aOR) 0.66; 95% CI [0.32-1.36]. When stratifying non-TNF antagonists by drug mechanism, prophylaxis with anti-integrins (aOR 0.71; 95% CI [0.21-2.39]) and anti-IL12/23 (aOR 0.64; 95% CI [0.27-1.49]) had similar risk for early endoscopic POR as TNF antagonists.

In the overall cohort, on multivariable Cox proportional hazard regression, pre-operative active smoking (aHR 1.98; 95% CI [1.14-3.43]), number of prior biologic exposures (1 prior biologic: aHR 1.85; 95% CI [1.24-2.77]; >2 prior biologics: aHR 1.98; 95% CI [1.10-3.56]), ileocolonic CD (aHR 1.57; 95% CI [1.11-2.22]), and longer resection length (aHR 1.02; 95% CI [1.01-1.02]) were associated with increased risk of prophylaxis failure. In the overall cohort, on multivariable logistic regression, European ancestry was associated with risk of early endoscopic POR (aOR 5.78; 95% CI [1.28-26.21]).

This single-center cohort of surgically naïve CD subjects undergoing ICR and/or SBR who were prescribed a biologic for post-operative prophylaxis, found non-TNF antagonists may be more favorable long-term prophylactic therapies than TNF-antagonists with similar efficacy for preventing early endoscopic POR.

Conclusion: In a cohort of surgically naïve CD subjects prescribed a biologic for post-operative prophylaxis, non-TNF antagonists had greater persistence than TNF antagonists with similar risk for early endoscopic POR. If confirmed by large, prospective studies, these findings can inform post-operative management strategies in CD.

TABLE 10
Differences between post-operative prophylaxis with TNFi vs non-TNFi

| | | | Non-TNF | | |
|---|---|---|---|---|---|
| | TNF antagonist | | antagonist | | p- |
| Variable | (n = 236) | | (n = 55) | | value |
| Median age (years, interquartile | 28.5 | (22.3-38.6) | 33.0 | (21.1-46.3) | 0.755 |
| range (IQR)) | | | | | |
| Female, n(%) | 119 | (50.4) | 25 | (45.5) | 0.507 |
| European ancestry, n(%) | 206 | (87.3) | 52 | (94.5) | 0.126 |
| Median BMI at surgery (kg/m², | 22.3 | (19.0-26.1) | 22.1 | (18.4-24.3) | 0.343 |
| IQR) | Missing n = 68 | | Missing n = 4 | | |
| Median disease duration | 65.3 | (18.3-121.1) | 106.6 | (58.1-168.0) | 0.001 |
| (months, IQR) | Missing n = 4 | | Missing n = 0 | | |
| Median age at diagnosis (years, | 21.8 | (16.4-29.2) | 20.7 | (12.8-32.5) | 0.225 |
| IQR) | Missing n = 4 | | Missing n = 0 | | |
| Active smoking at surgery, n(%) | 23 | (10.1) | 0 | | 0.015 |
| | Missing n = 8 | | Missing n = 1 | | |
| Crohn's disease location, n(%) | | | | | |
| Ileal (L1) | 107 | (45.3) | 27 | (49.1) | 0.615 |
| Ileocolonic (L3) | 129 | (54.7) | 28 | (50.9) | 0.615 |
| Upper GI (L4) | 25 | (10.6) | 8 | (14.5) | 0.405 |
| Crohn's disease behavior, n(%) | | | | | |
| Non-fibrostenosing, non- | 18 | (7.7) | 2 | (3.6) | 0.292 |
| penetrating (B1) | | | | | |
| Fibrostenosing (B2) | 112 | (47.7) | 30 | (54.5) | 0.344 |
| Internal penetrating (B3) | 41 | (17.4) | 10 | (18.2) | 0.887 |
| Fibrostenosing/penetrating (B2/3) | 64 | (22.2) | 13 | (23.6) | 0.598 |
| Perianal Crohn's disease, n(%) | 48 | (20.3) | 16 | (29.1) | 0.158 |
| # of biologic exposures prior to | | | | | |
| immediate pre-operative biologic | | | | | |
| agent, n(%)* | | | | | |
| 0 | 175 | (74.2) | 12 | (21.8) | 3.02e⁻¹³ |
| 1 | 53 | (22.5) | 19 | (34.5) | 0.061 |
| ≥2 | 8 | (3.4) | 24 | (43.6) | 8.56e⁻¹⁸ |
| Pre-operative biologic | | | | | 2.61e⁻³⁰ |
| mechanism, n(%) | | | | | |
| None | 81 | (34.5) | 9 | (16.4) | |
| TNF antagonist | 152 | (64.7) | 15 | (27.3) | |
| Non-TNF antagonist | 2 | (0.9) | 31 | (56.4) | |
| Prophylaxis: Concomitant | 51 | (21.6) | 5 | (9.1) | 0.034 |
| immunomodulator | | | | | |
| Surgical resection histopathology | | | | | |
| Median length of small bowel | 23.5 | (15.2-35.3) | 20.1 | (10.7-33.0) | 0.086 |
| resected (cm, IQR) | Missing n = 26 | | Missing n = 2 | | |
| Histologically clean resection | 153 | (85.5) | 43 | (87.8) | 0.684 |
| margins, n(%) | Missing n = 57 | | Missing n = 12 | | |
| Lymph node granuloma, n(%) | 30 | (16.9) | 10 | (20.0) | 0.605 |
| | Missing n = 58 | | Missing n = 13 | | |
| Median time to start prophylaxis | 4.4 | (3.5-7.9) | 4.7 | (2.9-8.6) | 0.922 |
| post-operatively (weeks, IQR) | | | | | |
| Early endoscopic POR, n(%) | 61 | (37.2) | 14 | (29.8) | 0.350 |
| | Missing n = 72 | | Missing n = 8 | | |
| Prophylaxis failure, n(%) | 130 | (55.1) | 16 | (29.1) | 0.001 |
| Reason for prophylaxis failure, | | | | | |
| n(%) | | | | | |
| Indeterminate | 12 | (9.3) | 0 | | 0.203 |
| Failure despite optimized | 55 | (42.3) | 9 | (56.3) | 0.289 |
| dose/drug level | | | | | |
| Immunogenicity | 25 | (19.2) | 2 | (12.5) | 0.317 |
| Adverse event | 34 | (26.2) | 2 | (12.5) | 0.232 |
| Recurrent surgery | 9 | (6.9) | 3 | (18.8) | 0.104 |

*Biologic agents used immediately prior to surgery were not counted due to difficulty ascertaining if subjects had true non-response or disease too advanced to respond to medical therapy, and biologics used immediately pre-operatively are often resumed post-operatively.

TABLE 11
Sensitivity analyses excluding subjects who underwent surgery
before 2014 and excluding subjects who did not undergo
colonoscopy within 15 months post-operatively. TNF antagonists
is the reference group in these analyses.

| | Analysis excluding those | Analysis excluding those |
|---|---|---|
| | undergoing surgery before | without early |
| | 2014 | colonoscopy ≤15 months |
| | aHR (95% CI) | of surgery aHR (95% CI) |
| Non-TNF | 0.24 (0.11-0.56), p = 0.001 | 0.24 (0.11-0.53), p = 4.09e⁻⁴ |
| Antagonist | | |
| Anti-Integrin | 0.33 (0.12-0.89), p = 0.029 | 0.31 (0.12-0.83), p = 0.019 |
| Anti-IL12/23 | 0.19 (0.07-0.52), p = 0.001 | 0.19 (0.07-0.51), p = 0.001 |

TABLE 12
Multivariable Cox proportional hazard and logistic regression evaluating risk factors
associated with time to prophylaxis failure and early endoscopic POR, respectively,
in CD subjects prescribed biologics for post-operative prophylaxis.

| | | | Early endoscopic | |
|---|---|---|---|---|
| | Prophylaxis failure | | POR | |
| Risk factor | aHR^{δ} (95% CI) | | aOR^{†} (95% CI) | |
| Age at surgery | 1.01 | (0.99-1.02) | 1.01 | (0.99-1.00) |
| Male vs. Female | 0.93 | (0.66-1.30) | 1.56 | (0.86-2.81) |
| European ancestry vs. non- | 1.68 | (0.90-3.10) | 5.78 | (1.28-26.21) |
| European ancestry | | | | |
| BMI | 1.03 | (0.99-1.08) | 1.03 | (0.96-1.11) |
| Actively smoking at surgery | 1.98 | (1.14-3.43) | 0.69 | (0.20-2.42) |
| Disease duration | 1.00 | (0.99-1.001) | 1.000 | (0.99-1.00) |
| Crohn's disease location | | | | |
| Ileocolonic vs. ileal | 1.57 | (1.11-2.22) | 1.21 | (0.66-2.21) |
| Upper GI | 0.88 | (0.50-1.56) | 1.39 | (0.55-3.52) |
| Crohn's disease behavior | | | | |
| Non-fibrostenosing, non-penetrating | Reference | | | |
| (B1) | | | | |
| Fibrostenosing (B2) | 0.90 | (0.50-1.64) | 0.49 | (0.12-1.46) |
| Internal penetrating (B3) | 1.03 | (0.52-2.03) | 0.49 | (0.14-1.70) |
| Fibrostenosing/penetrating (B2/3) | 0.56 | (0.28-1.10) | 0.46 | (0.15-1.44) |
| Perianal disease | 0.93 | (0.62-1.39) | 0.64 | (0.30-1.35) |
| Number of prior biologic exposure | | | | |
| 0 | Reference | | | |
| 1 | 1.85 | (1.24-2.77) | 1.26 | (0.63-2.51) |
| ≥2 | 1.98 | (1.10-3.56) | 2.27 | (0.93-5.53) |
| Pre-operative biologic mechanism, | | | | |
| n(%) | | | | |
| None | Reference | | | |
| TNF antagonist | 1.34 | (0.92-1.94) | 1.52 | (0.78-2.98) |
| Non-TNF antagonist | 0.47 | (0.22-0.99) | 0.74 | (0.27-2.02) |
| Prophylaxis: Concomitant | 0.93 | (0.63-1.37) | 0.94 | (0.42-2.10) |
| immunomodulator | | | | |
| Surgical resection histopathology | | | | |
| Length of small bowel resected | 1.02 | (1.01-1.02) | 1.01 | (0.99-1.03) |
| Clean resection margins | 0.78 | (0.44-1.40) | 1.14 | (0.43-3.03) |
| Lymph node granuloma | 1.69 | (0.99-2.89) | 1.43 | (0.61-3.34) |
| Time to start post-operative | 1.00 | (0.99-1.02) | 1.03 | (0.98-1.01) |
| prophylaxis after surgery | | | | |

^{δ}Adjusted for age, sex, disease duration, pre-operative smoking status, and number of prior biologic exposures
^{†}Adjusted for age, sex, disease duration, pre-operative smoking status, and internal penetrating disease behavior
aHR, adjusted hazards ratio;
aOR, adjusted odds ratio;
CI, confidence interval

### Example 6: Granulomas, Greater Number of Prior Biologic Failures, and IL21 Genetic Variant are Associated with Decreased Persistence of Post-Operative Prophylaxis in Crohn's Disease

A retrospective, single center study of surgically naïve CD subjects >16 years undergoing ileocecal and/or small bowel resection between Jan. 1, 2000-12/31/2021 and prescribed post-operative prophylaxis with immunomodulator and/or biologic. Subjects ≥16 years undergoing their first ileocecal or small bowel resection (ICR and SBR, respectively) between Jan. 1, 2000-12/31/2021 and were prescribed an immunomodulator and/or biologic for post-operative pharmacologic prophylaxis.

The primary outcome was time to prophylaxis discontinuation (i.e. persistence of post-operative prophylaxis), a composite outcome defined as requiring recurrent surgery for POR or discontinuation of therapy due to persistent POR despite optimized drug concentration level or dose escalation, immunogenicity, and/or adverse event. Patients were followed from time of starting post-operative prophylaxis until occurrence of primary study outcome, lost to follow-up, or study completion (12/31/2021).

IBD serologies (ELISA) and genetic data (Immunochip) were generated. Cox proportional hazard analyses were adjusted for demographic characteristics and population structure. Post-operative prophylaxis was defined as an immunomodulator and/or biologic started after the primary anastomosis or ileostomy reversal with restoration of bowel continuity without objective evidence by imaging or endoscopic exam demonstrating recurrence. Timing of post-operative prophylaxis was defined by when a subject received the first dose based on provider documentation. Subjects who discontinued their prophylaxis for the following reasons were excluded: self-discontinuation/poor adherence, insurance denial, de-escalation of therapy, and unknown reasons. Through manual chart review, reasons for discontinuation were classified as persistent POR confirmed by ileocolonoscopy or imaging despite optimized drug concentration level or dose escalation (in those without available drug levels) as determined by the treating provider, immunogenicity (confirmed based on undetectable drug concentration level with detectable anti-drug antibodies in addition to stated reason for discontinuation by treating physician), and adverse events (i.e. infection, allergic reaction, infusion reaction, or malignancy). Patients who had POR and found to have low drug level on reactive therapeutic drug monitoring and received dose escalation were followed until their IBD provider switched the individual to another biologic agent because of persistent recurrence.

IBD-associated serologies (anti-Saccharomyces cerevisiae antibodies (IgA and IgA ASCA), anti-nuclear cytoplasmic antibody (ANCA), anti-flagellin (CBir1), anti-Pseudomonas fluorescence associated sequence I2 (I2) and anti-outer membrane porin C (OmpC)) from subject sera were measured by enzyme-linked immunosorbent assay. To evaluate identify clinical factors associated with drug persistence of post-operative prophylaxis, a survival analysis using multivariable Cox proportional hazard regression was performed adjusting for age, sex, CD disease duration, pre-operative smoking status, number of prior biologic failures, and internal penetrating disease behavior, which were defined a priori. Serologic data was generated via enzyme-linked immunosorbent assay (ELISA) and multivariable cox proportional hazard regression was adjusted for sex, and age at diagnosis.

Subjects with European ancestry were included in the analysis. Genotyping of IBD-susceptibly genes was performed at CSMC using Illumina Infinium ImmunoChip-v1 array. Genotype determinations (allele calls) were made using GenomeStudio version 2011.1 with Genotyping Module Version 1.9.4. single nucleotide polymorphism (SNP) s underwent methodological review and were evaluated using several SNP statistic parameters, including SNP call frequency, cluster separation, replicate and heritability error rates, heterozygous excess, theta mean and deviation, and R intensity mean. Raw genotyping intensities were visually examined to ensure accurate allele-calling for reported SNPs. The average genotyping rate across all samples that passed genotyping QC was 99.82%. Approximately 2% samples were genotyped in replicate (with both inter- and intra-plate replicates) and yielded >99.9% concordance for genotypes called. The association between drug persistence of post-operative prophylaxis and single SNPs was evaluated with Cox proportional hazard regression model using an R package called gwasurvivr 1.14.0. A nominal level of significance of p<0.05 for the known IBD loci and p<10e-5 for the other SNPs in ImmunoChip was specified. Genome-wide level of significance was defined as p<10e-8. Regression analysis was adjusted for sex, age at diagnosis, and first four principal components for population stratification. Subjects were also evaluated for HLA-DQA1*05 carrier status, which was inferred using HLA imputation on the Michigan server based on the dense genotyping of the HLA region on the Illumina ImmunoChip.

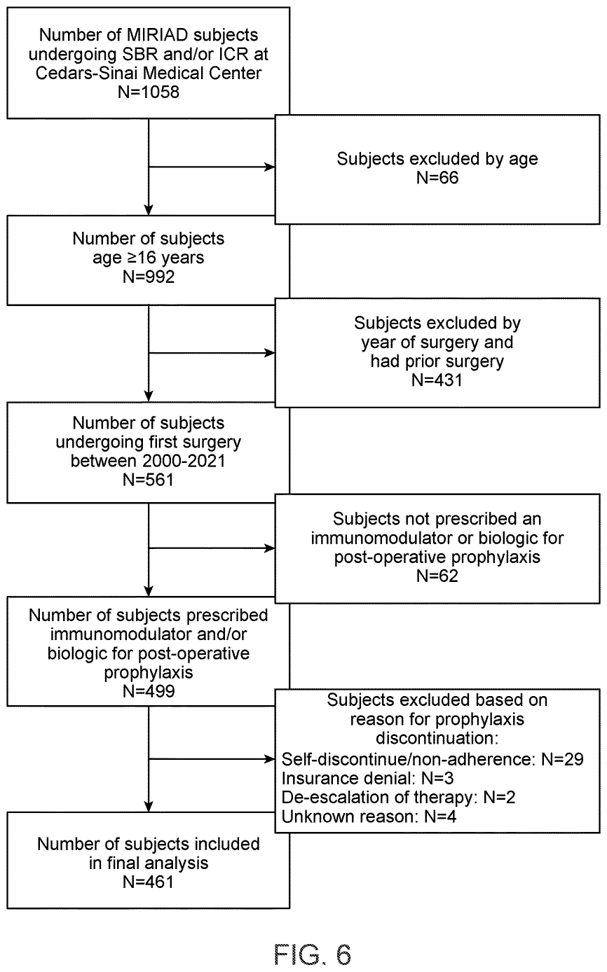

The study included 461 subjects, the inclusion and exclusion criteria of which is summarized in FIG. 6. The median follow-up time was 75.7 (IQR 33.1-132.2) months. Rates of prophylaxis discontinuation were 8.8% at 6 months, 16.4% at 12 months, and 35.0% at 36 months. The median time to discontinuation was 56.2 months. In the cohort, 73.4% (n=324) underwent an ileocolonoscopy within 15 months after surgery, and, of these subjects, 38.9% (n=126) developed Rutgeert's score >i2 recurrence. Pre-operatively, 47.8% (n=281) subjects were on biologics at the time of surgery and 27.5% (n=127) experienced at least one biologic failure prior to surgery. Again, prior biologics failure did not include biologic agents at the time of surgery due to the uncertainty between true non-response vs. disease that was too advanced to respond to medical therapy. Post-operatively, persistent POR despite optimized drug dose/level was the most common reason for prophylaxis discontinuation (58.4%, n=149) followed by adverse event (20.8%, n=53) and immunogenicity (11.0%, n=28). Of the entire cohort, 7.1% (n=18) required recurrent surgery for POR.

Active smoking (adjusted hazard ratio (aHR) 1.72, 95% (confidence interval) CI [1.12-2.66]), greater number of prior biologic failures (1 prior biologic failure (aHR 1.55, 95% CI [1.13-2.13]; >2 prior biologic failures (aHR 1.78, 95% CI [1.07-2.95]), and lymph node granulomas (aHR 1.59, 95% CI [1.07-2.35]) were associated with decreased persistence of post-operative prophylaxis. An IL21 gene variant met genome wide-level of significant association with decreased persistence of post-operative prophylaxis (aHR 1.89, p=4.70e-8). The study identified clinical, serologic, and genetic associations with decreased persistence of post-operative prophylaxis, notably a novel association with the IL21 gene.

The 461 subjects had serologic data available for analysis. Increased ANCA titer levels were associated with shorter time to prophylaxis discontinuation (aHR 1.01, p=0.02) while IgA ASCA titer levels were negatively associated with time to prophylaxis discontinuation (aHR 0.995, p=0.04). CBir1 (p=0.75), I2 (p=0.32),

Genetic analysis included 330 subjects. Following adjustment for population structure, multiple SNPs associated with the IL21 locus met genome-wide level of significance associated with decreased persistence of post-operative prophylaxis (lead SNP rs6811007, aHR 1.89, p=4.70e-8. FIG. 3 illustrates multiple SNPs associated with the IL21 are in linkage disequilibrium on chromosome 4. Because multiple SNPs tagging IL21 are in linkage disequilibrium and associated with decreased prophylaxis persistence, conditional analysis was performed to determine if only the lead SNP or multiple other SNPs associated with IL21 are independently associated with decreased persistence of post-operative prophylaxis. After conditional analysis, a second SNP tagging the IL21 locus is independently associated with decreased persistence of post-operative prophylaxis (rs1533236, p_{conditioned}=0.005 was identified. In additional to IL21, additional genetic associations that met nominal level of significance associated with persistence of post-operative prophylaxis (p<10-5) for genes involved in DNA mismatch repair (MSH3), encoding protein that promotes phagocytosis and has a role in apoptosis (ELMO1), and codes of LDL-receptor protein (LRP12).

In active luminal CD, presence of granulomas is associated with more aggressive disease behavior, need for biologic agents, and need for surgery. Post-operatively, presence of granulomatous disease has been associated with increased risk of POR, supporting an aggressive phenotype of CD. This study indicates the presence of granulomas reflects an underlying systemic process that contributes to aggressive and medically resistant CD.

TABLE 13
Cohort Demographics and Disease Characteristics.

| Variable | Total n = 461 | |
|---|---|---|
| Median age (years, interquartile range (IQR)) | 28.7 | (21.9-39.4) |
| Female, n(%) | 214 | (46.4) |
| European ancestry, n(%) | 419 | (91.1) |
| Median BMI at surgery (kg/m², IQR) | 22.1 | (19.0-25.9) |
| Median disease duration (months, IQR) | 67.0 | (16.4-124.0) |
| Median age at diagnosis (years, IQR) | 21.7 | (16.4-31.3) |
| Active smoking at surgery, n(%) | 33 | (7.4) |
| Crohn's disease location, n(%) | | |
| Ileal (L1) | 228 | (49.6) |
| Ileocolonic (L3) | 232 | (50.4) |
| Upper GI (L4) | 46 | (10.0) |
| Crohn's disease behavior, n(%) | | |
| Non-fibrostenosing, non-penetrating (B1) | 24 | (5.2) |
| Fibrostenosing (B2) | 227 | (49.5) |
| Internal penetrating (B3) | 95 | (20.7) |
| Fibrostenosing & penetrating (B2/3) | 113 | (24.6) |
| Perianal Crohn's disease, n(%) | 103 | (22.3) |
| # of biologic failures, n(%) | | |
| 0 | 334 | (72.5) |
| 1 | 94 | (20.4) |
| ≥2 | 2 | (7.2) |
| Pre-operative medication, n(%) | | |
| None | 69 | (15.1) |
| Steroid | 151 | (33.1) |
| 5ASA | 106 | (23.2) |
| Immunomodulator | 148 | (32.5) |
| Biologic | 218 | (47.8) |
| Post-operative Prophylaxis Therapy | | |
| Immunomodulator monotherapy | 170 | (36.9) |
| Biologic monotherapy | 235 | (51.0) |
| Biologic + immunomodulator combination | 56 | (12.1) |
| Surgical resection histopathology | | |
| Median length of small bowel resected (cm, IQR) | 22.8 | (14.6-34.1) |
| Histologically clean resection margins, n(%) | 304 | (86.6) |
| Lymph node granuloma, n(%) | 64 | (18.6) |
| Median time to start prophylaxis post-operatively | 4.3 | (1.4-6.3) |
| (weeks, IQR) | | |
| Reason for prophylaxis failure, n(%) | | |
| Indeterminate | 13 | (5.1) |
| Failure despite optimized dose/drug level | 149 | (58.4) |
| Immunogenicity | 28 | (11.0) |
| Adverse event | 53 | (20.8) |
| Recurrent surgery | 18 | (7.1) |

IQR, interquartile range

TABLE 14
Clinical factors associated with prophylaxis drug persistence

| Clinical Variable | aHR (95% CI) |
|---|---|
| Age | 1.003 (0.993-1.013) |
| Female | 1.066 (0.828-1.371) |
| European ancestry | 1.402 (0.865-2.272) |
| BMI | 1.017 (0.981-1.054) |
| Active smoking | 1.721 (1.115-2.655) |
| Disease duration at surgery | 1.000 (0.998-1.001) |
| Disease location | |
| Ileal (L1) | Reference |
| Ileocolonic (L3) | 1.413 (1.090-1.831) |
| Upper GI (L4) | 1.016 (0.649-1.591) |
| Disease Behavior | |
| Non-fibrostenosing, non-penetrating (B1) | Reference |
| Fibrostenosing (B2) | 0.905 (0.525-1.559) |
| Internal penetrating (B3) | 1.007 (0.566-1.791) |
| Fibrostenosis & internal penetrating (B2/3) | 0.761 (0.426-1.360) |
| Perianal disease | 0.935 (0.689-1.268) |
| Number of prior biologic failures | |
| 0 | Reference |
| 1 | 1.548 (1.128-2.126) |
| ≥2 | 1.779 (1.074-2.949) |
| Resection histopathology | |
| Resection length | 1.007 (1.002-1.011) |
| Clean resection margin (microscopically) | 0.864 (0.551-1.353) |
| Presence of lymph node granuloma | 1.587 (1.069-2.354) |
| Post-operative prophylaxis therapy | |
| Immunomodulator monotherapy | Reference |
| Biologic monotherapy | 0.953 (0.719-1.264) |
| Biologic + immunomodulator therapy | 0.892 (0.598-1.330) |

aHR, adjusted hazards ratio;
CI, confidence interval;
BMI, body mass index

TABLE 15
Genetic Associations of post-operative prophylaxis
with IL21 meeting genome-wide level of significance

| | | | Minor | | | |
|---|---|---|---|---|---|---|
| | | Position | allele | | Hazard | p- |
| SNP | Chromosome | (GRCh38.p14) | frequency | Gene | ratio | value |
| rs6811007 | 4 | 122664207 | 0.22 | IL21 | 1.89 | 4.66e−8 |
| rs6874652 | 5 | 80834893 | 0.13 | MSH3 | 1.91 | 1.01e−5 |
| rs1882083 | 7 | 36930569 | 0.19 | ELMO1 | 0.53 | 2.41E−5 |
| rs7823671 | 8 | 104588126 | 0.12 | LRP12 | 2.00 | 3.01e−5 |

IL21 = Interleukin 21, NCBI Gene: 59067
MSH3 = MutS Homolog 3, NCBI Gene: 4437
ELMO1 = Engulfment And Cell Motility 1, NCBI Gene: 9844
LRP12 = LDL Receptor Related Protein 12, NCBI Gene: 29967

TABLE 16
IBD serological associations with prophylaxis drug persistence

| | Titer level | Seropositivity |
|---|---|---|
| IBD Serology | (aHR, p-value) | (aHR, p-value) |
| ANCA | 1.01, 0.02 | 1.34, 0.20 |
| Cbir-1 | 1.00, 0.75 | 1.02, 0.91 |
| I2 | 1.00, 0.32 | 1.15, 0.47 |
| OmpC | 1.00, 0.67 | 1.19, 0.32 |
| IgA ASCA | 0.995, 0.04 | 0.78, 0.12 |
| IgG ASCA | 0.997, 0.33 | 0.82, 0.20 |
| ANCA | 1.01, 0.02 | 1.34, 0.20 |
| Cbir-1 | 1.00, 0.75 | 1.02, 0.91 |
| I2 | 1.00, 0.32 | 1.15, 0.47 |
| OmpC | 1.00, 0.67 | 1.19, 0.32 |
| IgA ASCA | 0.995, 0.04 | 0.78, 0.12 |
| IgG ASCA | 0.997, 0.33 | 0.82, 0.20 |
| ANCA | 1.01, 0.02 | 1.34, 0.20 |
| Cbir-1 | 1.00, 0.75 | 1.02, 0.91 |
| I2 | 1.00, 0.32 | 1.15, 0.47 |
| OmpC | 1.00, 0.67 | 1.19, 0.32 |
| IgA ASCA | 0.995, 0.04 | 0.78, 0.12 |
| IgG ASCA | 0.997, 0.33 | 0.82, 0.20 |
| ANCA | 1.01, 0.02 | 1.34, 0.20 |
| Cbir-1 | 1.00, 0.75 | 1.02, 0.91 |
| I2 | 1.00, 0.32 | 1.15, 0.47 |
| OmpC | 1.00, 0.67 | 1.19, 0.32 |
| IgA ASCA | 0.995, 0.04 | 0.78, 0.12 |
| IgG ASCA | 0.997, 0.33 | 0.82, 0.20 |

aHR, adjusted hazards ratio

TABLE 17
Conditional analysis of SNPs associated with IL21 locus reveals
a secondary SNP associated with prophylaxis drug persistence

| | Position | Minor | | | p-value after |
|---|---|---|---|---|---|
| | on Chromo- | allele | Hazards | | conditional |
| SNP | some 4 | frequency | ratio | p-value | analysis |
| rs13132308 | 123551114 | 0.10 | 1.43 | 0.04 | 0.11 |
| rs28517551 | 123561459 | 0.08 | 1.90 | 5.20e⁻⁴ | 0.77 |
| rs1533236 | 123564458 | 0.47 | 1.32 | 0.01 | 5.14E−03 |
| rs7678116 | 123571118 | 0.21 | 1.88 | 1.48e⁻⁷ | 0.44 |
| rs12511287 | 123578531 | 0.42 | 1.38 | 4.51e⁻³ | 0.56 |
| rs4833253 | 123578850 | 0.23 | 1.75 | 2.28e⁻⁶ | 0.40 |
| rs6844999 | 123581235 | 0.22 | 1.90 | 9.25e⁻⁸ | 1.00 |
| rs6825988 | 123582081 | 0.45 | 1.37 | 0.02 | 0.31 |
| rs4833841 | 123584597 | 0.21 | 1.87 | 1.94e⁻⁶ | 0.90 |
| rs6811007 | 123585362 | 0.22 | 1.90 | 9.25e⁻⁸ | 1.00 |
| rs9685849 | 123588318 | 0.23 | 1.86 | 8.68e⁻⁷ | 0.20 |
| rs925550 | 123588526 | 0.23 | 1.72 | 4.12e⁻⁶ | 0.17 |
| rs6837455 | 123588942 | 0.22 | 1.89 | 1.41e⁻⁷ | 0.99 |
| rs6534357 | 123589353 | 0.23 | 1.71 | 5.38e⁻⁶ | 0.11 |
| rs1398559 | 123593320 | 0.34 | 1.46 | 1.76e⁻³ | 0.87 |
| rs12501008 | 123597440 | 0.23 | 2.12 | 8.83e⁻⁶ | 0.51 |
| rs7664318 | 123599124 | 0.23 | 1.69 | 6.14e⁻⁶ | 0.12 |
| rs75802552 | 123599326 | 0.36 | 1.41 | 0.01 | 0.12 |
| rs60318098 | 123599898 | 0.22 | 1.86 | 1.86e⁻⁷ | 0.74 |

### Example 7: Pre-Operative Visceral Adipose Tissue (VAT) Quality but not Quantity was Associated with Post-Operative Recurrence (POR) in Crohn's Disease

A further analysis of the retrospective study conducted in Example 2 was performed, which refined the relevant subjects considered in the study, and which provides further data on the subjects analyzed.

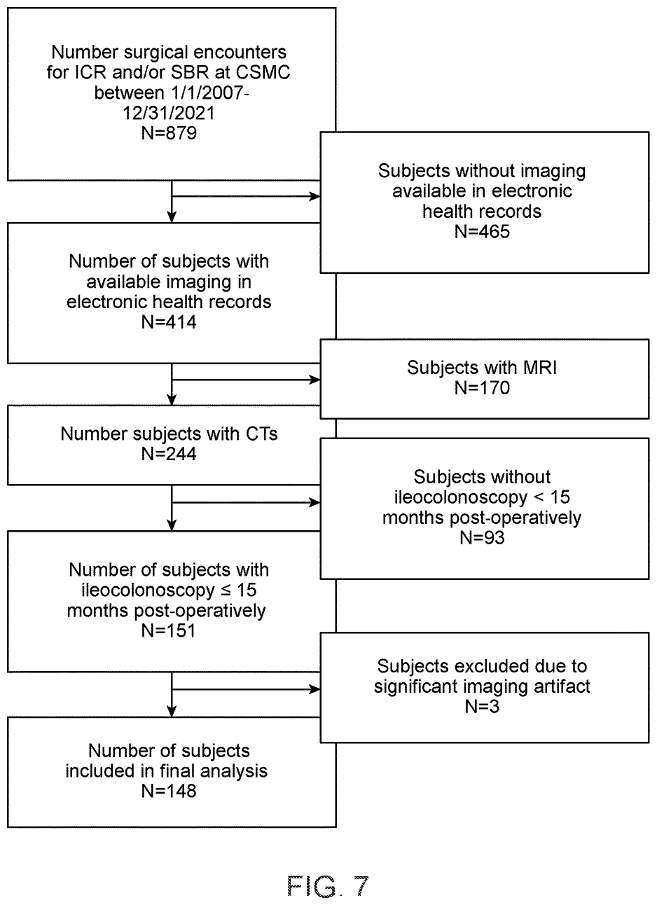

Methods: A single center, retrospective study of adult and pediatric ileal and ileocolonic CD subjects undergoing ileocecal or small bowel resection between Jan. 1, 2007 to Dec. 31, 2021 was conduced. Subjects with pre-operative CT abdomen/pelvis <12 months of surgery and an ileocolonoscopy≤15 months after surgery were included. FIG. 7 summarizes inclusion and inclusion criteria. To align with clinical practice, CTs performed within 12 months of surgery were selected because imaging within 12 months is clinically sufficient to make surgical decisions. 3D measurements of VAT volume (cm) and ratio of VAT to subcutaneous adipose tissue (SAT) volume (VAT:SAT) were obtained from CT. VAT radiodensity (HU) and ratio of VAT:SAT radiodensity were obtained from CT. CTs performed within 12 months of surgery were selected because imaging within 12 months is clinically sufficient to make surgical decisions. Furthermore, MRIs were excluded because CT scans offer additional radiographic parameters, such as radiodensity, that is not possible with MRI.

Baseline demographics, disease-related characteristics, and CD medications at the time of surgery were recorded via manual chart review. Disease location and disease behavior were classified by the Montreal classification system. Post-operatively prophylaxis status was also recorded and was defined as CD-directed therapy started after the primary anastomosis or ileostomy reversal with restoration of bowel continuity but before the first post-operative ileocolonoscopy. Rutgeert's scores were recorded from ileocolonoscopy procedure reports. When a Rutgeert's score was not recorded, text and images of endoscopy reports were reviewed by a board-certified gastroenterologist with advanced IBD training (PG) to retrospectively assign a Rugeert's score. Rutgeert's score i2 was not differentiated into i2a and i2b because this was not standard of practice during the early part of the study period and could not be reliably distinguished retrospectively based on text or images.

### Acquisition of Radiographic Mesenteric Parameters

The primary exposures were the radiographic mesenteric parameters of interest, which included visceral adipose tissue (VAT) volume (cm³), ratio of VAT: subcutaneous adipose tissue (SAT) volume, VAT radiodensity (Hounsfield units [HU]), ratio of VAT:SAT ratio, severity of vasa recta engorgement (VR), and mesenteric lymphadenopathy.

VAT parameters: To generate VAT parameters, all CT scans were processed with Vitrea® Advanced Visualization Platform (Canon Medical Systems), which performs semiautomated segmentation of the abdominal VAT and SAT compartments using attenuation thresholds between −50 and −150 HU. A representative CT scan image is depicted in FIG. 4. Of note, the radiodensity of adipose tissue is −150 to −50 HU, and lower adipose radiodensity is suggested to reflect poorer fat quality.

VAT and SAT parameters were measured from the first lumbar vertebra (L1) to the fifth (L5), as previously described. The boundaries of automated CT outlined adipose tissue compartments were manually reviewed and corrected for any errors in each slice. After segmentation, VAT and SAT volumes and radiodensity were automatically calculated in Vitrea®, which were then used to calculated VAT:SAT volume and radiodensity. All VAT and SAT segmentation were performed by a certified advanced imaging analyst (NG) who was blinded to the clinical data and outcomes. Three subjects had significant image artifact that precluded VAT and SAT analysis and was excluded from the final analysis.

VAT parameters: To generate VAT parameters, all CT scans were processed with Vitrea® Advanced Visualization Platform (Canon Medical Systems), which performs semiautomated segmentation of the abdominal VAT and SAT compartments using attenuation thresholds between −50 and −150 HU. Supplemental FIG. 1 illustrates an example of the segmentation. VAT and SAT parameters were measured from the first lumbar vertebra (L1) to the fifth (L5), as previously described.24 The boundaries of automated CT outlined adipose tissue compartments were manually reviewed and corrected for any errors in each slice. After segmentation, VAT and SAT volumes and radiodensity were automatically calculated in Vitrea®, which were then used to calculated VAT:SAT volume and radiodensity. All VAT and SAT segmentation were performed by a certified advanced imaging analyst (NG) who was blinded to the clinical data and outcomes. Three subjects had significant image artifact that precluded VAT and SAT analysis and was excluded from the final analysis.

Severe vasa recta engorgement: Severity of vasa recta engorgement was determined by calculating the ratio of the diameter of the vasa recta supplying the inflamed bowel intended for resection to the diameter of the vasa rectal supplying health bowel. Severe vasa recta engorgement was defined as diameter of vasa recta of the inflamed bowel >2× the diameter of the vasa recta of the healthy bowel as previously described. 17 Vasa recta diameters were manually measured in the transverse plane of the CT by PG.

Mesenteric lymphadenopathy: Each CT scan was reviewed by PG to locate and measure the largest mesenteric lymph node in the region of the inflamed bowel intended for resection. Mesenteric lymphadenopathy was defined as the largest lymph node measuring >10 mm along the short axis in the transverse plan of the CT as previously described. Radiographic vasa recta and lymph node measurements from 48 images (32% of cohort) were reviewed and confirmed with a board-certified body imaging radiologist with over 20 years of experience (CK) to ensure accuracy and consistency.

The primary outcome was early endoscopic POR (ePOR) defined as Rutgeert's score>i2 on colonoscopy <15 months after surgery. The secondary outcome was ePOR severity (Rutgeert's score i0-4). To assess the association between the VAT metrics and early ePOR and ePOR severity, binomial and ordinal logistic regressions were performed, respectively, adjusting for demographic and disease related variables to calculate adjusted odds ratio (aOR) and 95% confidence interval(CI). Adjustment for established POR risk factors were determined a priori and included age at surgery, sex, time from CD diagnosis to surgery, internal penetrating disease behavior, post-operative prophylaxis status, and prior CD-related surgery.

Results: A total of 148 subjects were included in the final analysis. Of the entire cohort, 45.3% (n=67) developed early ePOR, and the median time between surgery and first post-operative ileocolonoscopy was 32.86 (IQR 26.00-43.29) weeks. The median amount of time between CT and surgery was 1.10 (interquartile range 0.34-2.80) months. On binomial logistic regression, VAT radiodensity (adjusted odds ratio (aOR): 0.62, 95% confidence interval (CI) [0.41-0.92]) and VAT:SAT radiodensity (aOR 7.38, 95% CI [1.43-38.15]) were associated with early ePOR. VAT volume (aOR: 1.22, 95% CI [0.78-1.92]), while VAT:SAT volume (aOR: 0.71, 95% CI [0.46-1.08]) was not associated with early ePOR. Of the subjects who did not develop ePOR, 33.8% (n=50) and 20.9% (n=31) had Rugeert's i0 and i1, respectively. Of the subjects who developed ePOR, majority of patients who developed early ePOR had Rugeert's i2 (29.7%, n=44) while 9.5% (n=14) and 6.1% (n=9) had Rutgeert's i3 and i4, respectively. On ordinal logistic regression, VAT radiodensity (aOR 0.64, 95% CI [0.46-0.90]) and VAT:SAT radiodensity (aOR: 5.15, 95% CI [1.31-20.22]) were associated with severity ePOR. VAT volume (aOR: 1.10, 95% CI [0.77-1.59]), VAT:SAT volume (aOR: 0.83, 95% CI [0.59-1.18]) was not associated with ePOR severity.

Lower radiodensity of VAT and increased VAT:SAT radiodensity were independently associated with increased risk of early ePOR and more severe ePOR.

### Statistical Analysis

Descriptive statistics were used to examine baseline cohort demographics and disease-related characteristics. For variables not normally distributed based on visual assessment, a rank-based inverse normalized transformation was applied to the variable. Univariate regression was used to assess demographic and disease-related associations with radiographic mesenteric parameters. To determine independent associations between the radiographic mesenteric parameters and early ePOR and severity of ePOR, multivariable binomial and ordinal logistic regressions were performed, respectively. Adjustment for established POR risk factors were determined a priori and included age at surgery, sex, time from CD diagnosis to surgery, internal penetrating disease behavior, post-operative prophylaxis status, and prior CD-related surgery.6 Active smoking was not included in the models because only 4.2% subjects (n=6) were active smokers in the cohort. P-value <0.05 was considered statistically significant. All statistical analyses were performed using SPSS (IBM Corp. Released 2016. IBM SPSS Statistics for Windows, Version 24.0. Armonk, NY: IBM Corp.).

### Patient Demographics

A total of 148 subjects were included in the final analysis (FIG. 7). The median amount of time between CT and surgery was 1.10 (interquartile range 0.34-2.80) months. The below Table 18 summarizes cohort demographics. Only 4.2% (n=6) were active smokers at the time of surgery. Pre-operatively, majority of patients had fibrostenosing (B2) disease behavior (38.8%, n=57), and 52% (n=77) were on a biologic agent before surgery. Additionally, 29.7% (n=44) had at least one prior CD-related surgery. Post-operatively, 87.2% (n=129) of subjects started post-operative prophylaxis with biologic agents being the most common (76.7%, n=99).

On univariate analysis, there were several notable associations with the radiographic mesenteric parameters of interest (Table 19). VAT volume was associated with older age (β: 0.49, p=2.72e-10), longer time to surgery from CD diagnosis (β: 0.24, p=0.003) and BMI (β: 0.70, p=2.06e-20). VAT:SAT volume was associated with older age (β: 0.25, p=0.003) and negatively associated with female sex (β: −0.29, p=4.83e-4). VAT radiodensity was not associated with age (p=0.62) or sex (p=0.62) but negatively associated with BMI (β: −0.18, p=0.04) and prior surgery (β: −0.17, p=0.04). Finally, severe vasa recta engorgement was associated with older age (odds ratio (OR): 0.98, p=0.04), perianal disease (OR: 5.72, p=0.02), and prior surgery (OR: 0.37, p=0.01). Active smoking, disease location, and behavior were not associated with any of the radiographic mesenteric parameters. Similarly, VAT:SAT radiodensity and mesenteric lymphadenopathy were not associated with any demographics or disease-related factors.

TABLE 18
Cohort demographics (n = 148)

| | Total n = 148 | |
|---|---|---|
| Mean Age (years, SD) | 38.9 | (17.2) |
| Female, n(%) | 69 | (46.6) |
| European Ancestry, n(%) | 129 | (88.4) |
| Mean disease duration, (months, SD) | 152.1 | (133.7) |
| Mean body mass index (kg/m², SD) | 23.1 | (4.8) |
| Active smoking, n(%) | 6 | (4.2) |
| Disease location, n(%) | | |
| Ileal (L1) | 66 | (44.6) |
| Ileocolonic (L3) | 83 | (55.4) |
| Upper GI (L4) | 14 | (9.5) |
| Disease behavior, n (%) | | |
| Non-fibrostenosing, non-penetrating (B1) | 8 | (5.4) |
| Fibrostenosing (B2) | 57 | (38.8) |
| Internal penetrating (B3) | 37 | (25.2) |
| Stricturing and internal penetrating (B3) | 45 | (30.6) |
| Perianal disease, n(%) | 26 | (17.6) |
| Pre-operative medication, n(%) | | |
| None | 35 | (23.6) |
| Steroid | 51 | (34.5) |
| 5-aminosalicylic acid | 22 | (14.9) |
| Immunomodulator | 35 | (23.6) |
| Biologic | 77 | (52.0) |
| Prior surgery, n(%) | 44 | (29.7) |
| Post-operative prophylaxis, n(%) | 129 | (87.2) |
| 5-aminosalicylic acid | 8 | (6.2) |
| Immunomodulator | 23 | (17.8) |
| Biologic | 99 | (76.7) |

TABLE 19
Univariate association of demographic and disease-related
factors with radiographic mesenteric parameters.

| | | | | | Severe vasa | |
|---|---|---|---|---|---|---|
| | VAT | VAT:SAT | VAT | VAT:SAT | recta | Mesenteric |
| | volume | volume | radiodensity | radiodensity | engorgement | lymphadenopathy |
| | (β, p-value) | (β, p-value) | (β, p-value) | (β, p-value) | (OR, p-value) | (OR, p-value) |
| Age | 0.49, | 0.25, 0.003 | −0.04, 0.62 | 0.04, 0.64 | 0.98, 0.04 | 1.01, 0.53 |
| | 2.72e⁻¹⁰ | | | | | |
| Female | −0.15, 0.08 | −0.29, 4.83e⁻⁴ | 0.07, 0.39 | −0.1, 0.25 | 0.72, 0.38 | 1.00, 1.00 |
| European | 0.05, 0.59 | 0.09, 0.28 | 0.04, 0.62 | −0.06, 0.46 | 1.65, 0.36 | 0.58, 0.34 |
| Ancestry | | | | | | |
| Disease | 0.24, 0.003 | 0.11, 0.19 | −0.08, 0.35 | 0.02, 0.86 | 1.0, 0.56 | 1.00, 0.92 |
| duration | | | | | | |
| BMI | 0.70, | 0.11, 0.22 | −0.18, 0.04 | 0.15, 0.09 | 0.96, 0.25 | 0.99, 0.77 |
| | 2.06e⁻²⁰ | | | | | |
| Active | 0.12, 0.148 | −0.02, 0.81 | 0.02, 0.82 | 0.02, 0.79 | 1.78, 0.60 | 4.27, 0.09 |
| smoking | | | | | | |
| CD | | | | | | |
| location | | | | | | |
| L3vs. | 0.02, 0.78 | 0.02, 0.83 | 0.03, 0.77 | −0.04, 0.65 | 1.40, 0.36 | 2.11, 0.09 |
| L1 | | | | | | |
| L4 | −0.20, 0.018 | −0.06, 0.51 | −0.05, 0.52 | 0.09, 0.37 | 1.40, 0.62 | 1.08, 0.91 |
| CD | | | | | | |
| behavior | | | | | | |
| B1 | Reference | Reference | Reference | Reference | Reference | Reference |
| B2 | 0.08, 0.53 | 0.16, 0.20 | −0.18, 0.15 | 0.15, 0.23 | 0.18, 0.12 | 1.71, 0.63 |
| B3 | −0.04, 0.80 | 0.08, 0.61 | −0.15, 0.34 | 0.17, 0.27 | 0.74, 0.79 | 2.96, 0.34 |
| B2/3 | 0.11, 0.43 | 0.23, 0.10 | −0.27, 0.06 | 0.26, 0.07 | 0.68, 0.73 | 1.26, 0.84 |
| Perianal | −0.16, 0.06 | −0.04, 0.68 | 0.05, 0.56 | −0.02, 0.81 | 5.72, 0.02 | 1.48, 0.42 |
| disease | | | | | | |
| Prior | 0.09, 0.29 | 0.07, 0.38 | −0.17, 0.04 | 0.10, 0.24 | 0.37, 0.01 | 0.55, 0.23 |
| surgery | | | | | | |
| Pre-op | −0.04, 0.63 | −0.16, 0.06 | 0.01, 0.87 | 0.04, 0.64 | 1.56, 0.28 | 0.76, 0.57 |
| steroids | | | | | | |

VAT, visceral adipose tissue;
SAT, subcutaneous adipose tissue;
OR, odds ratio

TABLE 20
Multivariate regression of radiographic mesenteric parameters associated
with early endoscopic POR and severity of endoscopic POR

| | Early endoscopic POR | Severity of endoscopic POR |
|---|---|---|
| | aOR (95% CI) | aOR (95% CI) |
| VAT Volume | 1.22 (0.78-1.92) | 1.10 (0.77-1.59) |
| (cm³) | | |
| VAT:SAT | 0.71 (0.46-1.08) | 0.83 (0.59-1.18) |
| Volume | | |
| VAT | 0.62 (0.41-0.92) | 0.64 (0.46-0.90) |
| Radiodensity | | |
| (HU) | | |
| VAT:SAT | 7.38 (1.43-38.15) | 5.15 (1.31-20.22) |
| Radiodensity | | |
| Severe vasa | 1.42 (0.60-3.33) | 1.79 (0.84-3.80) |
| recta | | |
| engorgement | | |
| Mesenteric | 1.58 (0.66-3.75) | 2.01 (0.95-4.25) |
| lymphadenopathy | | |

Adjusted age at surgery, sex, time from CD diagnosis to surgery, internal penetrating disease behavior, post-operative prophylaxis status, and prior CD-related surgery

Conclusion: VAT radiodensity metrics are associated with early ePOR and ePOR severity in this cohort. These findings suggest VAT quality may be a novel prognostic marker for POR and provide insight into the underlying biology of POR.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed:

1. A method of treating Crohn's disease (CD) in a subject, the method comprising: administering to the subject a therapeutically effective amount of a therapeutic agent for CD, wherein one or more post-operative prophylaxis persistence (“POPP”) markers were detected in the subject.

2. The method of claim 1, further comprising determining the therapeutic agent to have prophylactic persistence in treating the CD in the subject based, at least in part, on the one or more POPP markers detected in the subject.

3. A method of determining prophylactic persistence of therapeutic agent in treating Crohn's disease (CD) in a subject that has undergone operation, the method comprising:
a) detecting one or more post-operative prophylaxis persistence (“POPP”) markers in the subject; and
b) determining a likelihood that the therapeutic agent exhibits the prophylactic persistence in treating the CD in the subject based, at least in part, on the one or more POPP markers detected in the subject, wherein the likelihood is compared with a control subject with the CD and that has received a surgical resection to treat the CD.

4. The method of any one of claims 1-3, further comprising detecting the one or more POPP markers from a sample obtained from the subject.

5. The method of claim 4, wherein the sample comprises blood, serum, plasma, sweat, hair, tears, urine, saliva, stool, or combination thereof.

6. The method of any one of claims 1-5, wherein the one or more POPP markers comprises one or more POPP clinical factors, one or more POPP serological factors, one or more POPP genetic factors, or any combination thereof.

7. The method of claim 6, wherein the one or more POPP clinical factors comprises an active smoker status, disease location, a failure of one or more therapeutic agents to treat the CD, resection length of operation, or lymph node granuloma, or any combination thereof.

8. The method of claim 7, further comprising obtaining the one or more POPP clinical factors from the subject directly or reviewing medical records of the subject to obtain the one or more POPP clinical factors.

9. The method of any one of claims 6-8, wherein the disease location is an ileocolonic region of an intestine of the subject.

10. The method of claim 9, wherein the likelihood of the POPP of the therapeutic agent is high based, at least in part, on the one or more POPP clinical factors detected in the subject comprising the disease location in the ileocolonic region of the intestine of the subject.

11. The method of any one of claims 7-10, wherein the likelihood of the POPP of the therapeutic agent is high based, at least in part, on the one or more POPP clinical factors detected in the subject comprising the active smoker status.

12. The method of claim 11, wherein an active smoker status is defined by smoking at least one cigarette per week.

13. The method of any one of claims 7-12, wherein the therapeutic agent has a low likelihood of the POPP based, at least in part, on the one or more POPP clinical factors detected in the subject comprising the lymph node granuloma.

14. The method of any one of claims 7-12, wherein the failure of the one or more therapeutic agents to treat the CD is a recurrence or flare up of the CD following successful remission of the CD following administration of the one or more therapeutic agents to the subject.

15. The method of claim 14, wherein the one or more therapeutic agents comprises a steroid, an immunomodulator, 5-aminosalicylate, or combinations thereof.

16. The method of claim 14, wherein the one or more therapeutic agents comprises one or more biologic therapeutic agents.

17. The method of claim 16, wherein the one or more biologic therapeutic agents comprises two or more biologic therapeutic agents.

18. The method of claim 17, wherein the one or more biologic therapeutic agents comprises three or more biologic therapeutic agents.

19. The method of any one of claims 6-18, wherein the one or more POPP serological factors comprises ANCA seronegativity, ASCA IgA seronegativity, or ASCA IgG seronegativity, or any combination thereof.

20. The method of claim 19, wherein the likelihood of the POPP of the therapeutic agent is high based, at least in part, on the one or more POPP serological factors detected in the subject comprising the ASCA seropositivity.

21. The method of claim 19, wherein the likelihood of the POPP of the therapeutic agent is high based, at least in part, on the one or more POPP serological factors detected in the subject comprising the ANCA seronegativity.

22. The method of claim 19, wherein the likelihood of the POPP of the therapeutic agent is high based, at least in part, on the one or more POPP serological factors detected in the subject comprising the ASCA seropositivity and the ANCA seronegativity.

23. The method of any one of claims 6-22, wherein the one or more POPP serological factors is detected in the subject by a method comprising introducing a sample obtained from the subject to a detection agent configured to bind ANCA, ASCA IgA, ASCA IgG under conditions sufficient to detect the ANCA, ASCA IgA, ASCA IgG, or any combinations thereof.

24. The method of any one of claims 6-22, further comprising detecting the one or more POPP serological factors comprising performing an enzyme-linked immunosorbent assay (ELISA).

25. The method of claim 24, further comprising measuring seropositivity or seronegativity, wherein the measuring the seropositivity or the seronegativity comprises calculating a percentage of a reference value from pooled sera obtained from a patient population diagnosed with CD.

26. The method of any one of claims 6-25, wherein the one or more POPP genetic factors comprises an absence of absence of an adenosine (“A”) at a polymorphism position comprising rs6811007, or a proxy polymorphism position in linkage disequilibrium therewith as determined with an r² of at least 0.85, or a combination thereof; or wherein the one or more POPP genetic factors comprises an absence of an adenosine (“A”) at a polymorphism position comprising rs1533236, or a proxy polymorphism position in linkage disequilibrium therewith as determined with an r² of at least 0.85, or a combination thereof; or wherein the one or more POPP genetic factors comprise both.

27. The method of claim 26, wherein the one or more POPP genetic factors is detected in the subject by a method comprising detecting a nucleic acid sequence.

28. The method of claim 27 wherein the detecting the nucleic acid sequence comprises performing quantitative polymerase chain reaction (qPCR), nucleic acid sequencing, or hybridization to immobilized nucleic acid sequences on a nucleic acid array.

29. The method of any one of claims 6-28, wherein the one or more POPP histological factors comprises operative visceral adipose tissue (VAT) quality, wherein the operative VAT is defined by a reduced radiodensity.

30. The method of claim 29, wherein the reduced radiodensity comprises a VAT radiodensity of less than or equal to about 0.631 Hounsfield unit (HU).

31. The method of claim 29, wherein the reduced radiodensity comprises a ratio of VAT to SAT radiodensity of up to 5.559.

32. The method of claim 29, wherein the reduced radiodensity comprises a VAT radiodensity of about 0.43 to about 0.7 HU.

33. The method of claim 29, wherein the reduced radiodensity comprises a ratio of VAT to SAT radiodensity of ranging from 1.18-6.

34. The method of any one of claims 29-33, wherein the likelihood of the POPP of the therapeutic agent is high based, at least in part, on the one or more POPP histological factors detected in the subject comprising the operative VAT quality.

35. A method of treating Crohn's disease (CD) in a subject, the method comprising: administering to the subject a therapeutically effective amount of a therapeutic agent for treatment of the CD, wherein one or more post-operative recurrence (“POR”) markers were detected in the subject.

36. The method of claim 35, further comprising determining the subject to have a likelihood of the POR based, at least in part, on the one or more POR markers detected in the subject relative to a control subject having the CD that has received a resection to treat the CD.

37. A method of determining the prognosis of post-operative recurrence (“POR”) of Crohn's disease (CD) in a subject, the method comprising:
a) detecting one or more POR markers in the subject; and
b) determining the subject to have a likelihood of POR based, at least in part on the one or more POR markers detected in the subject, relative to a control subject having the CD that has received a resection to treat the CD.

38. The method of claim 36, further comprising detecting the one or more POR markers from a sample obtained from the subject.

39. The method of claim 38, wherein the sample comprises blood, serum, plasma, sweat, hair, tears, urine, saliva, stool, or any combination thereof.

40. The method of any one of claims 35-39, wherein the one or more POR markers comprises one or more POR clinical factors, one or more POR serological factors, one or more POR genetic factors, or one or more POR histological factors, or any combination thereof.

41. The method of claim 40, wherein the one or more POR clinical factors comprises active smoking, disease location, a failure of a therapeutic agent for treatment of the CD, resection length of operation, or lymph node granuloma, or any combination thereof.

42. The method of claim 41, wherein the disease location is an ileocolonic region of an intestine of the subject.

43. The method of any one of claims 40-42, further comprising identifying the likelihood of the subject of the POR is high relative to the control subject, wherein the one or more POR clinical factors detected in the subject comprises the disease location, wherein the disease location is an ileocolonic region of an intestine of the subject.

44. The method of any one of claims 40-43, further comprising identifying the likelihood of the subject of the POR is low relative to the control subject, wherein the one or more POR clinical factors detected in the subject comprises disease location in the ileal region.

45. The method of any one of claims 41-44, wherein the likelihood is high relative to the control subject based, at least in part, on the one or more POR clinical factors detected in the subject, wherein the one or more POR clinical factors comprises an active smoker.

46. The method of claim 45, wherein an active smoker status is defined by smoking at least one cigarette per week.

47. The method of any one of claims 41-46, further comprising identifying the likelihood of the subject as of the POR is high relative to the control subject, wherein the one or more POR clinical factors detected in the subject comprises the lymph node granuloma.

48. The method of any one of claims 41-47, wherein the failure of the one or more therapeutic agents to treat the CD is a recurrence or flare up of the CD following successful remission of the CD following administration of the one or more therapeutic agents to the subject.

49. The method of any one of claims 41-48, wherein the one or more therapeutic agents comprises a steroid, an immunomodulator, 5-aminosalicylate, or combinations thereof.

50. The method of any one of claims 41-49, wherein the one or more therapeutic agents comprises one or more biologic therapeutic agents.

51. The method of any one of claims 41-50, wherein the one or more biologic therapeutic agents comprises two or more biologic therapeutic agents.

52. The method of any one of claims 41-50, wherein the one or more biologic therapeutic agents comprises three or more biologic therapeutic agents.

53. The method of any one of claims 41-52, further comprising identifying the likelihood of the subject of the POR is high relative to the control subject, wherein the one or more POR clinical factors detected in the subject comprises a failure of the one or more therapeutic agents to treat the CD, wherein the one or more therapeutic agents comprises one or more biologic therapeutic agents to treat the CD.

54. The method of any one of claims 40-53, wherein the one or more POR serological factors is detected in the subject by a method comprising introducing a sample obtained from the subject to a detection agent configured to bind ANCA, ASCA IgA, ASCA IgG under conditions sufficient to detect the ANCA, ASCA IgA, ASCA IgG, or any combinations thereof.

55. The method of any one of claims 40-54, further comprising identifying the subject as having a high likelihood of the POR wherein if the one or more POR clinical factors detected in the subject comprises ANCA seropositivity, ASCA IgA seropositivity, or ASCA IgG seropositivity, or any combination thereof.

56. The method of any one of claims 40-54, further comprising identifying the subject as having a high likelihood of the POR wherein if the one or more POR clinical factors detected in the subject comprises ASCA seropositivity.

57. The method of any one of claims 40-54, further comprising identifying the subject as having a high likelihood of the POR wherein if the one or more POR clinical factors detected in the subject comprises ANCA seronegativity.

58. The method of any one of claims 40-54, further comprising identifying the subject as having a high likelihood of the POR wherein if the one or more POR clinical factors detected in the subject comprises ASCA seropositivity and ANCA seronegativity.

59. The method of any one of claims 40-58, wherein the one or more POR genetic factors comprises an absence of an adenosine (“A”) at a polymorphism position comprising rs6811007, or a proxy polymorphism position in linkage disequilibrium therewith as determined with an r² of at least 0.85, or a combination thereof; or wherein the one or more POR genetic factors comprises an absence of an adenosine (“A”) at a polymorphism position comprising rs1533236, or a proxy polymorphism position in linkage disequilibrium therewith as determined with an r² of at least 0.85, or a combination thereof; or wherein the one or more POR genetic factors comprises both.

60. The method of claim 59, wherein the one or more POR genetic factors is detected in the subject by a method comprising detecting a nucleic acid sequence.

61. The method of claim 60, wherein the detecting the nucleic acid sequence comprises quantitative polymerase chain reaction (qPCR), nucleic acid sequencing, or hybridization to a nucleic acid array

62. The method of any one of claims 40-61, wherein the one or more POR histological factors comprises operative visceral adipose tissue (VAT) quality, wherein the operative VAT is defined by a reduced radiodensity.

63. The method of claim 62, wherein the increased radiodensity comprises a VAT radiodensity of at least 0.631 HU.

64. The method of claim 62, wherein the increased radiodensity comprises a VAT radiodensity of ranging from 0.631-0.926 HU.

65. The method of claim 62, wherein the increased radiodensity comprises a VAT:SAT radiodensity of at least 5.559.

66. The method of claim 62, wherein the increased radiodensity comprises a VAT:SAT radiodensity of ranging from 5.56-26.

67. The method of claim 40-66, wherein the one or more POR histological factors comprises operative visceral adipose tissue (VAT) quality.

68. The method of claim 67, wherein the likelihood of the POR is high relative to the control subject based, at least in part, on the one or more POR histological factors detected in the subject, wherein the one or more POR histological factors comprises the operative VAT quality, wherein operative VAT quality is defined by an increased radiodensity.

69. The method of any one of claims 1-68, wherein the therapeutic agent comprises an inhibitor of tumor necrosis factor alpha (TNFα), an inhibitor of Interleukin-23 (IL23), an inhibitor of integrin, an inhibitor of SIPR (Sphingosine-1-phosphate receptor), an inhibitor of TNF superfamily member 15 (TL1A), or an inhibitor of Janus kinase 2 (JAK), or any combination thereof.

70. The method of claim 69, wherein the inhibitor of tumor necrosis factor alpha (TNFα) comprises adalimumab, infliximab, golimumab, certolizumab, or etanercept, or any combination thereof.

71. The method of claim 69, wherein the inhibitor of IL23 comprises ustekinumab, guselkumab, risankizumab, brazikumab, mirikizumab, tildrakizumab, or briakinumab, or any combination thereof.

72. The method of claim 69, wherein the inhibitor of integrin comprises etrolizumab, vedolizumab, natalizumab, or ontamalimab, or any combination.

73. The method of claim 69, wherein the inhibitor of SIPR comprises fingolimod, siponimod, etrasimod, ozanimod, ponesimod, amiselimod, ceralifimod, or mocravimod, or any combination thereof.

74. The method of claim 69, wherein the inhibitor of TLIA comprises tulisokibart, RVT-3101/PF-06480605, or duvakitug, or any combination thereof.

75. The method of claim 69, wherein the inhibitor of JAK comprises upadacitinib, filgotinib, tofacitinib, abrocitinib, baricitinib, or any combination thereof.

76. The method of any one of claims 1-75, further comprising administering to the subject another therapeutic agent comprising an antagonist of interleukin 21 (IL-21).

77. The method of claim 76, wherein the antagonist of IL-21 comprises an antibody, or an antigen-binding fragment thereof.

78. A method of enriching a target nucleic acid in a sample, the method comprising:
a) providing a plurality of target nucleic acid sequences obtained from a biological sample from a subject with Crohn's disease (CD);
b) bringing a fluid reaction formulation comprising a plurality of synthetic oligonucleotide molecules in contact with the plurality of target nucleic acid sequences, wherein each synthetic oligonucleotide molecule of the plurality of synthetic oligonucleotide molecules is complementary to at least one target nucleic acid sequence of the plurality of target nucleic acid sequences;
c) hybridizing the plurality of synthetic oligonucleotide molecules to the plurality of synthetic oligonucleotide molecules;
d) amplifying the plurality of target nucleic acid sequences hybridized to the plurality of synthetic oligonucleotide molecules in (c), thereby enriching the plurality of target nucleic acid sequences in the fluid reaction formulation; and
e) detecting the plurality of target nucleic acid sequences that were enriched in (d), wherein the detecting in (e) of the plurality of target nucleic acid sequences is indicative of a high likelihood of post-operative prophylaxis persistence (“POPP”), as compared with a control subject.

79. The method of claim 78, further comprising detecting an absence of a polymorphism comprising an adenosine (“A”) at rs6811007, or a proxy polymorphism position in linkage disequilibrium therewith as determined with an r² of at least 0.85, or a combination thereof; or further comprising detecting an absence of an adenosine (“A”) at a polymorphism position comprising rs1533236, or a proxy polymorphism position in linkage disequilibrium therewith as determined with an r² of at least 0.85, or a combination thereof; or detecting an absence of both, in the plurality of target nucleic acid sequences.

80. A method of enriching a target nucleic acid in a sample, the method comprising:
a) providing a plurality of target nucleic acid sequences obtained from a biological sample from a subject with Crohn's disease (CD);
b) bringing a fluid reaction formulation comprising a plurality of synthetic oligonucleotide molecules in contact with the plurality of target nucleic acid sequences, wherein each synthetic oligonucleotide molecule of the plurality of synthetic oligonucleotide molecules is complementary to at least one target nucleic acid sequence of the plurality of target nucleic acid sequences;
c) hybridizing the plurality of synthetic oligonucleotide molecules to the plurality of synthetic oligonucleotide molecules;
d) amplifying the plurality of target nucleic acid sequences hybridized to the plurality of synthetic oligonucleotide molecules in (c), thereby enriching the plurality of target nucleic acid sequences in the fluid reaction formulation; and
e) detecting the plurality of target nucleic acid sequences that were enriched in (d), wherein the detecting in (e) of the plurality of target nucleic acid sequences is indicative of a high likelihood of post-operative recurrence (“POR”) as compared with a control subject.

81. The method of claim 80, further comprising detecting a presence of a polymorphism comprising an absence of adenosine (“A”) at a polymorphism position comprising rs6811007, or a proxy polymorphism position in linkage disequilibrium therewith as determined with an r² of at least 0.85, or a combination thereof, a presence of a polymorphism comprising an absence of an adenosine (“A”) at a polymorphism position comprising rs1533236, or a proxy polymorphism position in linkage disequilibrium therewith as determined with an r² of at least 0.85, or a combination thereof; or detecting a presence of both, in the plurality of target nucleic acid sequences.

82. The method of any one of claims 78-81, wherein the control subject has CD and has undergone a surgical resection to treat the CD.

83. The method of any one of claims 78-82, wherein the plurality of synthetic oligonucleotide molecules comprises a detectable label.

84. The method of claim 83, wherein the detectable label comprises a fluorescent label, a radioactive label, an enzyme, a chemiluminescent tag, or a colorimetric tag.

85. The method of any one of claims 78-84, wherein detecting the plurality of target nucleic acid sequences comprises performing quantitative polymerase chain reaction (qPCR).

86. The method of any one of claims 78-85, further comprising measuring one or more serological markers in a second sample obtained from the subject.

87. The method of claim 86, wherein the measuring the one or more serological markers comprises introducing a detection agent configured to bind ANCA, ASCA IgA, ASCA IgG to the second sample under conditions sufficient to detect the ANCA, ASCA IgA, ASCA IgG, or combinations thereof, in the second sample.

88. The method of claim 87, wherein the measuring the one or more serological markers in the second sample comprises performing enzyme-linked immunosorbent assay (ELISA).

89. The method of any one of claims 78-88, further comprising analyzing a third sample obtained from the subject for operative visceral adipose tissue (VAT) quality, wherein the operative VAT is defined by a reduced radiodensity.

90. The method of claim 89, wherein the increased radiodensity comprises a VAT radiodensity of at least 0.631 HU.

91. The method of claim 89, wherein the increased radiodensity comprises a VAT radiodensity of ranging from 0.631-0.926 HU.

92. The method of claim 89, wherein the increased radiodensity comprises a VAT:SAT radiodensity of at least 5.559.

93. The method of claim 89, wherein the increased radiodensity comprises a VAT:SAT radiodensity of ranging from 5.56-26.

94. A computing platform for predicting a post-operative prophylaxis persistence (“POPP”) in a subject, the platform comprising:
a) a genotyping device for determining genotype data of a subject with Crohn's disease (CD), wherein the subject is receiving a treatment for the CD comprising a therapeutic agent; and
b) one or more computer processors operably coupled to the genotyping device, wherein the one or more computer processors are individually or collectively configured to perform operations comprising:
i. receiving the genotype data from the genotyping device; and
ii. identifying, in the genotype data, a genotype that is predictive of the POPP.

95. The computing platform of claim 94, further comprising: determining a positive result based on the genotype identified in (ii), wherein the positive result is indicative that the treatment of the CD exhibits a high likelihood to induce POPP in the subject, relative to a control sample obtained from a control subject with CD that has undergone a surgical recission to treat the CD.

96. The computing platform of claim 94 or 95, further comprising detecting an absence of a polymorphism comprising an adenosine (“A”) at rs6811007, or a proxy polymorphism in linkage disequilibrium therewith as determined with an r² of at least 0.85, or a combination thereof; an absence of a polymorphism comprising an adenosine (“A”) at a polymorphism position comprising rs1533236, or a proxy polymorphism position in linkage disequilibrium therewith as determined with an r² of at least 0.85, or a combination thereof; or detecting an absence of both.

97. The computing platform of any one of claims 94-96, wherein a high likelihood of comprises a likelihood of at least about 55%, 60%, 65%, 70%, or 75%.

98. The computing platform of any one of claims 94-96, wherein the operations further comprise receiving POPP marker data for the subject, wherein the POPP marker data comprises POPP clinical factor data, POPP serological factor data, POPP genetic factor data, or any combination thereof.

99. The computing platform of claim 98, wherein the POPP clinical factor data comprises an active smoker status, disease location, a failure of a therapeutic agent to treat the CD, a resection length of operation, or a lymph node granuloma, or any combination thereof.

100. The computing platform of claim 99, wherein the operations further comprise identifying the subject as having a low likelihood of the POPP, wherein the POPP clinical factor data for the subject comprises the active smoker status.

101. The computing platform of claim 100, wherein the active smoker status is defined by smoking at least one cigarette per week.

102. The computing platform of any one of claims 98-101, wherein the operations further comprise identifying a high likelihood that the therapeutic agent exhibits the POPP in treating the CD in the subject, wherein the POPP clinical factor data for the subject comprises the disease location in an ileal region of an intestine of the subject, wherein the high likelihood is relative to a control sample obtained from a control subject with CD that has undergone a surgical recission to treat the CD.

103. The computing platform of any one of claims 98-102, wherein the operations further comprise identifying a high likelihood that the therapeutic agent exhibits the POPP in treating the CD in the subject based, wherein the POPP clinical factor data comprises the disease location in an ileocolonic region of an intestine of the subject, wherein the high likelihood is relative to a control sample obtained from a control subject with CD that has undergone a surgical recission to treat the CD.

104. The computing platform of any one of claims 98-103, wherein the operations further comprise identifying a low likelihood that the therapeutic agent exhibits the POPP in treating the CD in the subject based, wherein the POPP clinical factor data comprises the lymph node granuloma, wherein the low likelihood is relative to a control sample obtained from a control subject with CD that has undergone a surgical recission to treat the CD.

105. The computing platform of any one of claims 98-104, wherein the operations further comprise identifying a low likelihood that the therapeutic agent exhibits the POPP in treating the CD in the subject based, wherein the POPP clinical factors comprises the failure of the therapeutic agent for treatment of the CD in the subject, wherein the low likelihood is relative to a control sample obtained from a control subject with CD that has undergone a surgical recission to treat the CD.

106. The computing platform of claim 105, wherein the therapeutic agent comprises a steroid, an immunomodulator, 5-aminosalicylate, or combinations thereof.

107. The computing platform of claim 105, wherein the therapeutic agent comprises one or more biologic therapeutic agents for treatment of the CD.

108. The computing platform of claim 107, wherein the one or more biologic therapeutic agents for treatment of the CD comprises two or more biologic therapeutic agents.

109. The computing platform of claim 107, wherein the one or more biologic therapeutic agents for treatment of the CD comprises three or more biologic therapeutic agents.

110. The computing platform of any one of claims 105-109, wherein the failure of the therapeutic agent is a recurrence or flare up of the CD following successful remission of the CD following administration of the therapeutic agent to the subject.

111. The computing platform of any one of claims 98-110, wherein the operations further comprise receiving the one or more POPP clinical factors from the subject directly, or receiving medical records of the subject indirectly.

112. The computing platform of any one of claims 98-111, wherein the POPP serological factor data comprises ANCA seronegativity, ASCA IgA seronegativity, or ASCA IgG seronegativity, or any combination thereof.

113. The computing platform of claim 112, wherein the operations further comprise identifying the subject as having seropositivity or seronegativity by calculating a percentage of a reference value from pooled sera obtained from a patient population diagnosed with CD.

114. The computing platform of claim 112 or 113, wherein the operations further comprise identifying a high likelihood that the therapeutic agent exhibits the POPP, wherein the POPP serological factor data comprises ASCA seropositivity, wherein the high likelihood is relative to a control sample obtained from a control subject with CD that has undergone a surgical recission to treat the CD.

115. The computing platform of any one of claims 112-114, wherein the operations further comprise identifying a high likelihood that the therapeutic agent exhibits the POPP, wherein the POPP serological factor data comprises ANCA seronegativity, wherein the high likelihood is relative to a control sample obtained from a control subject with CD that has undergone a surgical recission to treat the CD.

116. The computing platform of any one of claims 112-115, wherein the operations further comprise identifying a high likelihood that the therapeutic agent exhibits the POPP, wherein the POPP serological factor data comprises ASCA seropositivity and ANCA seronegativity, wherein the high likelihood is relative to a control sample obtained from a control subject with CD that has undergone a surgical recission to treat the CD.

117. The computing platform of any one of claims 98-116, wherein the operations further comprise identifying a high likelihood that the therapeutic agent exhibits the POPP, wherein the POPP histological factor data for the subject comprises a operative visceral adipose tissue (VAT) quality, as defined by a reduced radiodensity.

118. The computing platform of claim 117, wherein the reduced radiodensity comprises a VAT radiodensity of less than or equal to about 0.631 Hounsfield unit (HU).

119. The computing platform of claim 117, wherein the reduced radiodensity comprises a VAT radiodensity of about 0.43 to about 0.7 HU.

120. The computing platform of claim 117, wherein the reduced radiodensity comprises a ratio of VAT to SAT radiodensity of up to 5.559.

121. The computing platform of claim 117, wherein the reduced radiodensity comprises a ratio of VAT to SAT radiodensity ranging from 1.18-6.

122. The computing platform of any one of claims 94-121, wherein the genotyping device comprises a nucleic acid sequencer, a nucleic acid array, or a quantitative polymerase chain reaction (qPCR) machine.

123. A computing platform for predicting a post-operative recurrence (“POR”) in a subject, the platform comprising:
a) a genotyping device for determining genotype data of a subject with Crohn's disease (CD), wherein the subject is receiving a treatment for the CD comprising a therapeutic agent; and
b) one or more computer processors operably coupled to the genotyping device, wherein the one or more computer processors are individually or collectively configured to perform operations comprising:
i. receiving the genotype data from the genotyping device; and
ii. identifying, in the genotype data, a predetermined genotype that is predictive of the POR.

124. The computing platform of claim 123, further comprising determining a positive result based on the genotype identified in (ii), wherein the positive result is that the subject exhibits a high likelihood of the POR, relative to a control sample obtained from a control subject with CD that has undergone a surgical recission to treat the CD.

125. The computing platform of claim 123 or 124, wherein the genotype comprises a presence of a polymorphism comprising an adenosine (“A”) at rs6811007, or a proxy polymorphism in linkage disequilibrium therewith as determined with an r² of at least 0.85, or a combination thereof; or wherein the genotype comprises a presence of a polymorphism comprising an adenosine (“A”) at a polymorphism position comprising rs1533236, or a proxy polymorphism position in linkage disequilibrium therewith as determined with an r² of at least 0.85, or a combination thereof; or wherein the genotype comprises detecting an absence of both.

126. The computing platform of any one of claims 124-125, wherein the positive result is further indicative that the subject will not respond to the treatment comprising the therapeutic agent.

127. The computing platform of any one of claims 123-126, wherein operations further comprise receiving POR marker data for the subject, wherein the POR marker data comprises POR clinical factor data, POR serological factor data, POR genetic factor data, or POR histological factor data, or any combination thereof.

128. The computing platform of claim 127, wherein the POR clinical factor data comprises an active smoker status, disease location, a failure of a therapeutic agent to treat the CD, a resection length of operation, or a lymph node granuloma, or any combination thereof.

129. The computing platform of claim 128, wherein the operations further comprise identifying the subject as having a high likelihood of the POR, wherein the one or more POR clinical factors detected in the subject comprise the active smoker status, wherein the high likelihood is relative to a control sample obtained from a control subject with CD that has undergone a surgical recission to treat the CD.

130. The computing platform of claim 128 or 129, wherein the operations further comprise identifying the subject as having a high likelihood of the POR wherein the POR clinical factor data detected comprises the lymph node granuloma, wherein the high likelihood is relative to a control sample obtained from a control subject with CD that has undergone a surgical recission to treat the CD.

131. The computing platform of any one of claims 128-130, wherein the operations further comprise identifying the subject as having a high likelihood of the POR, wherein the POR clinical factor data comprises the disease location in an ileocolonic region of an intestine of the subject, wherein the high likelihood is relative to a control sample obtained from a control subject with CD that has undergone a surgical recission to treat the CD.

132. The computing platform of any one of claims 128-131, wherein the operations further comprise identifying the subject as having a low likelihood of the POR, wherein the POR clinical factor data comprises the disease location in an ileal region of an intestine of the subject, wherein the low likelihood is relative to a control sample obtained from a control subject with CD that has undergone a surgical recission to treat the CD.

133. The computing platform of any one of claims 128-132, wherein the operations further comprise identifying the subject as having a high likelihood of the POR wherein the POR clinical factor data comprises the failure of the therapeutic agent for treatment of the CD, wherein the high likelihood is relative to a control sample obtained from a control subject with CD that has undergone a surgical recission to treat the CD.

134. The computing platform of claim 133, wherein the therapeutic agent comprises an immunomodulator, 5-aminosalicylate, or combinations thereof.

135. The computing platform of claim 133, wherein the therapeutic agent comprises one or more biologic therapeutic agents.

136. The computing platform of claim 135, wherein the one or more biologic therapeutic agents comprises two or more biologic therapeutic agents for the treatment of the CD.

137. The computing platform of claim 135, wherein the one or more biologic therapeutic agent comprises three or more biologic therapeutic agents for the treatment of the CD.

138. The computing platform of any one of claims 127-137, wherein the operations further comprise identifying the subject as having a high likelihood of the POR, wherein the POR serological factor data for the subject comprises ANCA seropositivity, ASCA IgA seropositivity, or ASCA IgG seropositivity, or any combination thereof, wherein the high likelihood is relative to a control sample obtained from a control subject with CD that has undergone a surgical recission to treat the CD.

139. The computing platform of any one of claims 127-138, wherein the operations further comprise identifying the subject as having a high likelihood of the POR, wherein the POR serological factor data comprises ASCA seropositivity, wherein the high likelihood is relative to a control sample obtained from a control subject with CD that has undergone a surgical recission to treat the CD.

140. The computing platform of any one of claims 127-139, wherein the operations further comprise identifying the subject as having a high likelihood of the POR, wherein the POR clinical factor data comprises ANCA seronegativity, wherein the high likelihood is relative to a control sample obtained from a control subject with CD that has undergone a surgical recission to treat the CD.

141. The computing platform of any one of claims 127-140, wherein the operations further comprise identifying the subject as having a high likelihood of the POR, wherein the POR clinical factor data comprises ASCA seropositivity and ANCA seronegativity, wherein the high likelihood is relative to a control sample obtained from a control subject with CD that has undergone a surgical recission to treat the CD.

142. The computing platform of any one of claims 127-141, wherein the POR histological factor data comprises an operative visceral adipose tissue (VAT) quality.

143. The computing platform of claim 142, wherein the operations further comprise identifying the subject as having a high likelihood of the POR, wherein the operative VAT quality is defined by an increased radiodensity.

144. The computing platform of claim 143, wherein the increased radiodensity comprises a VAT radiodensity of at least 0.631 HU.

145. The computing platform of claim 143, wherein the increased radiodensity comprises a VAT radiodensity of ranging from 0.631-0.926 HU.

146. The computing platform of claim 143, wherein the increased radiodensity comprises a VAT:SAT radiodensity of at least 5.559.

147. The computing platform of claim 143, wherein the reduced radiodensity comprises a VAT:SAT radiodensity of ranging from 5.56-26.

148. A kit for predicting a post-operative prophylaxis persistence (“POPP”) in a subject having CD, the kit comprising:
a) one or more detection agents configured to bind or hybridize to a polymorphism comprising an adenosine (“A”) at rs6811007 or a proxy polymorphism in linkage disequilibrium (LD) therewith as determined with an r² of at least 0.85, or a combination thereof, or to bind or hybridize to a polymorphism comprising an adenosine (“A”) at a polymorphism position comprising rs1533236, or a proxy polymorphism position in linkage disequilibrium therewith as determined with an r² of at least 0.85, or a combination thereof, or multiple detection agents configured to bind or hybridize to both; and
b) instructions for use of the detection agent to detect an absence of the polymorphism or the proxy polymorphism in LD therewith in a sample obtained from the subject.

149. The kit of claim 148, further comprising:
a) a second detection agent configured to bind ANCA, ASCA IgA, ASCA IgG, or combinations thereof; and
b) instructions for use of the second detection agent to detect seronegative or seropositivity of the ANCA, the ASCA IgA, the ASCA IgG, or any combinations thereof in a sample from the subject.

150. The kit of claim 148 or 149, wherein the instructions further comprise instructions for predicting the POPP in the subject.

151. A kit for predicting a post-operative recurrence (“POR”) in a subject having CD, the kit comprising:
a) one or more detection agent(s) configured to bind or hybridize to a polymorphism comprising an adenosine (“A”) at rs6811007, or a proxy polymorphism in linkage disequilibrium (LD) therewith as determined with an r² of at least 0.85, or a combination thereof, configured to bind or hybridize to a polymorphism comprising an adenosine (“A”) at a polymorphism position comprising rs1533236, or a proxy polymorphism position in linkage disequilibrium therewith as determined with an r² of at least 0.85, or a combination thereof, or multiple detection agents configured to bind or hybridize to both; and
b) instructions for use of the detection agent to detect a presence of the polymorphism or the proxy polymorphism in LD therewith in a sample obtained from the subject.

152. The kit of claim 151, further comprising:
a) a second detection agent configured to bind ANCA, ASCA IgA, ASCA IgG in a sample from the subject; and
b) instructions for use of the second detection agent to detect seronegative or seropositivity of the ANCA, the ASCA IgA, the ASCA IgG, or any combinations thereof in a sample from the subject.

153. The kit of claim 151 or 152, wherein the instructions further comprise instructions for predicting the POR in the subject.
